(19) United States
(12) Patent Application Publication
(10) Pub. No.: US 2020/0289554 A1
(43) Pub. Date: Sep. 17, 2020

(54) BIOORTHOGONAL COMPOUNDS COMPRISING A PROPARGYL GROUP FOR TREATING CANCER

(71) Applicant: THE UNIVERSITY COURT OF THE UNIVERSITY OF EDINBURGH, Midlothian (GB)

(72) Inventors: Belen RUBIO-RUIZ, Midlothian (GB); Thomas L BRAY, Midlothian (GB); Ana M PEREZ-LOPEZ, Midlothian (GB); Catherine ADAM, Midlothian (GB); Asier UNCITI-BROCETA, Midlothian (GB)

(21) Appl. No.: 16/302,671

(22) PCT Filed: May 17, 2017
(86) PCT No.: PCT/GB2017/051379

§ 371 Date: Nov. 18, 2018

(30) Foreign Application Priority Data
May 18, 2016 (GB) 1608716.5

(51) Int. Cl. A61K 33/242; A61K 31/351; A61K 31/506; A61K 31/502; A61K 31/4174; A61K 31/437; A61K 31/36

(52) U.S. Cl. CPC A61K 33/242; A61K 31/351; A61K 31/506; A61K 31/18; A61K 31/4174; A61K 31/437; A61K 31/36; A61K 31/502

(57) ABSTRACT

A method of preparing an active agent or a salt thereof from a pro-drug first compound (1) comprising a propargyl group connected to an oxygen that is directly or indirectly connected to the active agent is provided, wherein the bond between the propargyl group and the oxygen is cleaved by reacting the first compound with palladium or gold, thereby releasing the active agent. Prodrug compositions suitable for use in the method are also provided.

### FIELD OF THE INVENTION

The present invention relates to bioorthogonal deprotection methods, and to compounds for use in such methods, including prodrug forms of active agents that can be converted to the active agent in situ by palladium or gold catalysis.

### BACKGROUND OF THE INVENTION

Bioorthogonal Chemistry

As reported by Bertozzi, et al. in the early 2000's (Bertozzi, C. R. et al. Science, 2000, 287, 2007-2010 and Bertozzi, C.R. et al. J. Am. Chem. Soc. 2004, 126, 15046-15047), artificial synthetic chemistry can be conducted in a biological environment without adverse biological effects using highly chemospecific reactive partners. Such reactions which proceed in a biological environment without adverse biological consequences are now commonly referred to as being “bioorthogonal”.

Initial bioorthogonal studies focussed on the development of labelling strategies based on the selective conjugation of two biologically-inert functional groups. This development has since enabled the real-time study of a wide range of biomolecules in their native environs (see, e.g. Bertozzi, C. R. Ace Chem Res. 2011, 44, 651-653).

Transition Metal Catalysed Reactions

Transition metal catalysed reactions are an extremely powerful tool in organic synthesis as they provide chemospecific reaction profiles and facilitate a wide range of chemical transformations. From a bioorthogonal synthetic perspective, it is therefore desirable to develop bioorthogonal transition metal catalysed reactions that can perform efficiently in a biological environment to provide the biosynthetic chemist with more synthetic flexibility.

A large variety of transition metal catalysed reactions have been reported in the literature. However, there has been limited success in the application of such reactions in a biological environment. This is potentially due to a large number of reported reaction conditions being simply incompatible with a biological environment, e.g. requiring organic solvents and/or high temperatures, etc. For instance, the palladium-mediated cleavage of propargyl protecting groups from aryl amines requires biologically incompatible temperatures of at least 80° C. (see, e.g. Pal, M. et ot., Org. Lett. 2003, 5(3), 349-352).

Certain non-biological transition metal-catalysed reactions have however been shown to be promising candidates for use in bioorthogonal synthesis (e.g. Unciti-Broceta, A. et al. Nature Protocols, 2012, 7, 1207-1218 and Meggers, E. et al. Chem Commun. 2013, 49, 1581-1587). Such bioorthogonal organometallic (BOOM) reactions are biocompatible and involve chemospecific transformations undertaken usually by synthetic materials and mediated by a non-biotic metal source as described below.

In 2006, Meggers et al. described the application of a water-soluble ruthenium-based catalyst to carry out Allyl carbamate (Alloc) deprotection of bis-N,N′-allyloxycarbonyl rhodamine 110 inside human cells without adversely affecting cell viability (Meggers, E. et al., Angew. Chem. Int. Ed, 2006, 45, 5645-5648). The use of palladium-functionalized microspheres as a heterogeneous catalyst medium for promoting BOOM chemistry inside cells has also been reported (Bradley, M. et al., Nat. Chem. 2011, 3, 239-243 and Unciti-Broceta, A. et al. Nature Protocols, 2012, 7, 1207-1218). The palladium-functionalized microspheres were shown to be able to enter cells in vitro and catalyse Alloc deprotection and Suzuki-Miyaura cross-coupling in the cell cytoplasm without any observed cytotoxicity.

Biomedical Applications

In biomedicine, bioorthogonal deprotection methods could be utilised to transform a bioorthogonal chemical into a bioactive material. Prodrugs, for example, are active agent precursors that are converted to the active agent following administration to a patient, typically by chemical rearrangement of the prodrug and/or by cleavage of a pro-moiety by natural biological metabolism. Typically, prodrugs are based on active agents that have been protected with a cleavable protecting group or pro-moiety. By providing an active agent precursor that produces the active agent within the body, compounds can be produced that exhibit improved pharmacokinetic properties compared to the active drug, such as greater oral bioavailability and sustained release profiles.

For safety and simplicity, it is desirable to provide prodrugs that do not exhibit biological activity themselves. The activity profile of the prodrug is then entirely dependent on the metabolic conversion of the prodrug to the active agent, providing a greater degree of predictability of biological activity in vivo.

Typically, prodrugs are converted to the respective active agents in the gut (for orally administered drugs), and/or by general cellular and/or plasma-based metabolic pathways. Conventional prodrugs are thus converted to the active agent in a non-bioselective manner, leading to general systemic exposure of the body cells to the active agent, which may result in undesirable side effects.

It is therefore desirable from a toxicological perspective to be able to deliver active agents specifically to the relevant target/disease site, and to prevent the active agent acting on the rest of the body cells.

Accordingly, it is one object of the present invention to provide a method of delivering an active agent to a target site without substantially exposing the body cells outside of the target site to the active agent.

### SUMMARY OF THE INVENTION

According to a first aspect of the invention there is provided a method of preparing an active agent or a salt thereof, the method comprising the steps:
a) providing a first compound defined according to formula (1):

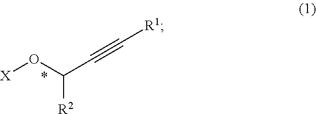

and
b) cleaving the bond (*) between the oxygen and the propargyl group by reacting the first compound with palladium or gold;
wherein R¹ and R² are independently selected from the group consisting of H, optionally substituted C₁-C₁₀ alkyl, optionally substituted C₃-C₁₀ cycloalkyl, optionally substituted C₂-C₁₀ alkenyl, optionally substituted C₃-C₁₀ cycloalkenyl, optionally substituted C₂-C₁₀ alkynyl, optionally substituted C₂-C₁₀ heteroalkyl, optionally substituted C₃-C₁₀ heterocycloalkyl, optionally substituted C₂-C₁₀ heteroalkenyl, optionally substituted C₃-C₁₀ heterocycloalkenyl, optionally substituted C₂-C₁₀ heteroalkynyl, optionally substituted C₆-C₁₄ aryl, optionally substituted C₅-C₁₄ heteroaryl,
wherein X—O comprises at least one aryl group or heteroaryl group directly connected to the oxygen (O) of the X—O substituent, and comprises the active agent or a salt thereof, and optionally comprises a linker between the oxygen and the active agent.

For the avoidance of doubt, the bond (*) between the oxygen and the propargyl group may be cleaved by reacting the first compound with palladium. The bond (*) between the oxygen and the propargyl group may be cleaved by reacting the first compound with gold. In some embodiments the bond (*) between the oxygen and the propargyl group may be cleaved by either palladium or gold. Accordingly, the method of the present aspect may be carried out by contacting a composition comprising the first compound with source of palladium, a source of gold, or a source of both palladium and gold.

The X—O group may comprise a derivative of the active agent.

Typically, the bond (*) is a covalent bond and this bond is not readily cleaved under natural metabolic conditions. However, the bond (*) has been found to be cleavable by palladium or gold under ambient conditions. For example, the bond (*) may be cleaved under biologically compatible conditions (e.g. in aqueous solution, such as buffered solution at physiological pH and at around 37° C.). Accordingly, the bond cleavage may be performed in aqueous media. The reaction may be performed at around physiological pH, i.e. the reaction may be performed from about pH 6-8, preferably, from about pH 6.5-7.5. The reaction may be performed at around 37° C. In some embodiments the method of the invention is performed at a temperature of 100° C. or less, such as 90° C. or less, for example, 80, 70, 60, 50, or 40° C. or less. For biological applications, the reaction temperature is preferably 40° C. or less, typically less than 40° C., preferably around 37° C.

Typically, the bond cleavage in the present methods proceeds efficiently in biocompatible conditions to provide the desired active agent or the linker and active agent. In embodiments where the bond cleavage proceeds to provide a linker connected to an active agent, the exposed linker typically rapidly breaks down in biocompatible conditions to leave the free active agent. In embodiments, the methods of the invention proceed to at least 10% completion (i.e. wherein at least 10% of the starting compound has been cleaved to provide the active agent) within 72 h from when reaction with the palladium commences. Suitably, the methods of the invention proceed to at least 20% completion within 72 h, such as at least 30%, 40%, 50%, 60%, 70%, 80%, 90%, 95%, 98%, preferably 99% and more preferably 100% completion within 72 h, more preferably within 48 h, such as within 24 h, e.g. within 10 h.

It is believed that the mechanism of cleavage of such propargyl groups by palladium as described in embodiments of the present methods principally involves the steps of coordination of palladium to the triple bond of formula (1), followed by insertion of the palladium into the triple bond (oxidatively or ionically). Oxidative addition for instance is typically observed when palladium(0) is used as the palladium source, resulting in formation of an allenyl palladium intermediate. The X—O group is eliminated, thus breaking the bond (*) between the X—O group and the propargyl group.

Suitably, the methods of the present invention may therefore be performed in a biological environment, such as in a cell, a tissue and/or a subject using a suitable palladium source and/or a suitable gold source. As a result, the reaction may suitably be performed in vivo by administration of the compound according to formula (1) and palladium or gold to a subject. Modes of administration are discussed further below.

Alternatively, the reaction may be performed in vitro.

Thus, prodrugs that may be converted to active agent in a spatially controlled manner may offer a way to enable active agent to be produced only where it is needed, i.e. at specific target sites, such as a specific disease site in the body, thus minimising the general systemic exposure of the patient to the active agent. Importantly, a spatially-targeted approach would serve to expand the therapeutic window and scope of potent cytotoxic drugs such as 5-FU, which have a long history in oncology practice but a clinical activity limited by its safety profile (Chu, et al. J. Natl. Cancer Inst. 101, 1543 (2009)), and to allow the medical application of highly promising experimental drugs that failed to progress through clinical trials to approval due to toxicological issues. An appropriate prodrug strategy may therefore allow a wide range of active agents to reach the clinic in an optimized manner.

Ideally, the bioorthogonality of the prodrug should be two-fold: the prodrug should preferably neither interact with the therapeutic target/s nor be biochemically metabolized into the active agent (unlike conventional prodrugs). This behaviour may be attained by modifying an active agent structure at a position that is mechanistically-relevant to its pharmacological activity with chemical groups that cannot be easily recognized by human enzymes. The design of a suitable masking strategy is therefore an important aspect of this approach. On the other hand, the corresponding metallic agent needs to be biocompatible (ideally bioorthogonal) and able to coordinate with and cleave the active agents masking group in physiological conditions (aqueous solvent, physiological temperature, pH, etc.). Importantly, the active oxidation state of the metal therefore needs to be compatible with the inherent redox potential of the biological environment. Preferably, the bioorthogonal organometallic (BOOM) reaction should also be catalytic, to allow a repetitive dosing regimen to be implemented.

The active agent may be a therapeutic active agent. The active agent may be a cytotoxic active agent that may be used to treat cancer, for example. Accordingly, in this embodiment the active agent may only be released from the propargyl group and, where present, a linker group when the compound of formula (1) comes into contact with a palladium-containing implant in the tumour, for example. In a further example, the active agent may only be released from the propargyl group and, where present, a linker group when the compound of formula (1) comes into contact with a gold-containing implant in the tumour.

Typically, R¹ and R² are independently selected from the group consisting of H, optionally substituted C₁-C₅ alkyl, optionally substituted C₃-C₆ cycloalkyl, optionally substituted C₂-C₆ alkenyl, optionally substituted C₃-C₆ cycloalkenyl, optionally substituted C₂-C₅ alkynyl, optionally substituted C₂-C₅ heteroalkyl, optionally substituted C₃-C₆ heterocycloalkyl, optionally substituted C₂-C₅ heteroalkenyl, optionally substituted C₃-C₆ heterocycloalkenyl, optionally substituted C₂-C₅ heteroalkynyl, optionally substituted C₆-C₁₂ aryl, optionally substituted C₅-C₁₁ heteroaryl.

Preferably, R¹ and R² are independently selected from the group consisting of H, optionally substituted C₁-C₄ alkyl, optionally substituted C₂-C₄ alkenyl, optionally substituted C₂-C₅ alkynyl, optionally substituted C₂-C₅ heteroalkyl, optionally substituted C₂-C₅ heteroalkenyl, optionally substituted C₂-C₅ heteroalkynyl.

For example, the first compound may have a general formula selected from the group comprising:

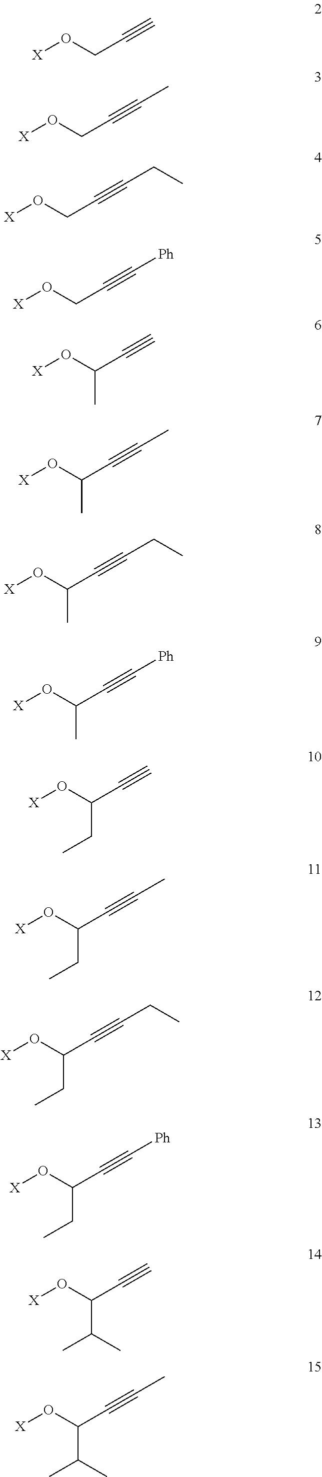

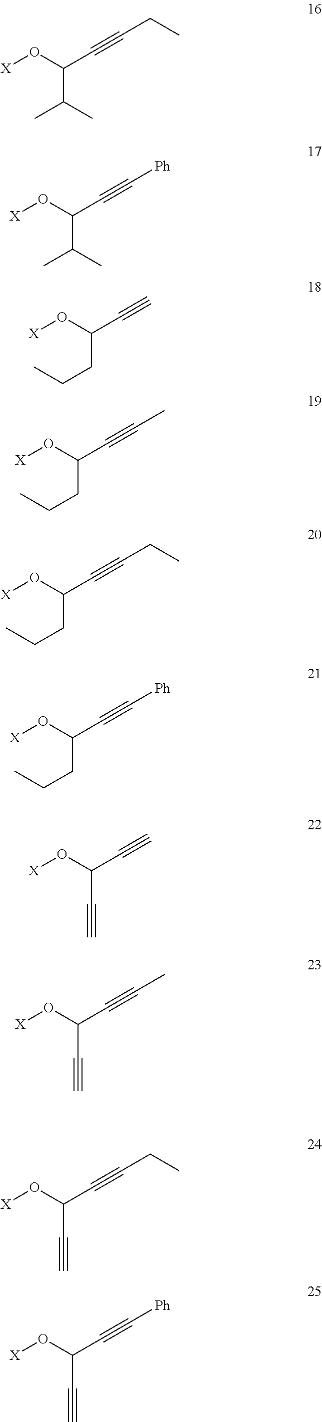

In embodiments where the X—O group comprises an active agent and a linker, the active agent may be connected to the linker via an amine, hydroxyl, hydroxamic acid or carbonyl group of the active agent. The linker may comprise an aryl or heteroaryl group connecting the oxygen of the X—O group to the active agent. The linker may comprise an alkyl aryl or alkyl heteroaryl group connecting the oxygen of the X—O group to the active agent. For example, the linker may comprise an alkyl substituted benzene group, such that the linker and the oxygen of the X—O group form an alkyl phenyl group. Typically, the alkyl group is para- or ortho- to the oxygen on the aromatic ring. The linker may further comprise a carboxylic acid ester group that may be released as CO₂ when the bond (*) is cleaved by palladium or gold.

In preferred embodiments, the linker is selected from the group

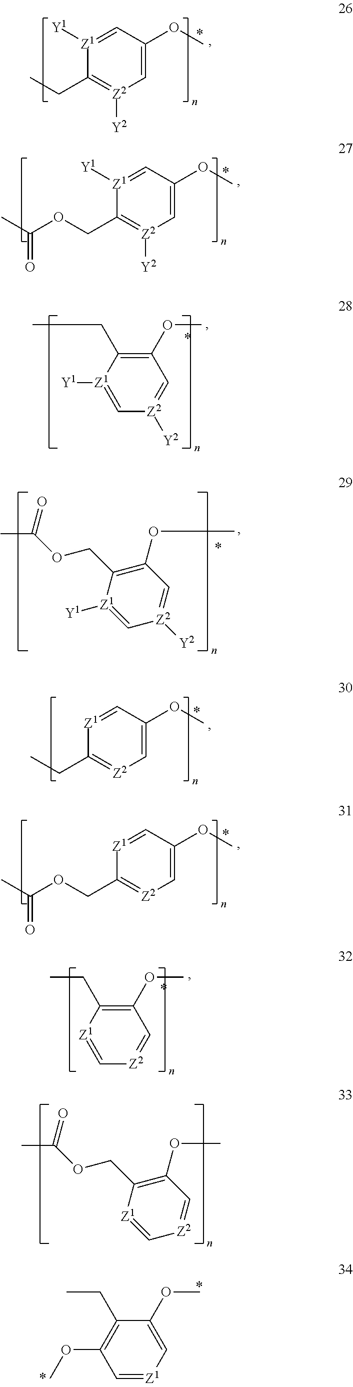

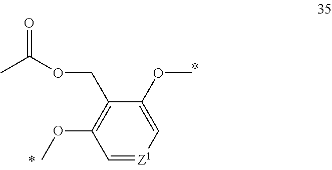

wherein
Z¹ and Z² are independently selected from N, CH, C;
Y¹ and Y² are independently selected from H, NO₂, halogen, COOR³, OR⁴;
R³ and R⁴ are independently selected from the group consisting of H, optionally substituted C₁-C₁₀ alkyl, optionally substituted C₃-C₁₀ cycloalkyl, optionally substituted C₂-C₁₀ alkenyl, optionally substituted C₃-C₁₀ cycloalkenyl, optionally substituted C₂-C₁₀ alkynyl, optionally substituted C₂-C₁₀ heteroalkyl, optionally substituted C₃-C₁₀ heterocycloalkyl, optionally substituted C₂-C₁₀ heteroalkenyl, optionally substituted C₃-C₁₀ heterocycloalkenyl, optionally substituted C₂-C₁₀ heteroalkynyl, optionally substituted C₆-C₁₄ aryl, optionally substituted C₅-C₁₄ heteroaryl; and
n is 1-10, preferably 1, 2 or 3.

It is important for effective delivery of the active agent that the activation rate of the active agent in a target area (i.e. the conversion of the prodrug of formula (1) to the active agent) is significantly greater than the rate that the biological system within which the target area is located clears the prodrug or active agent from the target area. Accordingly, it is advantageous for the cleavage of the bond (*) between the oxygen and propargyl group in formula (1) to be fast in the presence of palladium or gold, and slow in the absence of palladium or gold.

Thus, compounds according to formula (1) used in the present methods are useful prodrugs that may be suitably deprotected in a controlled manner using palladium or gold to reveal the active agent, e.g. in vitro or in vivo. In typical embodiments, the active agent may be an anti-cancer active agent. The active agent may be suitable to treat any solid tumour cancer. In preferred embodiments, the anti-cancer active agent may be selected from active agents for treating pancreatic and/or colorectal cancer, prostate cancer, ovarian cancer, breast cancer, lung cancer, liver cancer or brain cancer, for example.

The active agents may contain a hydroxamic acid group connected to the propargyl group directly or via a linker. For example, the active agent may be vorinostat, belinostat, panobinostat, and derivatives thereof.

In embodiments where the first compound comprises a hydroxamic acid group connected to the propargyl group directly or via a linker, it has been surprisingly found that direct alkylation of the OH of the active agent's hydroxamic acid group leads to a significant reduction of bioactivity with a projected therapeutic index far beyond two orders of magnitude.

It has previously been shown by Cohen et al. (Chem. Commun. 2011, 47, 7968-7970) that 4-hydroxybenzyl benzohydroxamate (an aromatic hydroxamic acid) is stable in water at pH=7.5, suggesting that 1,6-elimination of the 4-hydroxybenzyl moiety would not take place in biological environs. However, the inventors have surprisingly shown that in embodiments where the X—O group comprises an active agent comprising a hydroxamic acid connected to a linker comprising a 1,6-methyl phenol moiety, the deprotection mechanism is a tandem reaction triggered by palladium or gold catalysis via depropargylation of the phenolic OH group and followed by 1,6-elimination of a 4-hydroxybenzyl group directly attached to the OH of the active agent's hydroxamic acid group, and that the reaction takes place in biocompatible conditions.

The active agent may contain primary or secondary amino groups connected to the oxypropargyl group directly or via a linker. For example, the active agent may be doxorubicin, gemcitabine, histamine, mitoxantrone, panobinostat, hydroxyurea, paclitaxel, phosphoramide mustard, procarbazine, 5-(monomethyl triazine)-imidazole-4-carboxamide, dasatinib, erlotinib, bosutinib, gefitinib, lapatinib, vandetanib, pazopanib, crizotinib, ceritinib, afatinib, ibrutinib, dabrafenib, trametinib, palbociclib, spanisertib and derivatives thereof.

The active agent may comprise a phenolic OH connected to the oxypropargyl group directly or via a linker, including the equivalent lactam tautomers. For example, the active agent may be 5-fluorouracil (5-FU or 5FU), floxuridine, olaparib, permetrexed, sunitinib, nintedanib, doxorubicin, mitoxantrone, 4-hydroxytamoxifen, SN-38 (active metabolite of irinotecan), etoposide, duocarmycin and derivatives thereof.

The inventors have surprisingly found that the depropargylation of Ar—O-propargyl is significantly faster than R—O-propargyl, where R is non-aryl, and that this form of the prodrug is much more biochemically stable (i.e. is bioorthogonal), and thus induce a much larger difference in bioactivity between the prodrug form and the active therapeutic agent. This results in an increment of the therapeutic window, which could enable further increasing prodrug doses administered to patients while reducing side effects.

In some embodiments, the first compound may comprise a plurality of propargyl groups connected to an oxygen which is in turn connected to an aryl group. The aryl group may be part of a linker. The aryl group may be part of the active agent. For example, the first compound may comprise two, three or four propargyl-oxygen groups. An example of an embodiment comprising two propargyl-oxygen groups is shown as Formula (36):

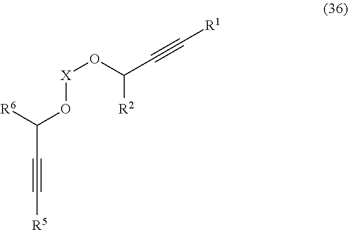

wherein R¹, R², R⁵ and R⁶ are independently selected from the group consisting of H, optionally substituted C₁-C₁₀ alkyl, optionally substituted C₃-C₁₀ cycloalkyl, optionally substituted C₂-C₁₀ alkenyl, optionally substituted C₃-C₁₀ cycloalkenyl, optionally substituted C₂-C₁₀ alkynyl, optionally substituted C₂-C₁₀ heteroalkyl, optionally substituted C₃-C₁₀ heterocycloalkyl, optionally substituted C₂-C₁₀ heteroalkenyl, optionally substituted C₃-C₁₀ heterocycloalkenyl, optionally substituted C₂-C₁₀ heteroalkynyl, optionally substituted C₆-C₁₄ aryl, optionally substituted C₅-C₁₄ heteroaryl,
and wherein O—X—O comprises at least one aryl group or heteroaryl group directly connected to each oxygen (O) of the O—X—O substituent, and comprises the active agent or a salt thereof, and optionally one or two linkers.

The same aryl group of the active agent may be directly connected to each oxygen-propargyl group. Alternatively, different aryl groups of the active agent may be directly connected to each oxygen-propargyl group.

Accordingly, the compound according to formula (1) may be selected from the following group:

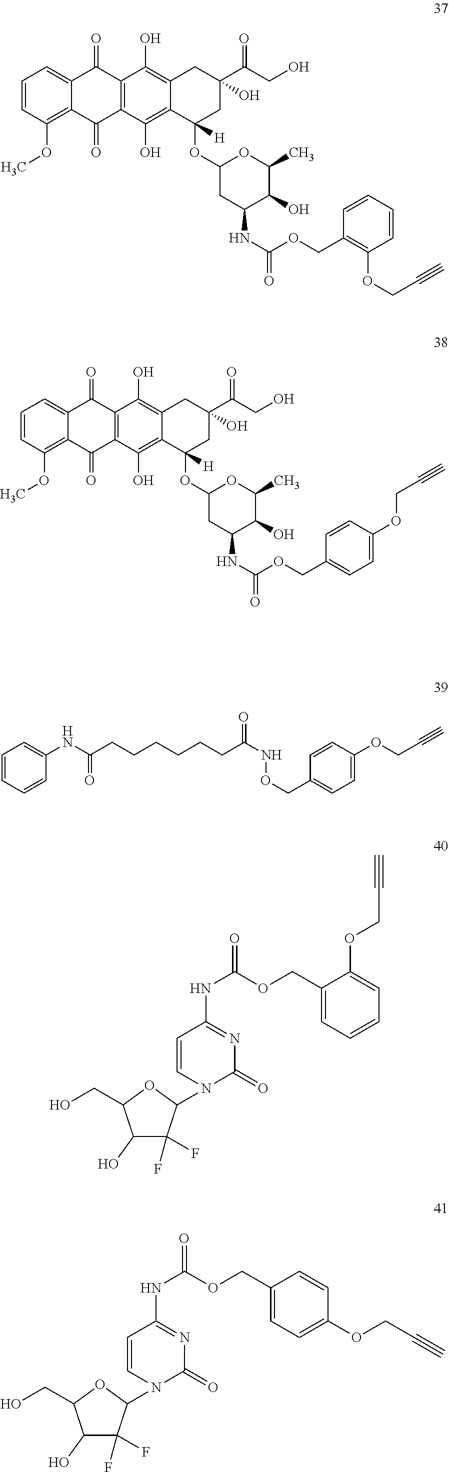 [SMILES: C#CCOc1ccccc1COC(=O)N[C@H]1CC(O[C@H]2C[C@](O)(C(=O)CO)Cc3c(O)c4c(c(O)c32)C(=O)c2c(OC)cccc2C4=O)O[C@@H](C)[C@H]1O ; C#CCOc1ccc(COC(=O)N[C@H]2CC(O[C@H]3C[C@](O)(C(=O)CO)Cc4c(O)c5c(c(O)c43)C(=O)c3c(OC)cccc3C5=O)O[C@@H](C)[C@H]2O)cc1 ; C#CCOc1ccc(CONC(=O)CCCCCCC(=O)Nc2ccccc2)cc1 ; C#CCOc1ccccc1COC(=O)Nc1ccn(C2OC(CO)C(O)C2(F)F)c(=O)n1 ; C#CCOc1ccc(COC(=O)Nc2ccn(C3OC(CO)C(O)C3(F)F)c(=O)n2)cc1]

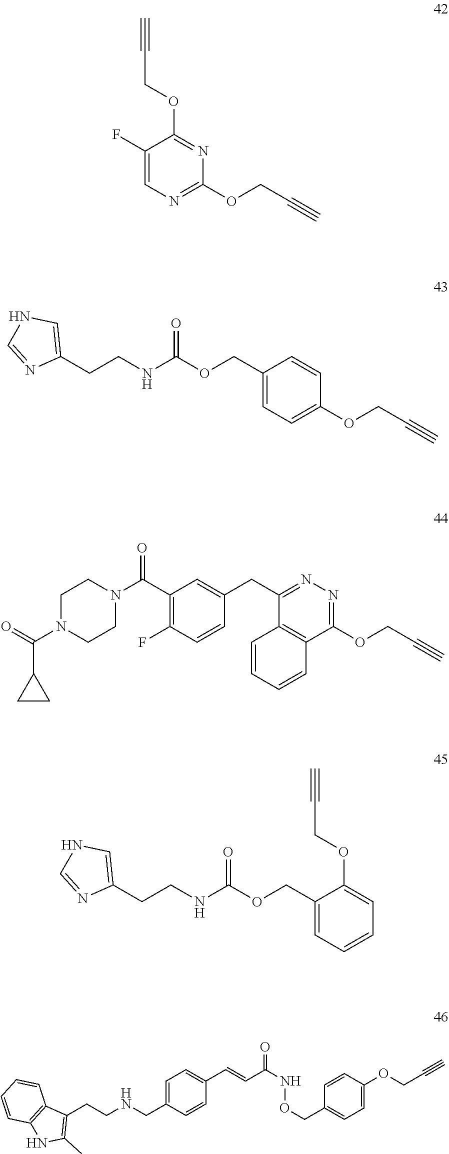 [SMILES: C#CCOc1ncc(F)c(OCC#C)n1 ; C#CCOc1ccc(COC(=O)NCCc2c[nH]cn2)cc1 ; C#CCOc1nnc(Cc2ccc(F)c(C(=O)N3CCN(C(=O)C4CC4)CC3)c2)c2ccccc12 ; C#CCOc1ccccc1COC(=O)NCCc1c[nH]cn1 ; C#CCOc1ccc(CONC(=O)/C=C/c2ccc(CNCCc3c(C)[nH]c4ccccc34)cc2)cc1]

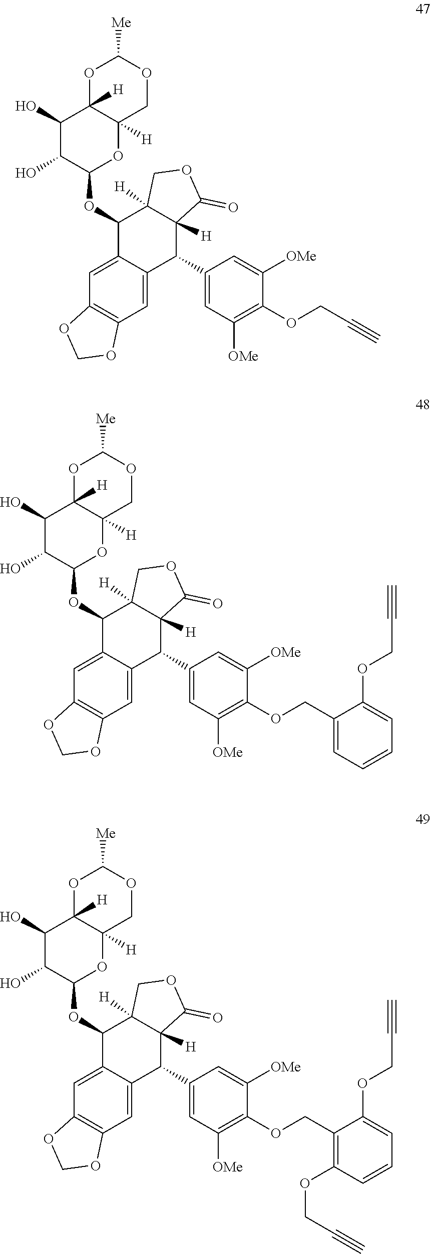

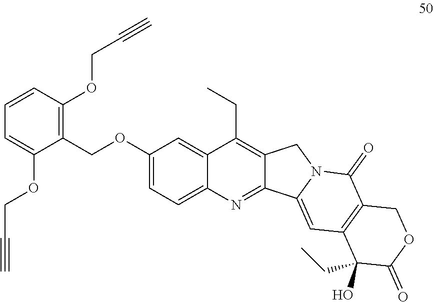 [SMILES: C#CCOc1cccc(OCC#C)c1COc1ccc2nc3c(c(CC)c2c1)Cn1c-3cc2c(c1=O)COC(=O)[C@]2(O)CC]

As such, for biological applications, e.g. in in vivo application the compound may be administered to a subject in which palladium is present and in a manner that allows contact between the compound and palladium so that the active agent or salt thereof as described above is generated in the body. The compound may be administered to a subject in which gold is present and in a manner that allows contact between the compound and gold so that the active agent or salt thereof as described above is generated in the body. The compound may be administered to a subject in which both palladium and gold is present and in a manner that allows contact between the compound and palladium or gold so that the active agent or salt thereof as described above is generated in the body. Methods of administration of compounds of the invention and palladium and/or gold are discussed further below.

For instance, palladium and/or gold may be provided by any convenient means, e.g. as a fluid solution containing the palladium and/or gold, or as a colloidal solution containing palladium nanoparticles and/or gold nanoparticles. Suitable ligand systems for use in forming a fluid solution or for chelating the palladium and/or gold to a solid phase medium such as a particle/implant will be apparent to the skilled person.

In embodiments, the palladium and/or gold is conjugated to another molecule. Suitably the palladium and/or gold may be conjugated to a peptide, polynucleic acid (polynucleotide), or fluorogenic tag, preferably a peptide or polynucleic acid. For instance, the palladium may be conjugated to an antibody or aptamer. The gold may be conjugated to an antibody or aptamer. For example, by conjugating the palladium or gold to an antibody or aptamer, the palladium or gold may be delivered to a specific target site in the body (by virtue of the specific interaction between target antigen and the antibody or aptamer and target site in the body) ready for performing the bond cleavage reaction according to the method of the present invention.

In preferred embodiments, the palladium or gold is provided in the form of an implant, which may be located at a therapeutically important location in the body, e.g at, in, adjacent or near a tissue requiring treatment with the therapeutically active form of the drug, such as at, in, adjacent or near a tumour. Advantageously, if the palladium or gold is provided as an extracellular implant, once the relevant condition has been treated (e.g. once a cancer tumour has shrunk to a safe healthy-to-tumoural tissue ratio), the palladium or gold may be safely removed by surgery (e.g. along with any residual tumour in the case of cancer treatment).

Palladium or gold bonded in solid phase may take a number of physical forms. For instance, the palladium may be provided as a palladium implant (i.e. for administration to a patient), or the gold may be provided as a gold implant. Such implants may have a range of physical forms, the intention being that the implant retains the palladium or gold substantially at or near the site of administration/implantation thereby providing a localised concentration of palladium and/or gold and preventing unwanted high levels of palladium or gold circulating throughout the body. Examples of a palladium implant include a material coated or impregnated by palladium or by a palladium containing compound, such as a palladium-containing alloy. Examples of a gold implant include a material coated or impregnated by gold or by a gold containing compound, such as a gold-containing alloy. The material may be a solid (e.g. a porous solid) or semi-solid, e.g. a gel, and may be in the form of a bolus. The implant should allow for contact of prodrug present in the tissue or associated vasculature with the palladium or gold present in the implant.

The implant material may be selected to allow the coated or impregnated palladium or gold to be released from the material when administered to or implanted in the subject. Release kinetics may be altered by altering the structure, e.g. porosity, of the material.

Typically, the material provides a scaffold or matrix support for the palladium or gold. The material may be suitable for implantation in tissue, or may be suitable for administration to the body (e.g. as microcapsules in solution).

Preferably, the implant material should be biocompatible, e.g. non-toxic and of low immunogenicity (most preferably non-immunogenic). The biomaterial may be biodegradable such that the biomaterial degrades over time. Alternatively a non-biodegradable biomaterial may be used, allowing surgical removal of the implant as required.

Suitable materials may be soft and/or flexible, e.g, hydrogels, fibrin web or mesh, wafers or collagen sponges. A “hydrogel” is a substance formed when an organic polymer, which can be natural or synthetic, is set or solidified to create a three-dimensional open-lattice structure that entraps molecules of water or other solutions to form a gel. Solidification can occur by aggregation. coagulation, hydrophobic interactions or cross-linking.

Alternatively suitable materials may be relatively rigid structures, e.g. formed from solid materials such as plastics, resins or biologically inert metals such as titanium. The implant material may have a porous matrix structure which may be provided by a cross-linked polymer. Matrix structures may be formed by crosslinking fibres, e.g. fibrin or collagen, or of liquid films of sodium alginate, chitosan, or other polysaccharides with suitable crosslinkers, e.g. calcium salts, polyacrylic acid, heparin. Alternatively scaffolds may be formed as a gel, fabricated by collagen or alginates, crosslinked using well established methods known to those skilled in the art.

Suitable polymer materials for matrix formation include, but are not limited by, biodegradable/bioresorbable polymers which may be chosen from the group of: agarose, collagen, fibrin, chitosan, polycaprolactone, poly(DL-lactide-co-caprolactone), poly(L-lactide-co-caprolactone-co-glycolide), polyglycolide, polylactide, polyhydroxyalcanoates, co-polymers thereof; or non-biodegradable polymers which may be chosen from the group of: polystyrene, polyethylene glycol, cellulose acetate; cellulose butyrate, alginate, polysulfone, polyurethane, polyacrylonitrile, sulfonated polysulfone, polyimide, polyacrylonitrile, polymethylmethacrylate, co-polymers thereof. Preferably the non-biodegradable polymer is polystyrene, polyethylene glycol, or a polystyrene-polyethylene glycol copolymer.

Other suitable materials include ceramic or metal (e.g. titanium), hydroxyapatite, tricalcium phosphate, demineralised bone matrix (DBM), autografts (i.e. grafts derived from the patient's tissue), or allografts (grafts derived from the tissue of an animal that is not the patient). Implant materials may be synthetic (e.g metal, fibrin, ceramic) or biological (e.g. carrier materials made from animal tissue, e.g. non-human mammals (e.g. cow, pig), or human).

One form of commercially available palladium implant is a palladium seed implant such as the TheraSeed™ (Theragenics Corporation, Buford, Ga., USA), which is used as a brachytherapy biocompatible device but could be adapted to the purpose of this invention (using it in a nonradioactive form).

In a preferred embodiment of a palladium implant, the polymer material is polyethylene glycol (PEG)-polystyrene graft co-polymer in which the PEG chains have been terminally functionalized with an amino group (e g. NovaSyn® TG amino resin). This polymer has been previously functionalized with Pd⁰ nanoparticles by: (i) mixing with Pd(OAc)₂, (ii) in situ reduction to Pd⁰ and (iii) intensive cross-linking of the polymer surface with activated diacyl compounds to physically trap the Pd⁰ nanoparticles in the polymer (Cho, et al. J. Am. Chem. Soc. 128, 6276-6277 (2006)). The Pd⁰ functionalized polymer demonstrated high catalytic activity in water and remarkable reusability properties (over 10 catalytic cycles without reducing performance).

In some embodiments, the palladium may include palladium nanoparticles, such as described in Nature Protocols, 7, 1207-1218 (2012), Pd⁰-functionalized polystyrene microspheres, such as described in Yusop et al., Nat. Chem. 2011, 3, 239-243,. Pd⁰-functionalized polyethylene glycol polyacrylamide copolymer (PEGA) resins, and PEG-polystyrene graft co-polymer in which the PEG chains have been terminally functionalized with an amino group (e.g. NovaSyn® TG amino resin, which is a 3000-4000 M.W.) such as described in Cho et al. J. Am. Chem. Soc. 128, 6276-6277 (2006). Preferably, the palladium is provided as a palladium functionalized PEG-polystyrene composite resin.

In some embodiments, the gold may include gold nanoparticles or ions, gold-functionalized polystyrene resins such as described by Cao et al Adv. Syn. Catal. 2011, 353, 1903-1907, gold-functionalized polyethylene glycol polyacrylamide copolymer (PEGA) resins, and PEG-polystyrene graft co-polymer in which the PEG chains have been terminally functionalized with an amino group (e.g. NovaSyn® TG amino resin, which is a 3000-4000 M.W.). Preferably, the gold is provided as a gold functionalized PEG-polystyrene composite resin.

According to a second aspect of the invention there is presented a first compound according to the general formula (1):

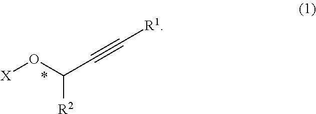

wherein R¹ and R² are independently selected from the group consisting of H, optionally substituted C₁-C₁₀ alkyl, optionally substituted C₃-C₁₀ cycloalkyl, optionally substituted C₂-C₁₀ alkenyl, optionally substituted C₃-C₁₀ cycloalkenyl, optionally substituted C₂-C₁₀ alkynyl, optionally substituted C₂-C₁₀ heteroalkyl, optionally substituted C₃-C₁₀ heterocycloalkyl, optionally substituted C₂-C₁₀ heteroalkenyl, optionally substituted C₃-C₁₀ heterocycloalkenyl, optionally substituted C₂-C₁₀ heteroalkynyl, optionally substituted C₆-C₁₄ aryl, optionally substituted C₅-C₁₄ heteroaryl,
wherein X—O comprises at least one aryl group or heteroaryl group directly connected to the oxygen (O) of the X—O substituent, and comprises an active agent or a salt thereof, and optionally comprises a linker between the oxygen and the active agent;

wherein the carbon-oxygen bond (*) is cleaved to release the active agent when the compound of formula (1) is reacted with palladium or gold.

Typically, R¹ and R² are independently selected from the group consisting of H, optionally substituted C₁-C₅ alkyl, optionally substituted C₃-C₆ cycloalkyl, optionally substituted C₂-C₆ alkenyl, optionally substituted C₃-C₆ cycloalkenyl, optionally substituted C₂-C₅ alkynyl, optionally substituted C₂-C₅ heteroalkyl, optionally substituted C₃-C₆ heterocycloalkyl, optionally substituted C₂-C₅ heteroalkenyl, optionally substituted C₃-C₆ heterocycloalkenyl, optionally substituted C₂-C₅ heteroalkynyl, optionally substituted C₆-C₁₂ aryl, optionally substituted C₅-C₁₁ heteroaryl.

For example, the first compound may have a general formula selected from the group comprising:

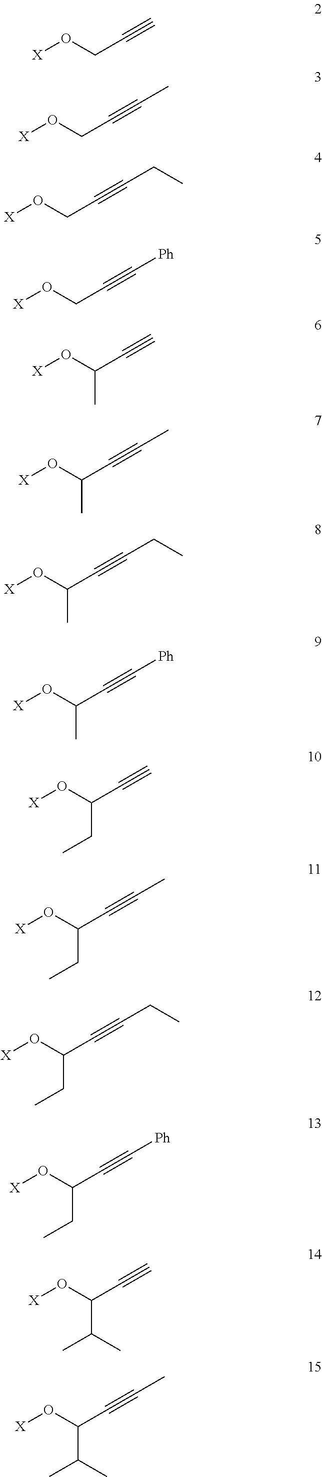

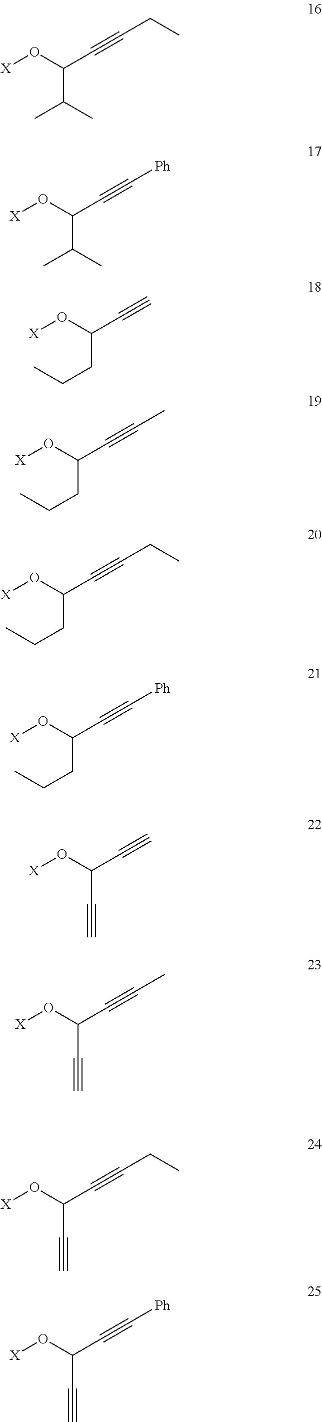

In embodiments where the X—O group comprises an active agent and a linker, the active agent may be connected to the linker via an amine, hydroxyl or carbonyl group of the active agent. The linker may comprise an aryl or heteroaryl group connecting the oxygen of the X—O group to the active agent. The linker may comprise an alkyl aryl or alkyl heteroaryl group connecting the oxygen of the X—O group to the active agent. For example, the linker may comprise an alkyl substituted benzene group, such that the linker and the oxygen of the X—O group form a alkyl phenyl group. Typically, the alkyl group is para- or ortho- to the oxygen on the aromatic ring. The linker may further comprise a carboxylic acid ester group that may be released as CO₂ when the bond (*) is cleaved by palladium or gold.

In preferred embodiments, the linker is selected from the group

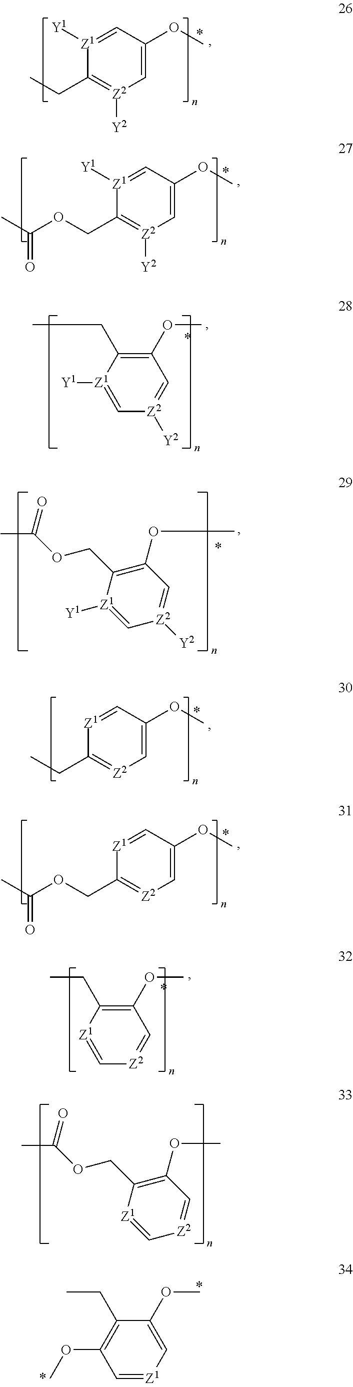

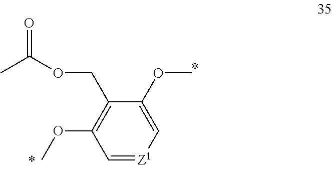

wherein
Z¹ and Z² are independently selected from N, CH, C;
Y¹ and Y² are independently selected from H, NO₂, halogen, COOR³, OR⁴;
R³ and R⁴ are independently selected from the group consisting of H, optionally substituted C₁-C₁₀ alkyl, optionally substituted C₃-C₁₀ cycloalkyl, optionally substituted C₂-C₁₀ alkenyl, optionally substituted C₃-C₁₀ cycloalkenyl, optionally substituted C₂-C₁₀ alkynyl, optionally substituted C₂-C₁₀ heteroalkyl, optionally substituted C₃-C₁₀ heterocycloalkyl, optionally substituted C₂-C₁₀ heteroalkenyl, optionally substituted C₃-C₁₀ heterocycloalkenyl, optionally substituted C₂-C₁₀ heteroalkynyl, optionally substituted C₆-C₁₄ aryl, optionally substituted C₅-C₁₄ heteroaryl; and
n is 1-10, preferably 1, 2 or 3.

In some embodiments, the first compound may comprise more than one propargyl group connected to an oxygen which is in turn connected to an aryl group. The aryl group may be part of a linker. The aryl group may be part of the active agent. For example, the first compound may comprise two, three or four propargyl-oxygen-groups. An example of an embodiment comprising two propargyl-oxygen groups is shown as Formula (36):

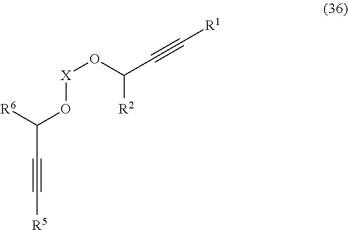

wherein R¹, R², R⁵ and R⁶ are independently selected from the group consisting of H, optionally substituted C₁-C₁₀ alkyl, optionally substituted C₃-C₁₀ cycloalkyl, optionally substituted C₂-C₁₀ alkenyl, optionally substituted C₃-C₁₀ cycloalkenyl, optionally substituted C₂-C₁₀ alkynyl, optionally substituted C₂-C₁₀ heteroalkyl, optionally substituted C₃-C₁₀ heterocycloalkyl, optionally substituted C₂-C₁₀ heteroalkenyl, optionally substituted C₃-C₁₀ heterocycloalkenyl, optionally substituted C₂-C₁₀ heteroalkynyl, optionally substituted C₆-C₁₄ aryl, optionally substituted C₅-C₁₄ heteroaryl,
and wherein O—X—O comprises at least one aryl group or heteroaryl group directly connected to each oxygen (O) of the O—X—O substituent, and comprises the active agent or a salt thereof, and optionally one or two linkers.

The same aryl group of the active agent may be directly connected to each oxygen-propargyl group. Alternatively, different aryl groups of the active agent may be directly connected to each oxygen-propargyl group.

An example of a compound according to formula (36) is

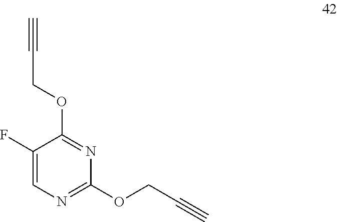

Accordingly, the first compound according to formula (1) may be selected from the following group:

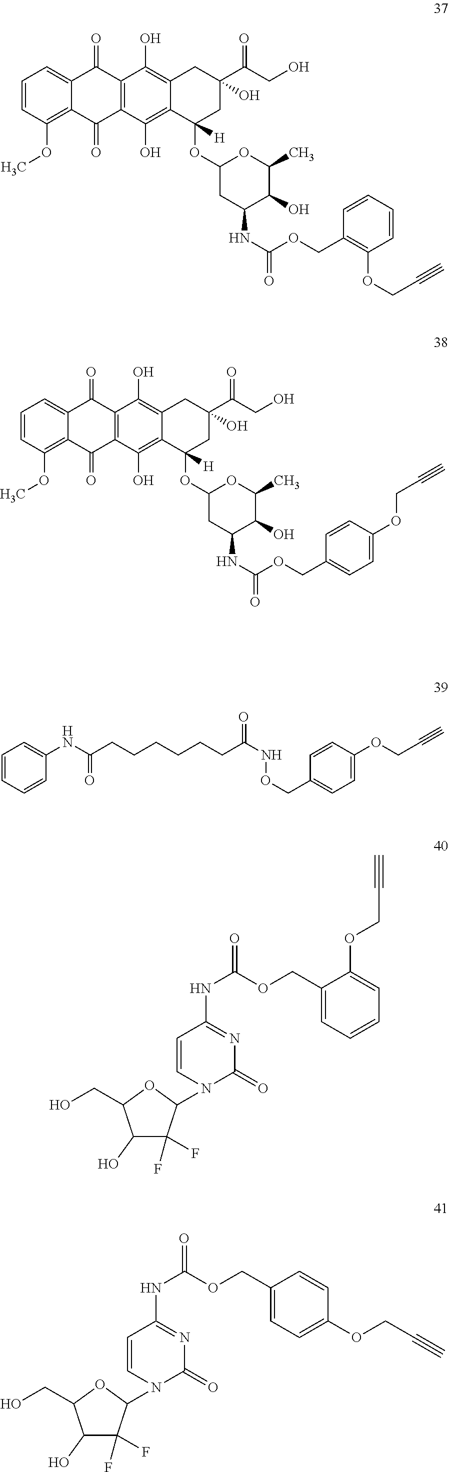 [SMILES: C#CCOc1ccccc1COC(=O)N[C@H]1CC(O[C@H]2C[C@](O)(C(=O)CO)Cc3c(O)c4c(c(O)c32)C(=O)c2c(OC)cccc2C4=O)O[C@@H](C)[C@H]1O ; C#CCOc1ccc(COC(=O)N[C@H]2CC(O[C@H]3C[C@](O)(C(=O)CO)Cc4c(O)c5c(c(O)c43)C(=O)c3c(OC)cccc3C5=O)O[C@@H](C)[C@H]2O)cc1 ; C#CCOc1ccc(CONC(=O)CCCCCCC(=O)Nc2ccccc2)cc1 ; C#CCOc1ccccc1COC(=O)Nc1ccn(C2OC(CO)C(O)C2(F)F)c(=O)n1 ; C#CCOc1ccc(COC(=O)Nc2ccn(C3OC(CO)C(O)C3(F)F)c(=O)n2)cc1]

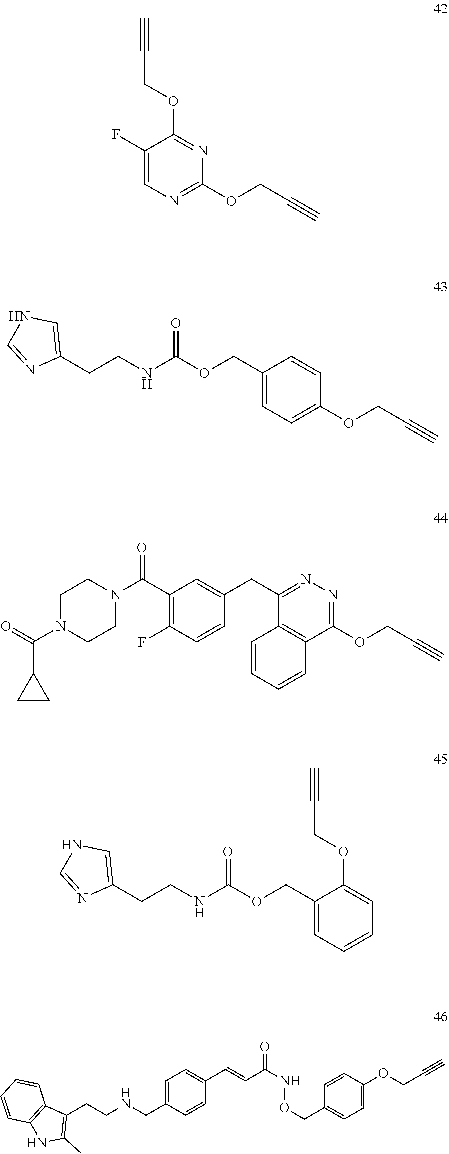 [SMILES: C#CCOc1ncc(F)c(OCC#C)n1 ; C#CCOc1ccc(COC(=O)NCCc2c[nH]cn2)cc1 ; C#CCOc1nnc(Cc2ccc(F)c(C(=O)N3CCN(C(=O)C4CC4)CC3)c2)c2ccccc12 ; C#CCOc1ccccc1COC(=O)NCCc1c[nH]cn1 ; C#CCOc1ccc(CONC(=O)/C=C/c2ccc(CNCCc3c(C)[nH]c4ccccc34)cc2)cc1]

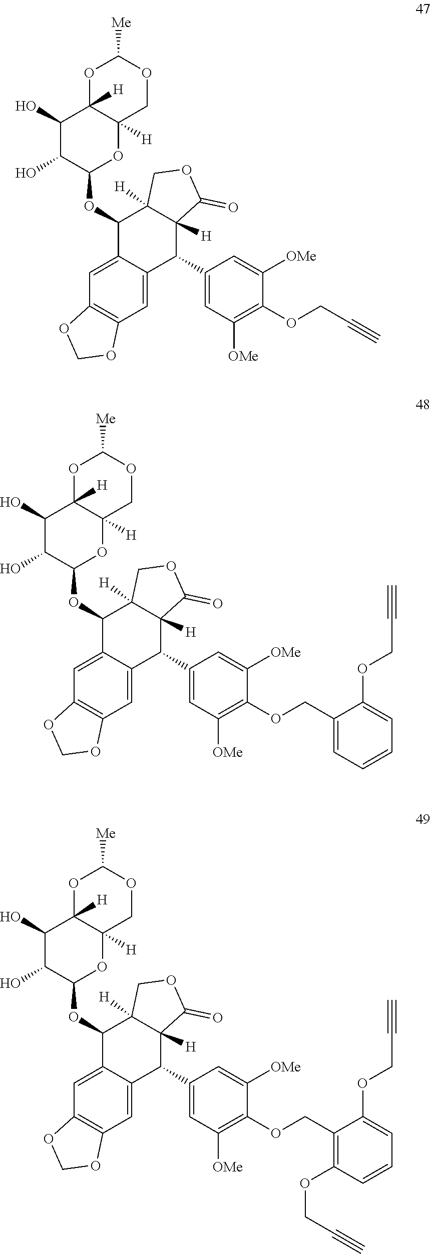

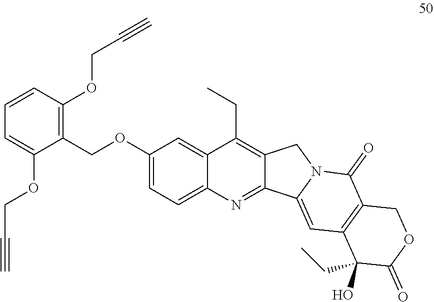 [SMILES: C#CCOc1cccc(OCC#C)c1COc1ccc2nc3c(c(CC)c2c1)Cn1c-3cc2c(c1=O)COC(=O)[C@]2(O)CC]

Further preferred and optional features of the first composition as described in the first aspect are preferred and optional features of the present aspect.

The invention extends in a third aspect to a method of treatment of disease by inserting an implant that comprises palladium and/or gold in a target area to be treated, and then delivering the first composition according to the second aspect to the target area.

The implant may comprise palladium. The implant may comprise gold. The implant may comprise both palladium and gold.

Typically the target area is an area within a subject, such as a group of cells or a section of tissue of the subject, for example. The subject may be a human patient. The subject may be a non-human animal. In some embodiments where the active agent to be released is a cytotoxic agent that is intended to treat a cancerous tumour, the target area may be the tumour to be treated or a section of the tumour to be treated, and the implant is implanted within the tumour. Accordingly, when the first compound is delivered to the target area, the first compound reacts with the palladium or gold within the implant in the tumour to release the cytotoxic active agent in the tumour.

Alternatively, the implant may be implanted near or adjacent to the target area, for example in embodiments where the target area is not readily accessible for implantation directly. The active agent may be released from the first compound near or adjacent to the target area and may diffuse into the target area.

Once the first composition has been delivered to the target area, the first composition reacts with the palladium and/or gold within the implant in the target area to release an active agent in the target area. Accordingly, the method of the present aspect allows targeted delivery of an active agent to a target area with a minimum of interaction of the active agent with the surrounding area.

According to a fourth aspect of the invention there is provided a palladium implant for use in a method of treatment, wherein the method comprises co-administering a first compound or salt according to the second aspect or a pharmaceutically acceptable salt thereof and the palladium implant to the subject.

The palladium implant may comprise palladium in particulate form. The particulate palladium may be embedded in a matrix. The matrix may fix the particulate palladium in place within the implant and thereby substantially prevents leaching or reduces the rate of leaching of the particulate palladium from the implant during use. The matrix may be a polymer matrix. The polymer matrix may be functionalised with palladium nanoparticles. The polymer matrix may comprise palladium nanoparticles embedded within it. The polymer matrix may comprise any biocompatible polymer. For example, the polymer matrix may comprise polyethyleneglycol (PEG), polystyrene, polytetrafluoroetheylene (PTFE), or expanded PTFE. The polymer matrix may comprise a co-polymer, such as a PEG-polystyrene co-polymer.

Typically the matrix is a porous matrix such that during use, the composition of the invention may diffuse into the matrix of the implant and contact the particulate palladium to initiate release of the active agent.

The particulate palladium may comprise palladium particles with an average diameter of 1 nm to 100 μm. The particulate palladium may comprise palladium particles with an average diameter of 5 nm to 1 μm. The particulate palladium may comprise palladium particles with an average diameter of 5 nm to 10 nm.

Further embodiments of the palladium implant according to the present aspect are described above. Features of the palladium implant described in the first aspect are features of the palladium implant of the present aspect.

According to a fifth aspect of the invention there is provided an implant for use in a method of treatment, wherein the method comprises administering a first compound or salt according to the second aspect or a pharmaceutically acceptable salt thereof and the implant to the subject, wherein the implant comprises palladium and/or gold.

The implant may be administered in a first step and the first compound or salt may be administered in a second step subsequent to the first. The first compound or salt may be administered in a first step and the implant may be administered in a second step subsequent to the first. Alternatively, the implant and the first compound or salt may be co-administered.

In some embodiments, the implant may comprise palladium. In some embodiments, the implant may comprise gold. In some embodiments, the implant may comprise both palladium and gold.

The implant may comprise palladium and/or gold in particulate form. The particulate palladium and/or particulate gold may be embedded in a matrix. The matrix may fix the particulate palladium and/or particulate gold in place within the implant and thereby substantially prevents leaching or reduces the rate of leaching of the particulate palladium and/or particulate gold from the implant during use. The matrix may be a polymer matrix. The polymer matrix may be functionalised with palladium nanoparticles. The polymer matrix may be functionalised with gold nanoparticles. The polymer matrix may be functionalised with both palladium nanoparticles and gold nanoparticles. The polymer matrix may comprise palladium nanoparticles embedded within it. The polymer matrix may comprise gold nanoparticles embedded within it. The polymer matrix may comprise palladium nanoparticles embedded within it and gold nanoparticles embedded within it. The polymer matrix may comprise any biocompatible polymer. For example, the polymer matrix may comprise polyethyleneglycol (PEG), polystyrene, polytetrafluoroetheylene (PTFE), or expanded PTFE. The polymer matrix may comprise a co-polymer, such as a PEG-polystyrene co-polymer.

Typically the matrix is a porous matrix such that during use, the composition of the invention may diffuse into the matrix of the implant and contact the particulate palladium and/or particulate gold to initiate release of the active agent.

In embodiments comprising particulate palladium, the particulate palladium may comprise palladium particles with an average diameter of 1 nm to 100 μm. The particulate palladium may comprise palladium particles with an average diameter of 5 nm to 1 μm. The particulate palladium may comprise palladium particles with an average diameter of 5 nm to 10 nm.

In embodiments comprising particulate gold, the particulate gold may comprise gold particles with an average diameter of 1 nm to 100 μm. The particulate gold may comprise gold particles with an average diameter of 5 nm to 1 μm. The particulate gold may comprise gold particles with an average diameter of 1 nm to 30 nm.

Further embodiments of the implant according to the present aspect are described above. Features of the implant described in the first aspect are features of the implant of the present aspect.

In a fifth aspect of the invention there is presented a kit of parts comprising the first composition of the second aspect or a pharmaceutical composition comprising the first composition of the second aspect and the implant of the fifth aspect.

Preferably, during use, the implant is implanted in a target area within a subject within which it is desired to release an active agent.

Typically, the kit is used in the method of the first aspect to release the active agent from the first compound where it comes into contact with the implant in the target area.

The invention extends in a sixth aspect to a use of the first composition of the second aspect to treat cancer.

The first composition may be used to treat any solid tumour cancer. Typically, the solid tumour may be in the target area to be treated or the target area to be treated may be at least a portion of the solid tumour. Accordingly, an implant comprising palladium and/or gold used to activate the first composition may be implanted into the solid tumour to ensure that the active agent is released from the first composition within the solid tumour to be treated.

For example, the use may be to treat cancers such as pancreatic and/or colorectal cancer, prostate cancer, ovarian cancer, breast cancer, lung cancer, liver cancer or brain cancer.

Preferably, the target area to be treated is readily accessible via surgery to allow insertion of the implant into the target area.

In a seventh aspect there is presented a pharmaceutical composition comprising the first composition of the second aspect and at least one excipient.

Chemical Groups

Halo

The term “halogen” (or “halo”) includes fluorine, chlorine, bromine and iodine.

Alkyl, Alkylene, Alkenyl, Alkynyl, Cycloalkyl etc.

The terms “alkyl”, “alkylene”, “alkenyl” or “alkynyl” are used herein to refer to both straight and branched chain acyclic forms. Cyclic analogues thereof are referred to as cycloalkyl, etc.

The term “alkyl” includes monovalent, straight or branched, saturated, acyclic hydrocarbyl groups. In one embodiment alkyl is C₁₋₁₀alkyl, in another embodiment C₁₋₆alkyl, in another embodiment C₁₋₄alkyl, such as methyl, ethyl, n-propyl, i-propyl or t-butyl groups.

The term “cycloalkyl” includes monovalent, saturated, cyclic hydrocarbyl groups. In some embodiments the cycloalkyl is C₃₋₁₀cycloalkyl, in other embodiments C₃₋₆cycloalkyl, such as cyclopentyl and cyclohexyl.

The term “alkoxy” means alkyl-O—.

The term “alkylamino” means alkyl-NH—.

The term “alkylthio” means alkyl-S(O)ₜ—, wherein t is defined below.

The term “alkenyl” includes monovalent, straight or branched, unsaturated, acyclic hydrocarbyl groups having at least one carbon-carbon double bond and, in one embodiment, no carbon-carbon triple bonds. In one embodiment alkenyl is C₂₋₁₀alkenyl, in another embodiment C₂₋₆alkenyl, in another embodiment C₂₋₄alkenyl.

The term “cycloalkenyl” includes monovalent, partially unsaturated, cyclic hydrocarbyl groups having at least one carbon-carbon double bond and, in one embodiment, no carbon-carbon triple bonds. In one embodiment cycloalkenyl is C₃₋₁₀cycloalkenyl, in another embodiment C₅₋₁₀cycloalkenyl, e.g. cyclohexenyl or benzocyclohexyl.

The term “alkynyl” includes monovalent, straight or branched, unsaturated, acyclic hydrocarbyl groups having at least one carbon-carbon triple bond and, in one embodiment, no carbon-carbon double bonds. In one embodiment, alkynyl is C₂₋₁₀alkynyl, in another embodiment C₂₋₆alkynyl, in another embodiment C₂₋₄alkynyl.

The term “alkylene” includes divalent, straight or branched, saturated, acyclic hydrocarbyl groups. In one embodiment alkylene is C₁₋₁₀alkylene, in another embodiment C₁₋₆alkylene, in another embodiment C₁₋₄alkylene, such as methylene, ethylene, n-propylene, i-propylene or t-butylene groups.

The term “alkenylene” includes divalent, straight or branched, unsaturated, acyclic hydrocarbyl groups having at least one carbon-carbon double bond and, in some embodiments, no carbon-carbon triple bonds. In some embodiments alkenylene is C₂₋₁₀alkenylene, in other embodiments C₂₋₆alkenylene, such as C₂₋₄alkenylene.

The term “cyclic group” includes carbocyclic and heterocyclic groups, such as cycloalkyl, cycloalkenyl, aryl, heterocycloalkyl, heterocycloalkenyl and heteroaryl groups as defined below.

Heterocyclic Compound

The term “heterocyclic compound” refers to a compound comprising a heterocyclic group.

The term “heterocyclic group” refers to group a saturated, partially unsaturated or unsaturated (e.g. aromatic) monocyclic or bicyclic group containing one or more (for example 1, 2, 3, 4 or 5) ring heteroatoms selected from O, S(O)ₜ or N and includes unsubstituted groups and groups substituted with one or more substituents (for example 1, 2, 3, 4 or 5 substituents), optionally wherein the one or more substituents are taken together to form a further ring system. Unless stated otherwise herein, where a heterocyclic group is bonded to another group, the heterocyclic group may be C-linked or N-linked, i.e it may be linked to the remainder of the molecule through a ring carbon atom or through a ring nitrogen atom (i.e. an endocyclic nitrogen atom). The term heterocyclic group thus includes optionally substituted heterocycloalkyl, heterocycloatkenyl and heteroaryl groups as defined below.

Heteroalkyl etc.

The term “heteroalkyl” includes alkyl groups in which up to three carbon atoms, in one embodiment up to two carbon atoms, in another embodiment one carbon atom, are each replaced independently by O, S(O)ₜ or N, provided at least one of the alkyl carbon atoms remains. The heteroalkyl group may be C-linked or hetero-linked, i.e. it may be linked to the remainder of the molecule through a carbon atom or through O, S(O)t or N, wherein t is defined below.

The term “heterocycloalkyl” includes cycloalkyl groups in which up to three carbon atoms, in one embodiment up to two carbon atoms, in another embodiment one carbon atom, are each replaced independently by O, S(O)ₜ or N, provided at least one of the cycloalkyl carbon atoms remains. Examples of heterocycloalkyl groups include oxiranyl, thiaranyl, aziridinyl, oxetanyl, thiatanyl, azetidinyl, tetrahydrofuranyl, tetrahydrothiophenyl, pyrrolidinyl, tetrahydropyranyl, tetrahydrothiopyranyl, piperidinyl, 1,4-dioxanyl, 1,4-oxathianyl, morpholinyl, 1,4-dithianyl, piperazinyl, 1,4-azathianyl, oxepanyl, thiepanyl, azepanyl, 1,4-dioxepanyl, 1,4-oxathiepanyl, 1,4-oxaazepanyl, 1,4-dithiepanyl, 1,4-thieazepanyl and 1,4-diazepanyl. The heterocycloalkyl group may be C-linked or N-linked, i.e. it may be linked to the remainder of the molecule through a carbon atom or through a nitrogen atom.

The term “heteroalkenyl” includes alkenyl groups in which up to three carbon atoms, in one embodiment up to two carbon atoms, in another embodiment one carbon atom, are each replaced independently by O, S(O)ₜ or N, provided at least one of the alkenyl carbon atoms remains. The heteroalkenyl group may be C-linked or hetero-linked, Le. it may be linked to the remainder of the molecule through a carbon atom or through O, S(O)ₜ or N.

The term “heterocycloalkenyl” includes cycloalkenyl groups in which up to three carbon atoms, in one embodiment up to two carbon atoms, in another embodiment one carbon atom, are each replaced independently by O, S(O)ₜ or N, provided at least one of the cycloalkenyl carbon atoms remains. Examples of heterocycloalkenyl groups include 3,4-dihydro-2H-pyranyl, 5-6-dihydro-2H-pyranyl, 2H-pyranyl, 1,2,3,4-tetrahydropyridinyl and 1,2,5,6-tetrahydropyridinyl. The heterocycloalkenyl group may be C-linked or N-linked, i.e. it may be linked to the remainder of the molecule through a carbon atom or through a nitrogen atom.

The term “heteroalkynyl” includes alkynyl groups in which up to three carbon atoms, in one embodiment up to two carbon atoms, in another embodiment one carbon atom, are each replaced independently by O, S(O)ₜ or N, provided at least one of the alkynyl carbon atoms remains. The heteroalkynyl group may be C-linked or hetero-linked, i.e. it may be linked to the remainder of the molecule through a carbon atom or through O, S(O)ₜ or N.

The term “heteroalkylene” includes alkylene groups in which up to three carbon atoms, in one embodiment up to two carbon atoms, in another embodiment one carbon atom, are each replaced independently by O, S(O)ₜ or N, provided at least one of the alkylene carbon atoms remains.

The term “heteroalkenylene” includes alkenylene groups in which up to three carbon atoms, in one embodiment up to two carbon atoms, in another embodiment one carbon atom, are each replaced independently by O, S(O)ₜ or N, provided at least one of the alkenylene carbon atoms remains.

Aryl

The term “aryl” includes monovalent, aromatic, cyclic hydrocarbyl groups, such as phenyl or naphthyl (e.g. 1-naphthyl or 2-naphthyl). In general, the aryl groups may be monocyclic or polycyclic fused ring aromatic groups. Preferred aryl refers to C₅-C₁₄aryl. Other examples of aryl groups are monovalent derivatives of aceanthrylene, acenaphthylene, acephenanthrylene, anthracene, azulene, chrysene, coronene, fluoranthene, fluorene, as-indacene, s-indacene, indene, naphthalene, ovalene, perylene, phenalene, phenanthrene, picene, pleiadene, pyrene, pyranthrene and rubicene.

The term “arylalkyl” means alkyl substituted with an aryl group, e.g. benzyl.

Heteroaryl

The term “heteroaryl” includes aryl groups in which one or more carbon atoms are each replaced by heteroatoms independently selected from O, S, N and NR^{N}, where R^{N} is defined below (and in one embodiment is H or alkyl (e.g. C₁₋₆alkyl)),

In general, the heteroaryl groups may be monocyclic or polycyclic (e.g. bicyclic) fused ring heteroaromatic groups. Typically, heteroaryl groups contain 5-14 ring members (preferably 5-10 members) wherein 1 2, 3 or 4 ring members are independently selected from O, S, N and NR^{N}. In one embodiment, a heteroaryl group may be 5, 6, 9 or 10 membered, e.g. 5-membered monocyclic, 6-membered monocyclic, 9-membered fused-ring bicyclic or 10-membered fused-ring bicyclic.

Monocyclic heteroaromatic groups include heteroaromatic groups containing 5-6 ring members wherein 1, 2, 3 or 4 ring members are independently selected from O, S, N or NR^{N}.

In one embodiment, 5-membered monocyclic heteroaryl groups contain 1 ring member which is an —NR^{N}— group, an —O— atom or an —S— atom and, optionally, 1-3 ring members (e.g. 1 or 2 ring members) which are ═N— atoms (where the remainder of the 5 ring members are carbon atoms).

Examples of 5-membered monocyclic heteroaryl groups are pyrrolyl, furanyl, thiophenyl, pyrazolyl, imidazolyl, Isoxazolyl, oxazolyl, isothiazolyl, thiazolyl, 1,2,3 triazolyl, 1,2,4triazolyl, 1,2,3 oxadiazolyl, 1,2,4 oxadiazolyl, 1,2,5 oxadiazolyl, 1,3,4 oxadiazolyl, 1,3,4thiadiazolyl, pyridyl, pyrimidinyl, pyridazinyl, pyrazinyl, 1,3,5 triazinyl, 1,2,4 triazinyl, 1,2,3 triazinyl and tetrazolyl.

Examples of 6-membered monocyclic heteroaryl groups are pyridinyl, pyridazinyl, pyrimidinyl and pyrazinyl.

In one embodiment, 6-membered monocyclic heteroaryl groups contain 1 or 2 ring members which are ═N— atoms (where the remainder of the 6 ring members are carbon atoms).

Bicyclic heteroaromatic groups include fused-ring heteroaromatic groups containing 9-14 ring members wherein 1, 2, 3, 4 or more ring members are independently selected from O, S, N or NR^{N}.

In one embodiment, 9-membered bicyclic heteroaryl groups contain 1 ring member which is an —NR^{N}— group. an —O— atom or an —S— atom and, optionally, 1-3 ring members (e.g. 1 or 2 ring members) which are ═N— atoms (where the remainder of the 9 ring members are carbon atoms).

Examples of 9-membered fused-ring bicyclic heteroaryl groups are benzofuranyl, benzothiophenyl, indolyl, benzimidazolyl, indazolyl, benzotriazolyl, pyrrolo[2,3-b]pyridinyl, pyrrolo[2,3-c]pyridinyf. pyrrolo[3,2-c]pyridinyl, pyrrolo[3,2-b]pyridinyl, imidazo[4,5-b]pyridinyl, imidazo[4,5-c]pyridinyl, pyrazolo[4,3-d]pyridinyl, pyrazolo[4,3-c]pyridinyl, pyrazolo[3,4-c]pyridinyl, pyrazolo[3,4-b]pyridinyl, isoindolyl, indazolyl, purinyl, indolininyl, imidazo[1,2-a]pyridinyl, imidazo[1,5-a]pyridinyl, pyrazolo[1,2-a]pyridinyl, pyrrolo[1,2-b]pyridazinyi and imidazo[1,2-c]pyrimidinyl.

In one embodiment, 10-membered bicyclic heteroaryl groups contain 1-3 ring members which are ═N— atoms (where the remainder of the 10 ring members are carbon atoms).

Examples of 10-membered fused-ring bicyclic heteroaryl groups are quinolinyl, isoquinolinyl, cinnolinyl, quinazolinyl, quinoxalinyl, phthalazinyl, 1,6-naphthyridinyl, 1,7-naphthyridinyl, 1,8-naphthyridinyl, 1,5-naphthyridinyl, 2,6-naphthyridinyl, 2,7-naphthyridinyl, pyrido[3,2-d]pyrimidinyl, pyrido[4,3-d]pyrimidinyl, pyrido[3,4-d]pyrimidinyl, pyrido[2,3-c]pyrimidinyl, pyrido[2,3-b]pyrazinyl, pyrido[3,4-b]pyrazinyl, pyrimido[5,4-d]pyrimidinyl, pyrazino[2,3-b]pyrazinyl and pyrimido[4,5-d]pyrimidinyl.

The term “heteroarylalkyl” means alkyl substituted with a heteroaryl group

The term “nucleobase” refers to a compound containing a base according to any nucleoside, such as an adenine, guanine, cytosine, thymine and uracil.

The term analog or derivative refers to compounds that have a close structural and, preferably, functional similarity to a given reference compound.

General

Unless indicated explicitly otherwise, where combinations of groups are referred to herein as one moiety, e.g. arylalkyl, the last mentioned group contains the atom by which the moiety is attached to the rest of the molecule.

Where reference is made to a carbon atom of an alkyl group or other group being replaced by O, S(O)ₜ or N, what is intended is that:

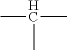

is replaced by

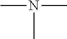

—CH═ is replaced by —N═;

═C—H is replaced by ═N; or

—CH2- is replaced by —O—, —S(O), or —NR^{N}—.

By way of clarification, in relation to the above mentioned heteroatom containing groups (such as heteroalkyl etc.), where a numerical of carbon atoms is given, for instance C₃₋₅heteroalkyl, what is intended is a group based on C₃₋₆alkyl in which one of more of the 3-6 chain carbon atoms is replaced by O, S(O)ₜ or N. Accordingly, a C₃₋₆heteroalkyl group, for example, will contain less than 3-6 chain carbon atoms.

Where mentioned above, R^{N} is H, alkyl, cycloalkyl, aryl, heteroaryl, —C(O)-alkyl, —C(O)-aryl, —C(O)-heteroaryl, —S(O)ₜ-alkyl, —S(O)ₜ-aryl or —S(O)ₜ-heteroaryl. R^{N} may, in particular, be H, alkyl (e.g. C₁₋₆alkyl) or cycloalkyl (e.g. C₃₋₅cycloalkyl).

Where mentioned above, t is independently 0, 1 or 2, for example 2. Typically, t is 0.

Where a group has at least 2 positions which may be substituted, the group may be substituted by both ends of an alkylene or heteroalkylene chain to form a cyclic moiety.

Substituents

Optionally substituted groups of the compounds of the invention (e.g. heterocyclic groups, alkyl, cycloalkyl, alkoxy, alkenyl, cycloalkenyl, alkynyl, alkylene, alkenylene, heteroalkyl, heterocycloalkyt, heteroalkenyl, heterocycloalkenyl, heteroalkynyl, heteroalkylene, heteroalkenylene, aryl, arylalkyl, arylheteroalkyl, heteroaryl, heteroarylalkyl or heteroarylheteroalkyl groups etc.) may be substituted or unsubstituted, in one embodiment unsubstituted. Typically, substitution involves the notional replacement of a hydrogen atom with a substituent group, or two hydrogen atoms in the case of substitution by ═O.

Where substituted, there will generally be 1 to 3 substituents unless otherwise stated herein, in one embodiment 1 or 2 substituents, for example 1 substituent.

The optional substituent(s) may be selected independently from the groups consisting of halogen, trihalomethyl, trihaloethyl, OH, NH₂, —NO₂, —CN, —N⁺(C₁₋₆alkyl)₂O⁻, —CO₂H, —CO₂C₁₋₆alkyl, —SO₃H, —SOC₁₋₆alkyl, —SO₂C₁₋₆alkyl, —SO₃C₁₋₆alkyl, —OC(═O)OC₁₋₆alkyl, —C(═O)H, —C(═O)C₁₋₆alkyl, —OC(═O)C₁₋₆alkyl, ═O, —N(C₁₋₆alkyl)₂, —C(═O)NH₂, —C(═O)N(C₁₋₆alkyl)₂, —N(C₁₋₆alkyl)C(═O)O(C₁₋₆alkyl), —N(C₁₋₆alkyl)C(═O)N(C₁₋₆alkyl)₂, —OC(═O)N(C₁₋₆alkyl)₂, —N(C₁₋₆alkyl)C(═O)C₁₋₆alkyl, —C(═S)N(C₁₋₆alkyl)₂, —N(C₁₋₆alkyl)C(═S)C₁₋₆alkyl, —SO₂N(C₁₋₆alkyl)₂, —N(C₁₋₆alkyl)SO₂C₁₋₆alkyl, —N(C₁₋₆alkyl)C(═S)N(C₁₋₆alkyl)₂, —N(C₁₋₆)SO₂N(C₁₋₆alkyl)₂, —C₁₋₆alkyl, —C₁₋₆heteroalkyl, —C₃₋₆cycloalkyl, —C₃₋₆heterocycloalkyl, —C₂₋₆alkenyl, —C₂₋₆heteroalkenyl, —C₃₋₆cycloalkenyl, —C₃₋₆heterocycloalkenyl, —C₂₋₆alkynyl, —C₂₋₆heteroalkynyl, —Z^{u}—C₁₋₆alkyl, —Z^{u}—C₃₋₆cycloalkyl , —Z^{u}—C₂₋₆alkenyl, —Z^{u}—C₃₋₆cycloalkenyl and —Z^{u}—C₂₋₆alkynyl, wherein
Z^{u} is independently O, S, NH or N(C₁₋₆alkyl).

In another embodiment, the optional substituent(s) is/are independently OH, NH₂, halogen, trihalomethyl, trihaloethyl, —NO₂, —CN, —N⁺(C₁₋₆alkyl)₂O⁻, —CO₂H, —SO₃H, —SOC₁₋₆alkyl, —SO₂C₁₋₆alkyl, —C(═O)H, —C(═O)C₁₋₆alkyl, ═O, —N(C₁₋₆alkyl)₂, —C(═O)NH₂, —C₁₋₆alkyl, —C₃₋₆cycloalkyl, —C₃₋₆heterocycloalkyl, —Z^{u}C₁₋₆alkyl or —Z^{u}—C₃₋₆cycloalkyl, wherein Z^{u} is defined above.

In another embodiment, the optional substituent(s) is/are independently OH, NH₂, halogen, trihalomethyl, —NO₂, —CN, —CO₂H, —C(═O)C₁₋₆alkyl, ═O, —N(C₁₋₆alkyl)₂, —C(═O)NH₂, —C₁₋₆alkyl, —C₃₋₆cycloalkyl, —C₃₋₆heterocycloalkyl, —Z^{u}C₁₋₆alkyl or —Z^{u}—C₃₋₆cycloalkyl, wherein Z^{u} is defined above.

In another embodiment, the optional substituent(s) is/are independently halogen, OH, NH₂, —NO₂, —CN, —CO₂H, ═O, —N(C₁₋₆alkyl)₂, —C₁₋₆alkyl, —C₃₋₆cycloalkyl or —C_{3.6}heterocycloalkyl.

In another embodiment, the optional substituent(s) is/are independently halogen, OH, NH₂, ═O, —C₁₋₆alkyl, —C₃₋₆cycloalkyl or —C₃₋₆heterocycloalkyl.

Compounds of the Invention and Derivatives Thereof

As used herein, the terms “compounds of the invention” and “compound of formula (1)” etc. include pharmaceutically acceptable derivatives thereof and polymorphs, isomers and isotopically labelled variants thereof.

Pharmaceutically Acceptable Derivatives

The term “pharmaceutically acceptable derivative” includes any pharmaceutically acceptable salt, solvate, hydrate or prodrug of a compound of the invention. In one embodiment, the pharmaceutically acceptable derivatives are pharmaceutically acceptable salts, solvates or hydrates of a compound of the invention, particularly pharmaceutically acceptable salts.

Pharmaceutically Acceptable Salts

Salts of the compounds of the invention may be formed where acidic or basic groups are present. In typical embodiments the salts are pharmaceutically acceptable salts.

Compounds of the invention which contain basic, e.g. amino, groups are capable of forming salts, such as pharmaceutically acceptable salts, with acids. In embodiments, pharmaceutically acceptable acid addition salts of the compounds of the invention include salts of inorganic acids such as hydrohalic acids (e.g. hydrochloric, hydrobromic and hydroiodic acid), sulfuric acid, nitric acid and phosphoric acids. In embodiments, pharmaceutically acceptable acid addition salts of the compounds of the invention include those of organic acids such as aliphatic, aromatic, carboxylic and sulfonic classes of organic acids, examples of which include: aliphatic monocarboxylic acids such as formic acid, acetic acid, propionic acid and butyric acid; aliphatic hydroxy acids such as lactic acid, citric acid, tartaric acid and malic acid; dicarboxylic acids such as maleic acid and succinic acid; aromatic carboxylic acids such as benzoic acid, p-chlorobenzoic acid, phenylacetic acid, diphenylacetic acid and triphenylacetic acid; aromatic hydroxyl acids such as o-hydroxybenzoic acid, p-hydroxybenzoic acid, 1-hydroxynaphthalene-2-carboxylic acid and 3-hydroxynaphthalene-2-carboxylic acid; and sulfonic acids such as methanesulfonic acid, ethanesulfonic acid and benzenesulfonic acid. Other pharmaceutically acceptable acid addition salts of the compounds of the invention include those of glycolic acid, glucuronic acid, furoic acid, glutamic acid, anthranilic acid, salicylic acid, mandelic acid, embonic (pamoic) acid, pantothenic acid, stearic acid, sulfanilic acid, algenic acid and galacturonic acid. Wherein the compound of the invention comprises a plurality of basic groups, multiple centres may be protonated to provide multiple salts, e.g. di- or tri-salts of compounds of the invention.

For example, a hydrohalic acid salt of a compound of the invention as described herein may be a monohydrohalide, dihydrohalide or trihydrohalide, etc. In one embodiment, the salts include, but are not limited to those resulting from addition of any of the acids disclosed above. In one embodiment of the compound of the invention, two basic groups form acid addition salts. In a further embodiment, the two addition salt counterions are the same species, e.g. dihydrochloride, dihydrosulphide etc. Typically, the pharmaceutically acceptable salt is a hydrochloride salt, such as a dihydrochloride salt.

Compounds of the invention which contain acidic, e.g. carboxyl, groups are capable of forming pharmaceutically acceptable salts with bases. Pharmaceutically acceptable basic salts of the compounds of the invention include, but are not limited to, metal salts such as alkali metal or alkaline earth metal salts (e.g. sodium, potassium, magnesium or calcium salts) and zinc or aluminium salts, and salts formed with ammonia, organic amines (e.g. ammonium, mono-, di-, tri- and tetraalkylammonium salts), or heterocyclic bases such as ethanolamines (e.g. diethanolamine), benzylamines, N-methyl-glucamine, and amino acids (e.g. lysine). In typical embodiments, the base addition salt is selected from sodium, potassium and ammonium, mono-, di-, tri- and tetraalkylammonium salts. In one embodiment, pharmaceutically acceptable basic salts of the compounds of the invention include, but are not limited to, salts formed with ammonia or pharmaceutically acceptable organic amines or heterocyclic bases such as ethanolamines (e.g. diethanolamine), benzylamines, N-methyl-glucamine, and amino acids (e.g. lysine).

Hemisalts of acids and bases may also be formed, e.g. hemisulphate salts.

Pharmaceutically acceptable salts of compounds of the invention may be prepared by methods well-known in the art.

For a review of pharmaceutically acceptable salts, see Stahl and Wermuth, Handbook of Pharmaceutical Salts: Properties, Selection and Use (Wiley-VCH, Weinheim, Germany, 2002).

Solvates & Hydrates

The compounds of the invention may exist in both unsolvated and solvated forms. The term “solvate” includes molecular complexes comprising a compound of the invention and one or more pharmaceutically acceptable solvent molecules such as water or C₁₋₆ alcohols, e.g. ethanol. The term “hydrate” means a “solvate” where the solvent is water.

Prodrugs

The compounds of the present invention act as bioorthogonal prodrugs which may be cleaved in the presence of palladium or gold. However, the compounds of the invention may be used with conventional prodrug strategies and thus may further include pro-moieties which are, when administered in vivo, converted into compounds of the invention (e.g. compounds of formula (1)) under biological conditions Tegafur is for example a known prodrug of 5-FU. Thus, the invention provides compounds of the invention wherein the heterocyclic compound is tegafur, i.e. wherein the X—O group according to formula (1) comprises a tegafur residue.

Suitable pro-moieties for use alongside the propargyl groups of formula (1) in the compounds of the invention are metabolized in vivo to form a compound of the invention comprising the first compound of formula (1) or an active agent is produced when the propargyl group is cleaved from the X—O group of formula (1) in the presence of palladium or gold. The design of prodrugs is well-known in the art, as discussed in Bundgaard, Design of Prodrugs 1985 (Elsevier), The Practice of Medicinal Chemistry 2003, 2nd Ed, 561-585 and Leinweber, Drug Metab. Res. 1987, 18: 379.

Examples of prodrugs of compounds of the invention are esters and amides of the compounds of the invention (e.g. esters and amides of compounds of formula (1)). For example, where the compound of the invention contains a carboxylic acid group (—COOH), the hydrogen atom of the carboxylic acid group may be replaced in order to form an ester (e.g. the hydrogen atom may be replaced by C₁₋₆alkyl). Where the compound of the invention contains an alcohol group (—OH), the hydrogen atom of the alcohol group may be replaced in order to form an ester (e.g. the hydrogen atom may be replaced by —C(O)C₁₋₆alkyl. Where the compound of the invention contains a primary or secondary amino group, one or more hydrogen atoms of the amino group may be replaced in order to form an amide (e.g. one or more hydrogen atoms may be replaced by —C(O)C₁₋₆alkyl).

Amorphous & Crystalline Forms

The compounds of the invention may exist in solid states from amorphous through to crystalline forms. All such solid forms are included within the invention.

Isomeric Forms

Compounds of the invention may exist in one or more geometrical, optical, enantiomeric, diastereomeric and tautomeric forms, including but not limited to cis- and trans-forms, E- and Z-forms, R-, S- and meso-forms, keto- and enol-forms. All such isomeric forms are included within the invention. The isomeric forms may be in isomerically pure or enriched form, as well as in mixtures of isomers (e.g. racemic or diastereomeric mixtures).

### BRIEF DESCRIPTION OF THE FIGURES

Embodiments of the present invention will now be described, by way of non-limiting example, with reference to the accompanying drawings.

FIG. 1 shows the relationship between active agent/prodrug concentrations for Vorinostat and Vorinostat prodrugs and cell viability (%) for lung cancer A549 cells (FIG. 1A), glioblastoma U87G cells (FIG. 1B) and glioblastoma T98 cells (FIG. 1C) and the respective calculated EC₅₀ values.

FIG. 2 shows High Performance Liquid Chromatography (HPLC) chromatograms (UV detector 254 nm) of Pd⁰-mediated conversion of propargyloxybenzyl-Vorinostat (POB-Vor) into Vorinostat performed in phosphate buffered saline (PBS, pH=7.3) and incubated for 24 h at 37° C. (Thermomixer, shaker speed: 1,200 rpm). The HPLC chromatograms for the conversion of POB-Vor to Vorinostat at 0 h(A), 3 h(B) and 6 h(C).

FIG. 3 shows the results of a BOOM conversion study showing relative toxicities (as indicated by % cell viability) against A549 cells of prodrug-palladium combinations for POB-Vor and Benzyl-Vor compared to Vorinostat.

FIG. 4 shows the dose dependent toxicology data (bar graph) indicated by % cell viability (A549 cells in FIG. 4A, U87G cells in FIG. 4B, and T98 cells in FIG. 4C) for conversion of POB-Vor into Vorinostat using extracellular palladium resins.

FIG. 5 shows the phase-contrast images of A549 cells after 5 days of treatment (120 h). Cell proliferation was monitored using the high-content live-cell imaging system Incucyte™ (Essen BioScience) placed in an incubator (5% CO₂, 37° C.). POB-Vor and Vorinostat were used at 100 μM.

FIG. 6 shows the relationship between active agent/prodrug concentrations for Doxorubicin and Doxorubicin prodrugs and cell viability (%) for lung cancer A549 cells (FIG. 6A), prostate cancer DU145 cells (FIG. 6B) and glioblastoma T98 cells (FIG. 6C) and the respective calculated EC₅₀ values.

FIG. 7 shows the results of a BOOM conversion study showing relative toxicities (as indicated by % cell viability) against A549 cells (FIG. 7A), DU145 cells (FIG. 7B) and T98 cells (FIG. 7C) of prodrug-palladium combinations for oPOBC-Dox, pPOBC-Dox or Cbz-Dox compared to Doxorubicin.

FIG. 8 shows the dose dependent toxicology data (bar graph) indicated by % cell viability (A549 cells in FIG. 8A, DU145 cells in FIG. 8B and T98 cells in FIG. 8C) for conversion of Doxorubicin prodrugs into Doxorubicin using extracellular palladium resins.

FIG. 9 shows the relationship between active agent/prodrug concentrations for Gemcitabine and Gemcitabine prodrugs and cell viability (%) for pancreatic cancer MiaPaCa2 cells and the respective calculated EC₅₀ values.

FIG. 10 shows the results of a BOOM conversion study showing relative toxicities (as indicated by % cell viability) against MiaPaCa2 cells of prodrug-palladium combinations for pPOBC-Gem or Cbz-Gem compared to Gemcitabine.

FIG. 11 shows the dose dependent toxicology data (bar graph) indicated by % cell viability (MiaPaCa2 cells) for conversion of pPOBC-Gem into Gemcitabine using extracellular palladium resins.

FIG. 12 shows the results of Ninhydrin test after incubation of histamine dihydrochloride (Hist), oPOBC-Hist and pPOBC-Hist in PBS with Pd⁰-functionalized resin at 37° C. for 24 h (Thermomixer, shaker speed: 1,200 rpm).

FIG. 13 shows Liquid Chromatography-Mass Spectroscopy (LCMS) chromatograms (microTOF II detector) of oPOBC-Hist incubated with Pd⁰-resins in PBS at 37° C. for 24 h (Thermomixer, shaker speed: 1,200 rpm). FIGS. 13A, 13B and 13C show the chromatograms at 0 h, 3 h and 6 h, respectively.

FIG. 14 shows LCMS chromatograms (microTOF II detector) of pPOBC-Hist incubated with Pd⁰-resins in PBS at 37° C. for 24 h (Thermomixer, shaker speed: 1,200 rpm). FIGS. 14A, 14B and 14C show the chromatograms at 0 h, 3 h and 6 h, respectively.

FIG. 15 shows the relationship between active agent/prodrug concentrations for 5-FU and 5-FU prodrug and cell viability (%) for pancreatic BxPC3 (FIG. 15A) and colorectal HCT116 cells (FIG. 15B) and the respective calculated EC₅₀ values.

FIG. 16 shows the results of a BOOM conversion study showing relative toxicities (as indicated by % cell viability) against BxPC3 cells (FIG. 16A) and HCT116 cells (FIG. 16B) of prodrug-palladium combinations for bis-Pro-5-FU compared to 5-FU.

FIG. 17 shows the dose dependent toxicology data (bar graph) indicated by % cell viability (BxPC3 cells in FIG. 17A, HCT116 cells in FIG. 17B) for conversion of bis-Pro-5-FU into 5-FU using extracellular palladium resins.

FIG. 18 shows the relationship between drug/prodrug concentrations for Olaparib and Olaparib prodrug and cell viability (%) for ovarian A2780 cells and the respective calculated EC₅₀ values.

FIG. 19 shows the relationship between active agent/prodrug concentrations for Panobinostat prodrug and cell viability (%) for lung cancer A549 cells (FIG. 19A) and the respective calculated EC₅₀ values. FIG. 19B shows the results of a BOOM conversion study showing relative toxicities (as indicated by % cell viability) against A549 cells of prodrug-palladium combinations for POB-Panob compared to Panobinostat.

FIG. 20 shows the relationship between active agent/prodrug concentrations for SN-38 prodrug and cell viability (%) for glioblastoma U87G cells (FIG. 20A) and the respective calculated EC₅₀ values. FIG. 20B shows the results of a BOOM conversion study showing relative toxicities (as indicated by % cell viability) against glioblastoma U87G cells of prodrug-palladium combinations for di-oPOB-SN-38 compared to SN-38.

FIG. 21 shows the relationship between active agent/prodrug concentrations for the Etoposide prodrug and cell viability (%) for glioblastoma U87G cells (FIG. 20A) and the respective calculated EC₅₀ values.

FIG. 22 shows the results of a BOOM conversion study showing relative toxicities (as indicated by % cell viability) against A549 cells of prodrug and gold or palladium-gold resins combinations for POB-Vor compared to Vorinostat.

FIG. 23 shows the results of a BOOM conversion study showing relative toxicities (as indicated by % cell viability) against A549 cells of prodrug and gold or palladium-gold resins combinations for POB-Panob compared to Panobinostat.

### DETAILED DESCRIPTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

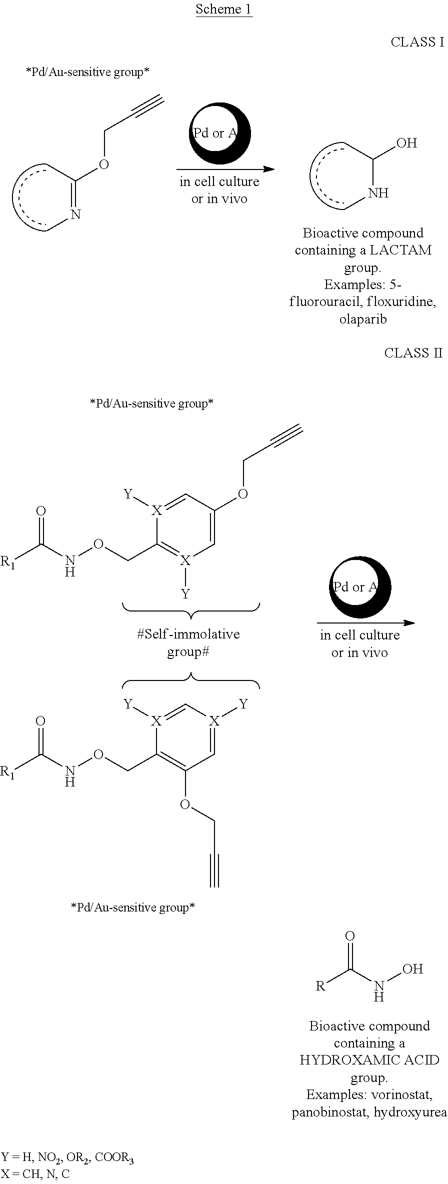

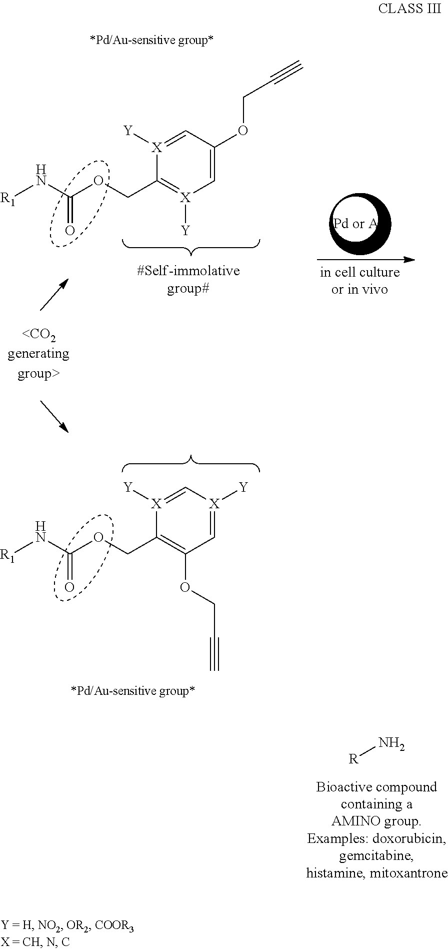

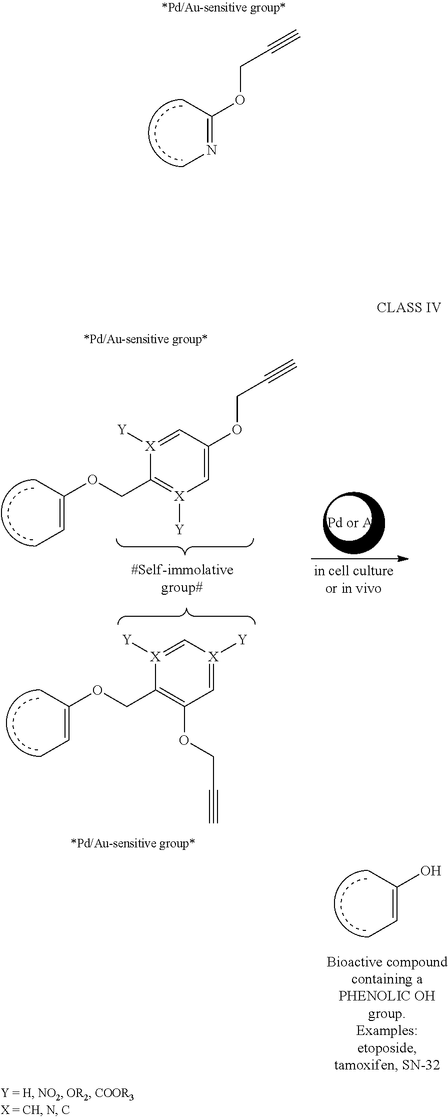

Hydroxamic Acids—Vorinostat (“Class II”)

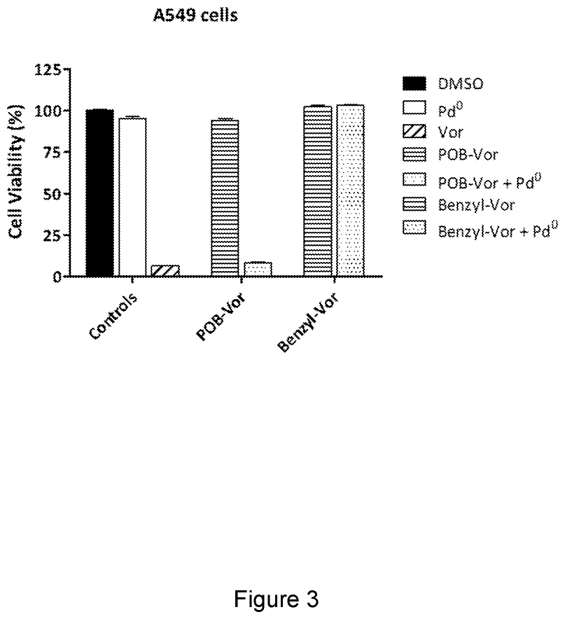

Cell viabilities for each prodrug provided as a combination with palladium is presented in FIG. 3 and gold is presented in FIG. 33 in the right of each set of two bars. Data for prodrug in the absence of palladium or gold catalyst is also provided (the left bar of each set of two bars) for comparison along with negative controls (from left to right: DMSO, Pd⁰ or Au and Vorinostat). Cells were incubated in tissue culture media containing 0.1% (v/v) DMSO and: Pd⁰, Au, or Pd/Au-resins (1 mg/mL, negative control); 100 μM of each prodrug (negative control); and Pd⁰, Au, or Pd/Au-resins (1 mg/mL)+100 μM of each prodrug (BOOM reaction assay). Cells incubated in 0.1% (v/v) DMSO in media were used as untreated cell control.

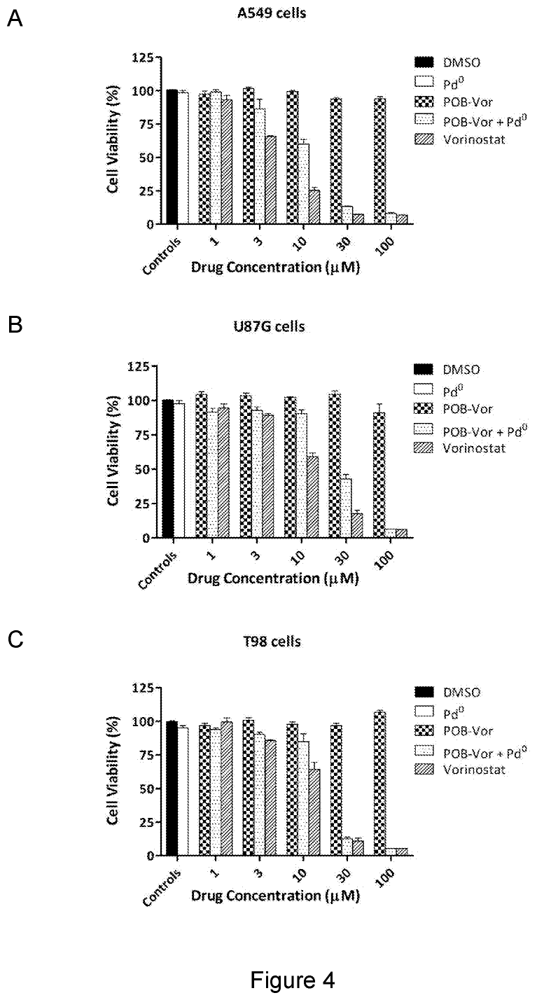

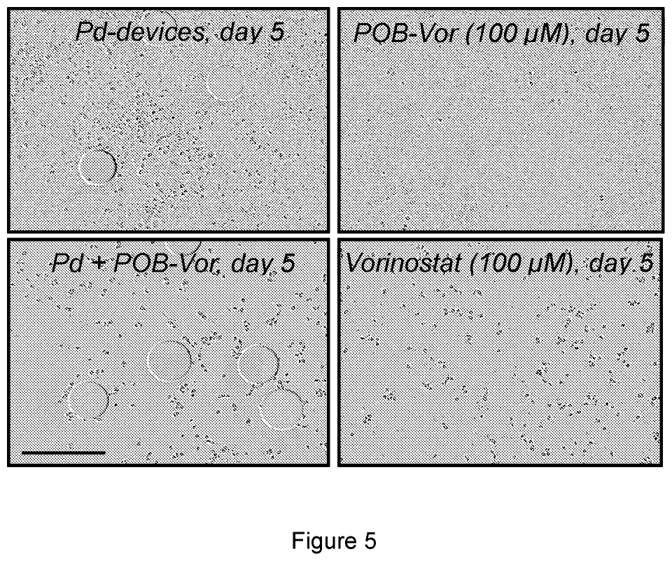

Following 5 days treatment, cells were incubated with PrestoBlue™ Cell Viability Reagent (Life Technologies) for 60-90 min. Fluorescence intensity values were related to the untreated cells (100% cell viability). Data are provided in FIG. 4 for cells incubated in tissue culture media containing 0.1% (v/v) DMSO and: Pd⁰-resins (0.8 mg/mL for U87G cells and 1 mg/mL for A549 and T98 cells, negative control); 1-100 μM of POB-Vor (negative control); 1-100 μM of Vorinostat (positive control); and Pd⁰-resin (0.8 mg/mL for U87G cells and 1 mg/mL for A549 and T98 cells)+POB-Vor (BOOM reaction assay). Cells incubated in 0.1% (v/v) DMSO in media were used as untreated cell control.

Carbamates—Doxorubicin (“Class III”)

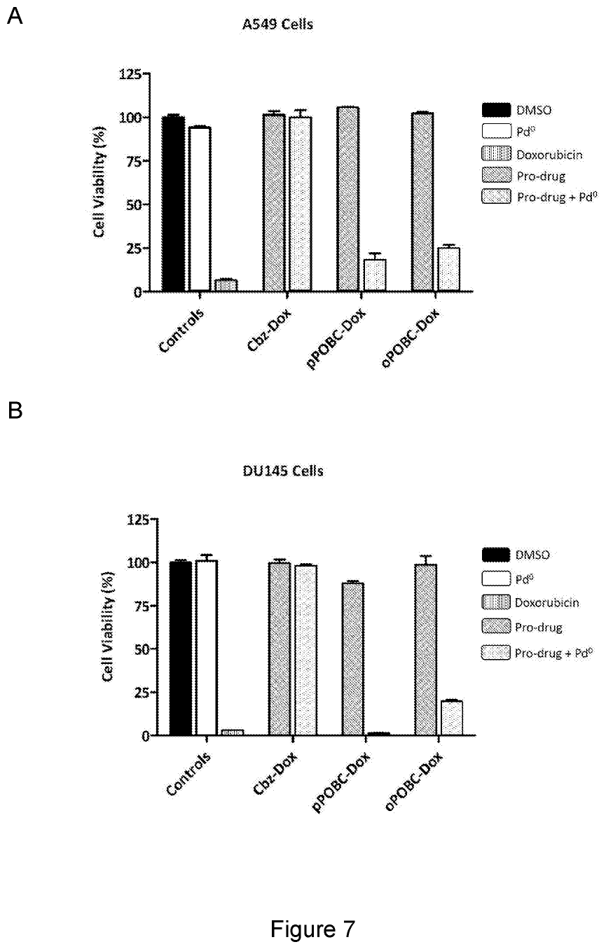

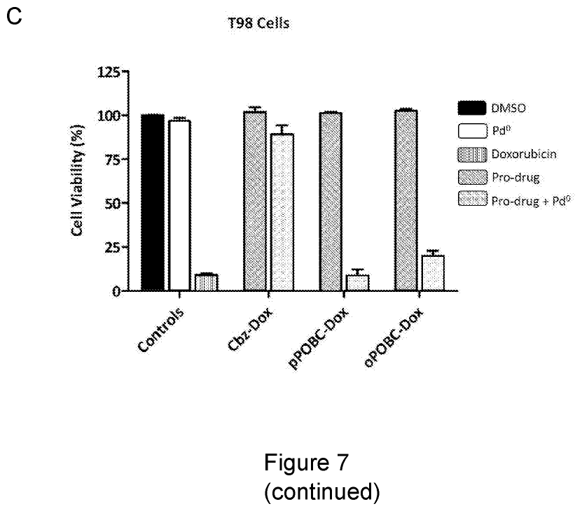

Cell viabilities for each prodrug provided as a combination with palladium is presented in FIG. 7 in the right of each set of two bars. Data for prodrug in the absence of palladium catalyst is also provided (the left bar of each set of two bars) for comparison along with negative controls (from left to right: DMSO, Pd⁰ and Doxorubicin). Cells were incubated in tissue culture media containing 0.1% (v/v) DMSO and: Pd⁰-resins (1 mg/mL, negative control); 1 μM of each prodrug (negative control); and Pd⁰-resins (1 mg/mL)+1 μM of each prodrug (BOOM reaction assay). Cells incubated in 0.1% (v/v) DMSO in media were used as untreated cell control.

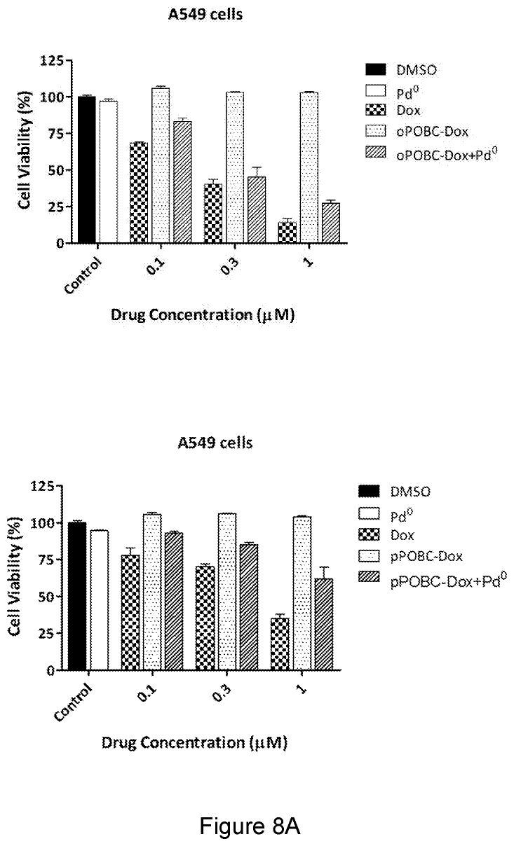

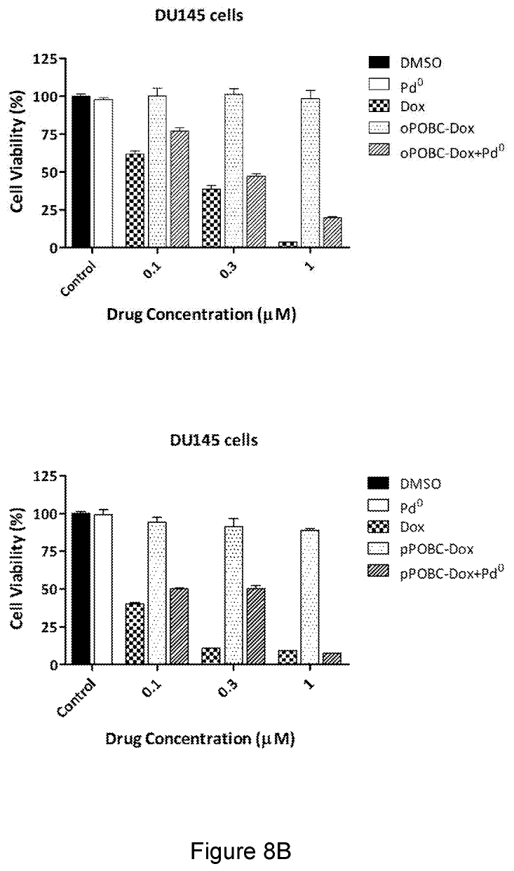

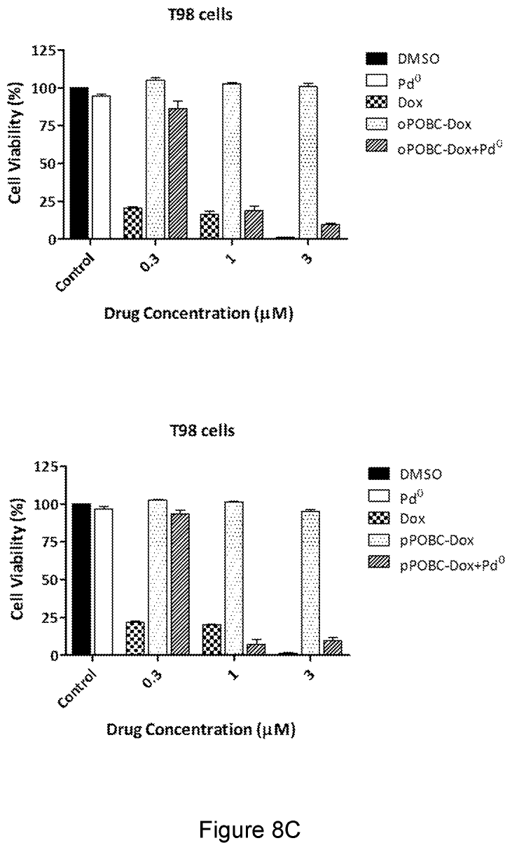

Following 5 days treatment, cells were incubated with PrestoBlue™ Cell Viability Reagent (Life Technologies) for 90 min. Fluorescence intensity values were related to the untreated cells (100% cell viability). Data are provided in FIG. 8 for cells incubated in tissue culture media containing 0.1% (v/v) DMSO and: Pd⁰-resins (1 mg/mL, negative control); 0.3-3 μM of Doxorubicin prodrug for A549 and DU145 cell lines, 0.1-1 μM of Doxorubicin prodrug for T98 cell line (negative control); 0.3-3 μM of Doxorubicin for A549 and DU145 cell lines, 0.1-1 μM of Doxorubicin for T98 cell line (positive control); and Pd⁰-resin (1 mg/mL)+Doxorubicin prodrug (BOOM reaction assay). Cells incubated in 0.1% (v/v) DMSO in media were used as untreated cell control.

Carbamates—Gemcitabine (“Class III”)

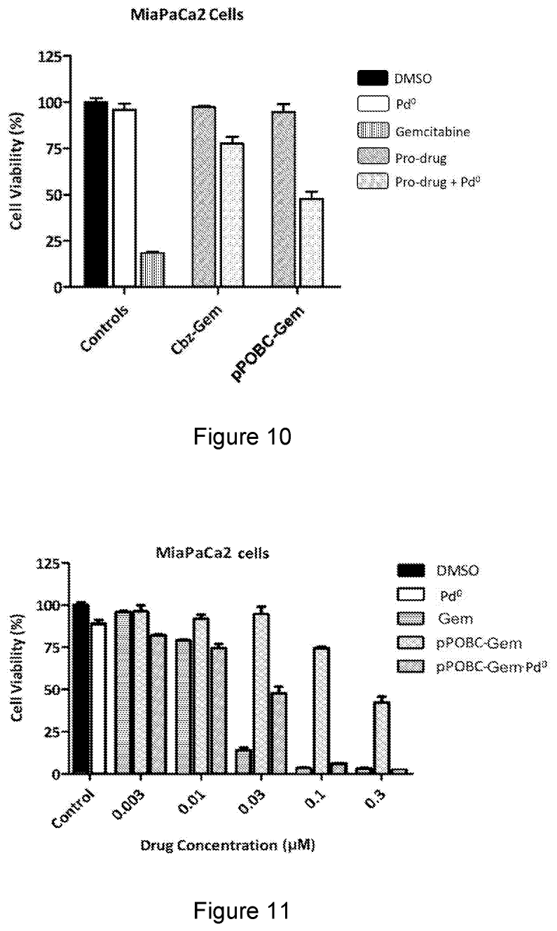

Cell viabilities for each prodrug provided as a combination with palladium is presented in FIG. 10 in the right of each set of two bars. Data for prodrug in the absence of palladium catalyst is also provided (the left bar of each set of two bars) for comparison along with negative controls (from left to right: DMSO, Pd⁰ and Gemcitabine). Cells were incubated in tissue culture media containing 0.1% (v/v) DMSO and: Pd⁰-resins (1 mg/mL, negative control); 0.03 μM of each prodrug (negative control); and Pd⁰-resins (1 mg/mL)+0.03 μM of each prodrug (BOOM reaction assay). Cells incubated in 0.1% (v/v) DMSO in media were used as untreated cell control.

Following 5 days treatment, cells were incubated with PrestoBlue™ Cell Viability Reagent (Life Technologies) for 90 min. Fluorescence intensity values were related to the untreated cells (100% cell viability). Data are provided in FIG. 11 for cells incubated in tissue culture media containing 0.1% (v/v) DMSO and: Pd⁰-resins (1 mg/mL, negative control); 0.003-0.3 μM of pPOBC-Gem (negative control); 0.003-0.3 μM of Gemcitabine (positive control); and Pd⁰-resin (1 mg/mL)+pPOBC-Gem (BOOM reaction assay). Cells incubated in 0.1% (v/v) DMSO in media were used as untreated cell control.

Prop-O-Drug—5-FU (“Class I”)

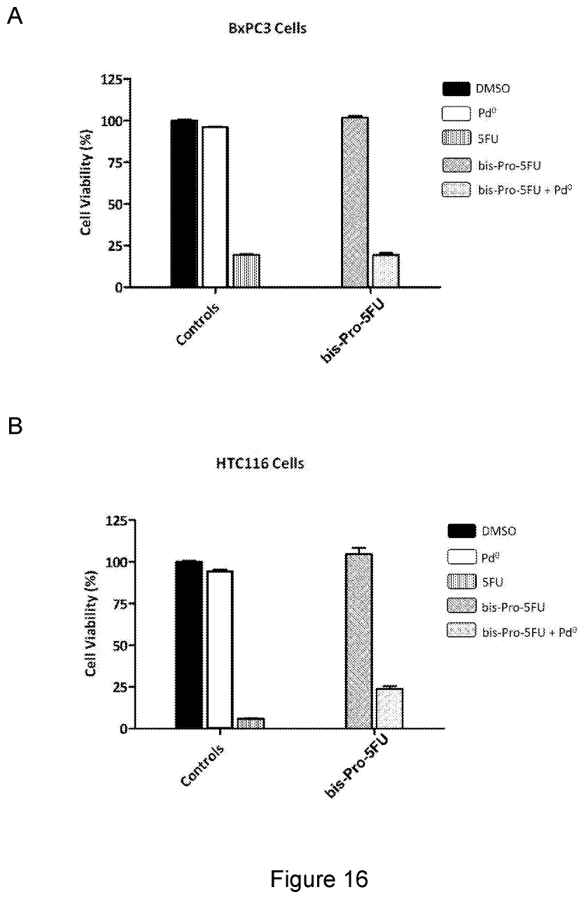

Cell viabilities for each prodrug provided as a combination with palladium is presented in FIG. 16 in the right of each set of two bars. Data for prodrug in the absence of palladium catalyst is also provided (the left bar of each set of two bars) for comparison along with negative controls (from left to right: DMSO, Pd⁰ and 5FU). Cells were incubated in tissue culture media containing 0.1% (v/v) DMSO and: Pd⁰-resins (1 mg/mL, negative control); 3 μM (for BxPC3 cells) or 30 μM (for HCT116 cells) of prodrug (negative control); and Pd⁰-resins (1 mg/mL)+3 μM (BxPC3) or 30 μM (HCT116) of each prodrug (BOOM reaction assay). Cells incubated in 0.1% (v/v) DMSO in media were used as untreated cell control.

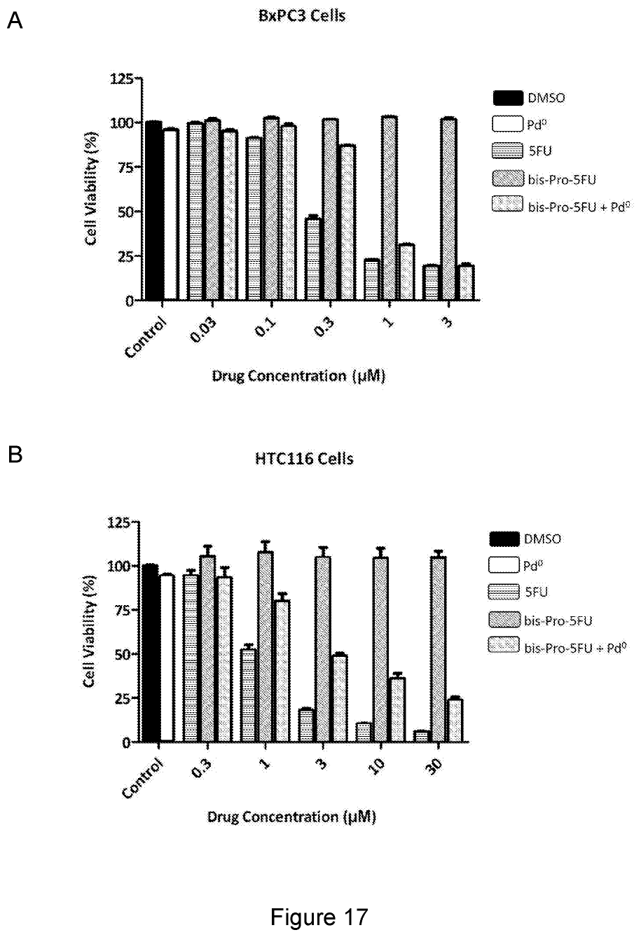

Following 5 days treatment, cells were incubated with PrestoBlue™ Cell Viability Reagent (Life Technologies) for 90 min. Fluorescence intensity values were related to the untreated cells (100% cell viability). Data are provided in FIG. 17 for cells incubated in tissue culture media containing 0.1% (v/v) DMSO and: Pd⁰-resins (1 mg/mL, negative control); 0.03-3 μM of bis-Pro-5FU for BxPC3, 0.3-30 μM of bis-Pro-5FU for HCT116 (negative control); 0.03-3 μM of 5FU for BxPC3, 0.3-30 μM of 5FU for HCT116 (positive control); and Pd⁰-resin (1 mg/mL)+bis-Pro-5FU (BOOM reaction assay). Cells incubated in 0.1% (v/v) DMSO in media were used as untreated cell control.

Hydroxamic Acids—Panobinostat (“Class II”)

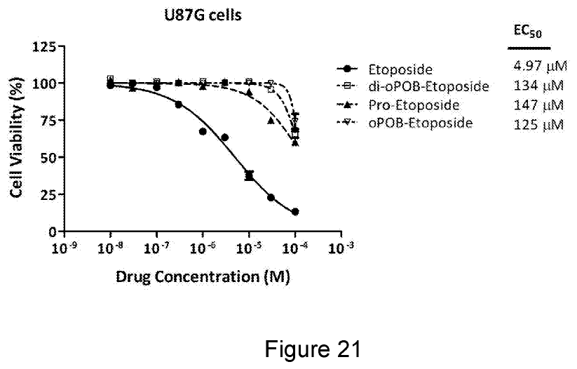

Cell viabilities for each prodrug provided as a combination with palladium is presented in FIG. 21 in the right of each set of two bars. Data for prodrug in the absence of palladium catalyst is also provided (the left bar of each set of two bars) for comparison along with negative controls (from left to right: DMSO, Pd⁰ and panobinostat). Cells were incubated in tissue culture media containing 0.1% (v/v) DMSO and: Pd⁰-resins (1 mg/mL, negative control); 1 μM of each prodrug (negative control); and Pd⁰-resins (1 mg/mL)+1 μM of each prodrug (BOOM reaction assay). Cells incubated in 0.1% (v/v) DMSO in media were used as untreated cell control.

The invention is described in more detail by way of example only with reference to the following Examples.

General Methods

Materials Synthesis and Characterization

Chemicals and solvents were obtained from Fisher Scientific, Sigma-Aldrich or VWR International Ltd. Resins were purchased from Rapp Polymere GmbH and Merck Millipore. NMR spectra were recorded at ambient temperature on a 500 MHz Bruker Avance III spectrometer. Chemical shifts are reported in parts per million (ppm) relative to the solvent peak. High Resolution Mass Spectrometry was measured in a Bruker MicroTOF II. R_{f} values were determined on Merck TLC Silica gel 60 F254 plates under a 254 nm UV source 0.1% ninhydrin solution in acetone for TLC staining. Purification of compounds was carried out by flash column chromatography using commercially available silica gel (220-440 mesh, Sigma-Aldrich).

Synthetic Procedure of Au Resins

TentaGel® HL NH₂ resins (250 mg, 0.4-0.6 mmol/g, particle size 110 μm or 75 μm) were added into a 25 mL Biotage microwave vial and suspended in THF (2.5 mL). A solution of gold(III) chloride hydrate (120 mg, 0.35 mmol) in distilled water (500 μL) was basified with a 1 M NaOH aqueous solution (11 μL). This freshly prepared solution was immediately added to the suspended resins and heated to 60° C. under stirring for 10 min. The mixture was then stirred at r.t. for additional 2 h. Subsequently, the solvents were filtered and the resins washed with DMF (3×10 mL), DCM (3×10 mL) and methanol (3×10 mL). Tetrakis(hydroxymethyl)phosphonium chloride (THPC) solution 80% in water (93 μL) was diluted in distilled water (6 mL) and a 1 M NaOH aqueous solution (11 μL) added. This solution was added to the gold(III)-treated resins and bubbled with a N₂ flow at r.t. for 25 min. The solvents were then filtered off and the resins washed with methanol (3×10 mL) and DCM (3×10 mL). Resins were then added to a solution of Fmoc-Glu(OH)-OH (64 mg, 0.17 mmol), oxyma (50 mg, 0.35 mmol), N,N′-diisopropylcarbodiimide (54 μL, 0.35 mmol) and DCM/DMF (3:1, 8 mL) and stirred for 2 h at r.t. The solvents were filtered off and the resins washed with DMF (1×10 mL), DCM (3×10 mL) and methanol (3×10 mL). Finally, resins were dispersed and shaken in a solution of acetic anhydride (60 μL) in DCM (10 mL) for 1h at r.t. The solvents were filtered and the resins were washed with DCM (3×10 mL) and methanol (3×10 mL). Resins were treated on the wheel overnight with methanol. The solvents were then filtered and resins were dried in an oven at 40° C. for 1 day.

Synthetic Procedure of Au—Pd Resins.

TentaGel® HL NH₂ resins (250 mg, 0.4-0.6 mmol/g, particle size 110 μm or 75 μm) were added into a 25 mL Biotage microwave vial and suspended in toluene (2.5 mL). Palladium(II) acetate (32.8 mg, 0.17 mmol) and gold(III) acetate (62.5 mg, 0.17 mmol) were added into the vial in one portion. The dispersion was immediately heated to 80° C. under stirring for 10 min. The mixture was then stirred at r.t. for additional 2 h. Subsequently, the solvents were filtered and the resins washed with DMF (3×10 mL), DCM (3×10 mL) and methanol (3×10 mL). A solution of 10% hydrazine monohydrate in methanol was added to the resin (5 mL). The suspension was then stirred at r.t. for 25 min. The solvents were then filtered off and the resins washed with methanol (3×10 mL) and DCM (3×10 mL). Resins were then added to a solution of Fmoc-Glu(OH)-OH (64 mg, 0.17 mmol), oxyma (50 mg, 0.35 mmol), N,N′-diisopropylcarbodiimide (54 μL, 0.35 mmol) and DCM/DMF (3:1, 8 mL) and stirred for 2 h at r.t. The solvents were filtered off and the resins washed with DMF (1×10 mL), DCM (3×10 mL) and methanol (3×10 mL). Finally, resins were dispersed and shaken in a solution of acetic anhydride (60 μL) in DCM (10 mL) for 1 h at r.t. The solvents were filtered and the resins were washed with DCM (3×10 mL) and methanol (3×10 mL). Resins were treated on the wheel overnight with methanol. The solvents were then filtered and resins were dried in an oven at 40° C. for 1 day.

### Synthesis of Vorinostat Prodrugs

### Synthesis of 4-propargyloxy-benzyl bromide

4-propargyloxy-benzyl alcohol was synthesised by propargylation of 4-hydroxylbenzyl alcohol using K₂CO₃ as base (Luo, J. et al. Chem Commun 21, 2136 (2007)). Alcohol was then converted in the corresponding halogenated intermediate using CBr₄/PPh₃ as previously described (Binauld, S. et al. Chem Commun 35, 4138 (2008)).

### General Method for the Synthesis of Vorinostat Prodrugs

Vorinostat (60 mg, 0.23 mmol) and 1,8-diazabicyclo[5.4.0]undec-7-ene (0.27 mmol) were dissolved in dry acetonitrile (1 mL) under N₂ atmosphere and cooled to 4° C. Either 4-propargyloxy-benzyl or benzyl bromide (0.23 mmol) were dissolved in dry acetonitrile (0.5 mL). The solution was added dropwise to the mixture and the resulting mixture stirred at room temperature for 24 h. Solvent was then removed under reduced pressure and the crude purified via flash chromatography eluting with AcOEt:Hexane (2:1).

### Propargyloxybenzyl-Vorinostat (POB-Vor)

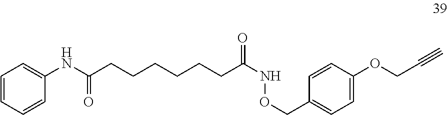 [SMILES: C#CCOc1ccc(CONC(=O)CCCCCCC(=O)Nc2ccccc2)cc1]

The synthetic method described above using 4-propargyloxy-benzyl bromide gave a white solid (30 mg, 32% yield). ¹H NMR (500 MHz, DMSO) δ 10.86 (s, 1H), 9.82 (s, 1H), 7.58 (d, J=7.7 Hz, 2H), 7.32 (d, J=8.6 Hz, 2H), 7.27 (m, 2H), 7.01 (t, J=7.4 Hz, 1H), 6.98 (d, J=8.6 Hz, 2H), 4.79 (d, J=2.4 Hz, 2H), 4.70 (s, 2H), 3.55 (t, J=2.4 Hz, 1H), 2.28 (t, J=7.4 Hz, 2H), 1.94 (t, J=7.3 Hz, 2H), 1.57 (m, 2H), 1.49 (m, 2H), 1.27 (m, 4H). ¹³C NMR (126 MHz, DMSO) δ 171.18 (C), 169.26 (C), 157.19 (C), 139.33 (C), 130.43 (CH), 128.84 (C), 128.60 (CH), 122.88 (CH), 119.01 (CH), 114.56 (CH), 79.19 (C), 78.20 (C), 76.27 (CH₂), 55.36 (CH₂), 36.34 (CH₂), 32.21 (CH₂), 28.33 (d, J=10.4 Hz, CH₂), 24.90 (d, J=17.4 Hz, CH₂). HRMS (ESI) m/z [M+Na]⁺ calculated for C₂₄H₂₈O₄N₂Na, 431.1941; found, 431.1949.

### Benzyl-Vorinostat (Benzyl-Vor)

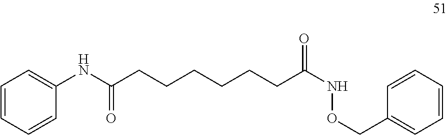 [SMILES: O=C(CCCCCCC(=O)Nc1ccccc1)NOCc1ccccc1]

The synthetic method described above using benzyl bromide gave a white solid (21.5 mg, 27% yield). ¹H NMR (500 MHz, DMSO) δ 10.91 (s, 1H), 9.82 (s, 1H), 7.58 (d, J=7.6 Hz, 2H), 7.38 (d, J=4.4 Hz, 4H), 7.34 (m, 1H), 7.27 (m, 2H), 7.01 (t, J=7.4 Hz, 1H), 4.77 (s, 2H), 2.28 (t, J=7.4 Hz, 2H), 1.94 (t, J=7.3 Hz, 2H), 1.57 (m, 2H), 1.49 (m, 2H), 1.26 (m, 4H). ¹³C NMR (126 MHz, DMSO) δ 171.17 (C), 169.32 (C), 139.33 (C), 136.09 (C), 128.66 (d, J=15.7 Hz, CH), 128.20 (d, J=11.8 Hz, CH), 122.87 (CH), 119.00 (CH), 76.71 (CH₂), 36.33 (CH₂), 32.19 (CH₂), 28.31 (d, J=12.5 Hz, CH₂), 24.89 (d, J=18.5 Hz, CH₂). HRMS (ESI) m/z [M+Na]⁺ calculated for C₂₁H₂₆O₃N₂Na, 377.1835; found, 377.1836.

### Synthesis of Doxorubicin Prodrugs

### Synthesis of Propargyloxy-benzyl Alcohols

2-propargyloxy-benzyl alcohol and 4-propargyloxy-benzyl alcohol were synthesised according to literature procedure (Luo, J. et al. Chem Commun 21, 2136 (2007)).

### Synthesis of 4-nitrophenyl-carbonate Promoieties

A solution of 4-nitrophenylchloroformate (0.48 g, 2.4 mmol) in dry DCM (8 mL) was added drop wise to a solution of either 2-propargyloxy-benzyl alcohol, 4-propargyloxy-benzyl alcohol or benzyl alcohol (2.2 mmol) and pyridine (0.19 mL, 2.4 mmol) in DCM (8 mL) at 0° C. under nitrogen in the dark. The mixture was stirred from 0° C. to room temperature overnight with TLC monitoring at t=0 hr, 0.5 hr, 2 hr and 20 hr, indicating full consumption of the alcohol. After concentrating in-vacuo, the crude residue was re-dissolved in ethyl acetate (70 mL), washed with water (2×50 mL) and brine (2×50 mL), dried over MgSO₄ and re-concentrated in-vacuo, and the crude was purified via flash chromatography (50% DCM in hexane).

### 4-nitrophenyl-2-propargyloxy-benzyl Carbonate

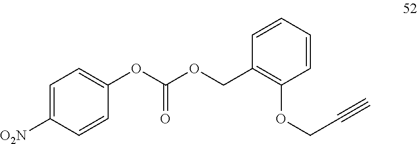 [SMILES: C#CCOc1ccccc1COC(=O)Oc1ccc([N+](=O)[O-])cc1]

The synthetic method described above using 2-propargyloxy-benzyl alcohol gave an off white oil that solidified to a white solid when below 20° C. (0.52 g, 1.59 mmol, 72% yield); R_{f} 0.19 (50% DCM in hexane). ¹H NMR (500 MHz, CDCl₃) δ 8.28-8.23 (m, 2H), 7.44-7.36 (m, 4H), 7.08-7.01 (m, 1H), 5.39 (s, 1H), 4.78 (d, J=2.4 Hz, 1H), 2.53 (t, J=2.4 Hz, 1H). ¹³C NMR (126 MHz, CDCl₃) δ 155.86 (s), 155.75 (s), 152.53 (s), 145.42 (s), 130.57 (d, J=12.8 Hz), 125.35 (s), 123.35 (s), 121.88 (s), 121.65 (s), 112.38 (s), 78.39 (s), 77.36 (s), 75.96 (s), 66.58 (s), 56.26 (s). HRMS (m/z): [M+Na]⁺ calcd for C₁₇H₁₃O₆N₁ [M+Na]⁺: 350.0635, found: 350.0590

### 4-nitrophenyl-4-propargyloxy-benzyl Carbonate

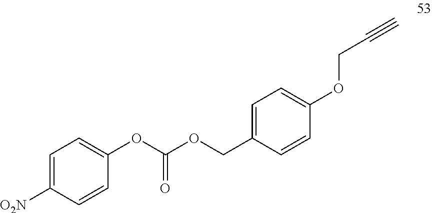 [SMILES: C#CCOc1ccc(COC(=O)Oc2ccc([N+](=O)[O-])cc2)cc1]

The synthetic method described above using 4-propargyloxy-benzyl alcohol gave an off white oil that solidified to a white solid when below 20° C. (0.54 g, 1.65 mmol, 75% yield); R_{f} 0.20 (50% DCM in hexane). ¹H NMR (500 MHz, CDCl₃) δ 8.28-8.24 (m, 2H), 7.42-7.38 (m, 2H), 7.38-7.35 (m, 2H), 7.03-6.98 (m, 2H), 5.24 (s, 2H), 4.71 (d, J=2.4 Hz, 2H), 2.53 (t, J=2.4 Hz, 1H). ¹³C NMR (126 MHz, CDCl₃) δ 158.30 (s), 155.68 (s), 152.57 (s), 145.51 (s), 130.72 (s), 127.39 (s), 125.39 (s), 121.88 (s), 115.27 (s), 78.38 (s), 77.37 (s), 75.90 (s), 70.87 (s), 55.95 (s). HRMS (m/z): [M+Na]⁺ calcd for C₁₇H₁₃O₆N₁ [M+Na]⁺: 350.0635, found: 350.0646

### 4-nitrophenyl-benzyl Carbonate

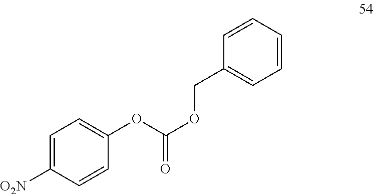 [SMILES: O=C(OCc1ccccc1)Oc1ccc([N+](=O)[O-])cc1]

The synthetic method described above using propargyl alcohol alcohol gave fluffy white crystals (0.41 g, 1.5 mmol, 68% yield); R_{f} 0.34 (50% DCM in hexane). ¹H NMR (500 MHz, CDCl₃) δ 8.30-8.26 (m, 2H), 7.48-7.36 (m, 7H), 5.30 (s, 2H). ¹³C NMR (126 MHz, CDCl₃) δ 155.63 (s), 152.59 (s), 145.49 (s), 134.27 (s), 129.23 (s), 128.95 (s), 128.85 (s), 125.46 (s), 121.93 (s), 77.37 (s), 71.15 (s). HRMS (m/z): [M+Na]⁺ calcd for C₁₄H₁₁O₅N₁ [M+Na]⁺: 296.0529, found: 296.0507.

### General Method for the Synthesis of Doxorubicin Prodrugs

A solution of the 4-nitrophenyl carbonate moiety (18 mg, 55.2 μmol, 1.5 equiv) in anhydrous DMF (2 mL) was flushed with nitrogen after stirring for 10 minutes, then syringed into a flask containing a solution of Doxorubicin-hydrochloride (20 mg, 36.8 μmol) and triethylamine (7.5 μL, 55.2 μmol) in anhydrous DMF under nitrogen at room temperature. The reaction was monitored by TLC (10% Methanol in DCM) at t=0 hr, 0.5 hr, 2 hr and 20 hr, monitoring for formation of product at R_{f} 0.88 (10% Methanol in DCM). After 20 hr, the reaction was diluted with water (50 mL) and extracted with ethyl acetate (4×50 mL). The combined organic extracts concentrated down to a volume of -100mL, then washed successively with saturated NaHCO₃ (2×50 mL), water (2×50 mL) and brine (2×50 mL), dried over MgSO4 and concentrated in-vacuo with the water bath kept below 40° C., and the crude was purified via flash chromatography (0→2% Methanol in DCM).

### 2-proparglyoxybenzylcarbamoyl Doxorubicin (oPOBC-Dox)

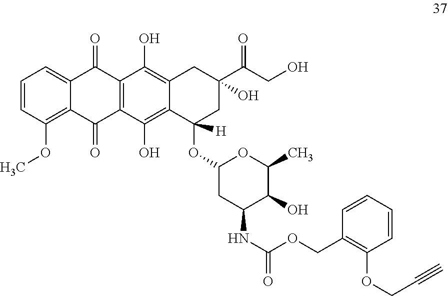

The synthetic method described above using 2-nitrophenyl-4-propargyloxy-benzyl carbonate gave a dark red clumpy powder (12.1 mg, 16.5 μmol, 45% yield); R_{f} 0.25 (2% Methanol in DCM). ¹H NMR (500 MHz, CDCl₃) δ 13.98 (s, 1H), 13.25 (s, 1H), 8.04 (dd, J=7.7, 1.0 Hz, 1H), 7.82-7.75 (m, J=8.4, 7.8 Hz, 1H), 7.39 (dd, J=8.5, 0.7 Hz, 1H), 7.33-7.27 (m, 1H), 7.01-6.93 (m, 2H), 5.51 (d, J=3.9 Hz, 1H), 5.33-5.27 (m, 1H), 5.15-5.08 (m, 3H), 4.76 (s, 2H), 4.70 (s, 2H), 4.55 (s, 1H), 4.14 (dd, J=12.3, 6.2 Hz, 1H), 4.08 (s, 3H), 3.92-3.82 (m, 1H), 3.68 (s, 1H), 3.28 (dd, J=18.8, 1.5 Hz, 1H), 3.03 (d, J=18.8 Hz, 2H), 2.48 (s, 1H), 2.34 (d, J=14.7 Hz, 1H), 2.17 (dd, J=14.7, 4.0 Hz, 1H), 1.88 (dd, J=13.5, 5.0 Hz, 1H), 1.77 (td, J=13.3, 4.2 Hz, 2H), 1.29 (d, J=6.6 Hz, 3H), 1.25 (s, 1H). ¹³C NMR (126 MHz, CDCl₃) δ 214.02 (s), 187.30 (s), 186.92 (s), 161.24 (s), 156.34 (s), 155.85 (s), 155.67 (d, J=26.0 Hz), 135.92 (s), 135.71 (s), 133.73 (s), 130.02 (s), 129.48 (s), 125.58 (s), 121.63 (s), 121.11 (s), 120.02 (s), 118.61 (s), 112.33 (s), 111.80 (s), 111.62 (s), 111.54-111.45 (m), 100.86 (s), 78.67 (s), 77.37 (s), 76.78 (s), 75.74 (s), 69.78 (s), 69.72 (s), 67.42 (s), 65.70 (s), 62.19 (s), 56.85 (s), 56.28 (s), 47.13 (s), 35.81 (s), 34.21 (s), 30.48-29.67 (m), 17.00 (s). HRMS (m/z): [M+Na]⁺ calcd for C₃₈H₃₇O₁₄N₁+²³Na₁ 754.2106 found 754.2130.

### 4-proparglyoxybenzylcarbamoyl Doxorubicin (pPOBC-Dox)

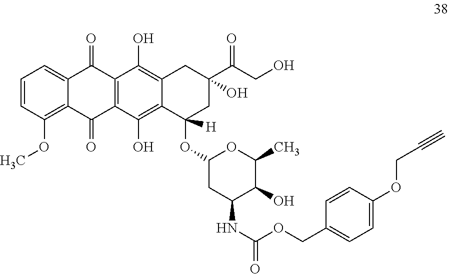

The synthetic method described above using 4-nitrophenyl-4-propargyloxy-benzyl carbonate gave a bright red clumpy powder (17.3 mg, 23.6 μmol, 64% yield); R_{f} 0.25 (2% Methanol in DCM). ¹H NMR (500 MHz, CDCl₃) δ 13.96 (s, 1H), 13.23 (s, 1H), 8.03 (dd, J=7.7, 1.0 Hz, 1H), 7.81-7.76 (m, J=8.3, 7.8 Hz, 1H), 7.39 (dd, J=8.5, 0.7 Hz, 1H), 7.24 (s, 1H), 6.92 (d, J=8.0 Hz, 2H), 5.49 (d, J=3.9 Hz, 1H), 5.28 (s, 1H), 5.10 (d, J=8.3 Hz, 1H), 4.97 (s, 2H), 4.76 (s, 2H), 4.66 (s, 2H), 4.53 (s, 1H), 4.17-4.10 (m, 1H), 4.08 (s, 3H), 3.91-3.80 (m, 1H), 3.66 (s, 1H), 3.27 (dd, J=18.8, 1.8 Hz, 1H), 3.05-2.97 (m, 2H), 2.49 (t, J=2.4 Hz, 1H), 2.33 (dt, J=14.5, 1.8 Hz, 1H), 2.17 (dd, J=14.7, 4.0 Hz, 1H), 1.93 (d, J=4.2 Hz, 1H), 1.87 (dd, J=13.5, 4.8 Hz, 1H), 1.76 (td, J=13.3, 4.2 Hz, 1H), 1.28 (d, J=6.6 Hz, 3H), 1.25 (s, 1H). ¹³C NMR (126 MHz, CDCl₃) δ 213.99 (s), 187.26 (s), 186.87 (s), 161.22 (s), 157.63 (s), 156.33 (s), 155.81 (s), 155.69 (s), 135.92 (s), 135.67 (s), 133.71 (d, J=4.9 Hz), 130.05 (s), 129.58 (s), 121.05 (s), 120.01 (s), 118.61 (s), 115.05 (s), 111.77 (s), 111.59 (s), 100.88 (s), 78.56 (s), 77.37 (s), 76.77 (s), 75.74 (s), 69.84 (s), 69.73 (s), 67.41 (s), 66.60 (s), 65.69 (s), 56.83 (s), 55.94 (s), 47.11 (s), 35.79 (s), 34.17 (s), 30.53-29.46 (m), 16.98 (s). HRMS (m/z): [M+Na]⁺ calcd for C₃₈H₃₇O₁₄N₁+²³Na₁ 754.2106 found 754.2130.

### Carboxybenzyl Doxorubicin (Cbz-Dox)

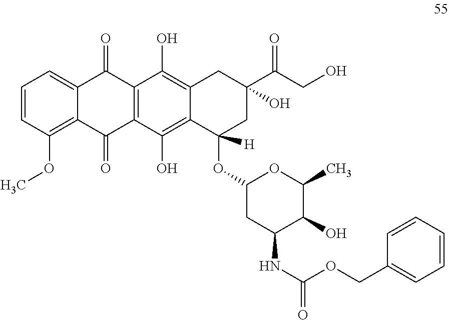

The synthetic method described above using 4-nitrophenyl-benzyl carbonate gave a dark red powder (17.3 mg, 25.5 μmol, 94% yield); R_{f} 0.25 (2% Methanol in DCM). ¹H NMR (500 MHz, CDCl₃) δ 13.96 (s, 1H), 13.23 (s, 1H), 8.03 (dd, J=7.7, 1.0 Hz, 1H), 7.78 (dd, J=8.3, 7.9 Hz, 1H), 7.39 (dd, J=8.6, 0.7 Hz, 1H), 7.34-7.26 (m, 5H), 5.50 (d, J=3.9 Hz, 1H), 5.28 (s, 1H), 5.14 (d, J=8.4 Hz, 1H), 5.03 (s, 2H), 4.82-4.70 (m, 2H), 4.54 (s, 1H), 4.14 (dd, J=13.6, 7.2 Hz, 1H), 4.08 (s, 3H), 3.92-3.82 (m, 1H), 3.67 (s, 1H), 3.27 (dd, J=18.8, 1.9 Hz, 1H), 3.00 (d, J=18.8 Hz, 2H), 2.33 (d, J=14.7 Hz, 1H), 2.19-2.14 (m, 1H), 1.88 (dd, J=13.5, 4.8 Hz, 1H), 1.77 (td, J=13.2, 4.1 Hz, 1H), 1.29 (d, J=6.6 Hz, 3H), 1.25 (s, 1H). ¹³C NMR (126 MHz, CDCl₃) δ 213.99 (s), 215.75-206.09 (m), 187.25 (s), 186.85 (s), 161.22 (s), 156.33 (s), 155.81 (s), 155.66 (s), 136.48 (s), 135.92 (s), 135.66 (s), 133.77-133.65 (m), 128.65 (s), 128.26 (s), 121.04 (s), 120.01 (s), 118.61 (s), 111.76 (s), 111.58 (s), 100.88 (s), 77.37 (s), 76.77 (s), 69.84 (s), 69.73 (s), 67.41 (s), 66.93 (s), 65.69 (s), 56.83 (s), 47.14 (s), 35.79 (s), 34.16 (s), 31.06 (s), 30.45-29.69 (m), 29.42 (s), 16.98 (s). HRMS (m/z): [M+Na]⁺ calcd for C₃₅H₃₅O₁₃N₁+²³Na₁ 700.2001 found 700.1988.

### Synthesis of Gemcitabine Prodrugs

### Synthesis of tert-Butyldimethylsilyl-Gemcitabine (TSB-Gem)

Silylated derivative was synthesized as previously described (Weiss, J. T. et al. J Med Chem 57, 5395 (2014)).

### Synthesis of carbamate Protected TBS-Gemcitabine

Dry pyridine (35.5 μL, 440.7 μmol, 2.7 equiv.) was added drop wise to a solution of TBS-Gemcitabine (60 mg, 162 μmol, 1 equiv.) and the 4-nitrophenyl carbonate moiety (106 mg, 324 μmol, 2 equiv.) in dry THF (2 mL) with rapid stirring, and the reaction monitored by TLC (5% Methanol in DCM) at t=1 hr, 6 hr and 24 hr, monitoring for formation of product at R_{f} 0.13 (10% Methanol in DCM). After 24 hr the mixture was concentrated in-vacuo, and the crude was purified via flash chromatography (0 4 5% Methanol in DCM).

### 2-propargyloxybenzylcarbamoyl-tert-Butyldimethylsilyl-Gemcitabine

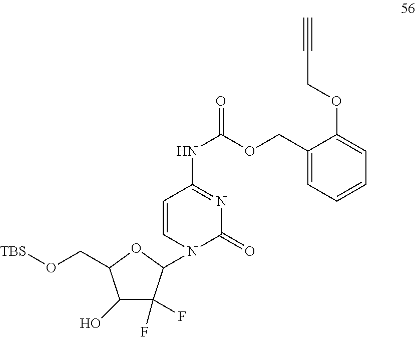

The synthetic method described above gave a white powder (6.4 mg, 11.3 μmol, 6.9% yield); R_{f} 0.28 (2.5% Methanol in DCM). ¹H NMR (500 MHz, MeOD) δ 8.26 (d, J=7.7 Hz, 1H), 7.43 (dd, J=7.5, 1.6 Hz, 1H), 7.39-7.32 (m, 2H), 7.16-7.12 (m, 1H), 7.03 (td, J=7.5, 1.0 Hz, 1H), 6.27 (t, J=6.8 Hz, 1H) 5.35-5.25 (m, 2H), 4.82 (d, J=2.4 Hz, 2H), 4.31 (td, J=12.5, 8.7 Hz, 1H), 4.12 (d, J=12.0 Hz), 4.03 (dt,J=8.7, 2.3 Hz, 1H), 3.95 (dd, J=12.1, 2.4 Hz, 1H), 2.96 (t, J=2.4 Hz, 1H), 1.00 (s, 9H), 0.19 (s, 6H). HRMS (m/z): [M+Si]⁺ calcd for C₂₆H₃₄O₇N₃F₂+Si²⁸ 566.21286 found 566.21470

### 4-propargyloxybenzylcarbamoyl-tert-Butyldimethylsilyl-Gemcitabine

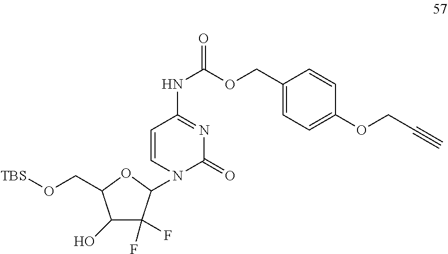

The synthetic method described above gave a white powder (8.5 mg, 15 μmol, 9% yield); R_{f} 0.30 (2.5% Methanol in DCM). ¹H NMR (500 MHz, MeOD) δ 8.26 (d, J=7.7 Hz, 1H), 7.38-7.35 (m, 2H), 7.31 (d, J=7.7 Hz, 1H), 7.00-6.97 (m, 2H), 6.24 (t, J=6.7 Hz, 1H), 5.16 (d, J=1.4 Hz, 2H), 4.72 (d, J=2.4 Hz, 2H), 4.28 (td, J=12.5, 8.7 Hz, 1H), 4.09 (d, J=12.0 Hz, 1H), 4.00 (dt, J=8.7, 2.2 Hz, 1H), 3.92 (dd, J=12.1, 2.3 Hz, 1H), 2.92 (t, J=2.4 Hz, 1H), 0.97 (s, 9H), 0.16 (s, 6H). ¹³C NMR (126 MHz, MeOD) δ 165.36 (s), 159.35 (s), 157.32 (s), 154.54 (s), 144.78 (s), 131.19 (s), 129.80 (s), 123.87 (t,J=258.8 Hz), 116.02 (s), 96.98 (s), 86.22 (t, J=32.5 Hz), 82.45 (s), 79.65 (s), 76.82 (s), 69.66 (d, J=22.9 Hz), 68.54 (s), 61.68 (s), 56.64 (s), 26.37 (s), 19.26 (s), 18.53 (s), −5.34 (s), −5.44 (s). HRMS (m/z): [M+Si]⁺ calcd for C₂₆H₃₄O₇N₃F₂+Si²⁸ 566.21286 found 566.21550.

### General Method for the Synthesis of Gemcitabine Prodrugs

The carbamate protected TBS-Gemcitabine (6.4 mg, 11.3 μmol—example for o-derivative; 8.5 mg, 15 μmol -example for p-derivative) was dissolved in dry THF (2 mL) and TBAF (30 μL, 101.8 μmol) was added. The solution was stirred rapidly for 24 hr, then concentrated in-vacuo and the crude was purified via flash chromatography (0→5% Methanol in DCM).

### 2-proparglyoxybenzylcarbamoyl Gemcitabine (oPOBC-Gem)

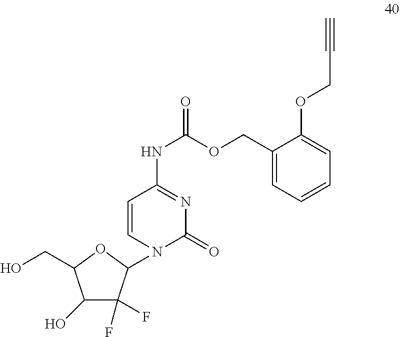 [SMILES: C#CCOc1ccccc1COC(=O)Nc1ccn(C2OC(CO)C(O)C2(F)F)c(=O)n1]

The synthetic method described above gave a white powder (5.1 mg, 11.3 μmol, 99% yield); R_{f} 0.40 (5% Methanol in DCM). ¹H NMR (601 MHz, MeOH) δ 8.31 (d, J=7.7 Hz, 1H), 7.40 (dd, J=7.5, 1.3 Hz, 1H), 7.38-7.29 (m, 2H), 7.11 (d, J=8.3 Hz, 1H), 7.00 (t, J=7.5 Hz, 1H), 6.33-6.18 (m, 1H), 5.28 (s, 2H), 4.80 (d, J=2.4 Hz, 2H), 4.36-4.24 (m, J=12.1, 8.8 Hz, 1H), 4.03-3.92 (m, 2H), 3.87-3.77 (m, 1H), 2.94 (t, J=2.4 Hz, 1H). ¹³C NMR (151 MHz, MeOH) δ 165.64 (s), 157.64 (s), 157.30 (s), 155.33 (s), 145.75 (s), 131.17 (s), 131.07 (s), 125.80 (s), 124.46 (s), 124.07 (s), 122.51 (s), 113.64 (s), 97.23 (s), 83.01-82.91 (m), 79.77 (s), 77.12 (s), 70.86-69.91 (m, J=24.2, 21.6 Hz), 64.37 (s), 60.43 (s), 57.21 (s). HRMS (m/z): [M+Na]⁺ calcd for C₂₀H₁₉O₇N₃F₂+Na²³ 474.10833 found 474.11050.

### 4-proparglyoxybenzylcarbamoyl Gemcitabine (pPOBC-Gem)

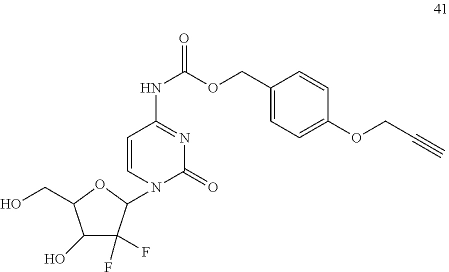 [SMILES: C#CCOc1ccc(COC(=O)Nc2ccn(C3OC(CO)C(O)C3(F)F)c(=O)n2)cc1]

The synthetic method described above gave a white powder (6.7 mg, 15 μmol, 99% yield); R_{f} 0.42 (5% Methanol in DCM). ¹H NMR (601 MHz, MeOD) δ 8.30 (d, J=7.7, 1H), 7.39-7.36 (m, 2H), 7.35 (d, J=7.6, 1H), 7.01-6.97 (m, 2H), 6.29-6.19 (m, 1H), 5.17 (s, 2H), 4.73 (d, J=2.4, 2H), 4.30 (td, J=12.1, 8.7, 1H), 4.02-3.91 (m, 2H), 3.81 (dd, J=12.7, 3.0, 1H), 2.93 (t, J=2.4, 1H). ¹³C NMR (151 MHz, MeOD) δ 165.61 (s), 159.52 (s), 157.62 (s), 154.66 (s), 145.78 (s), 131.34 (s), 130.04 (s), 125.78 (s), 124.07 (s), 116.17 (s), 97.18 (s), 83.11-82.88 (m), 79.81 (s), 76.95 (s), 70.89-70.00 (m), 68.64 (s), 60.44 (s), 56.79 (s). HRMS (m/z): [M+Na]⁺ calcd for C₂₀H₁₉O₇N₃F₂+Na²³ 474.10833 found 474.10830.

### Synthesis of Histamine Derivatives

### General Method for the Synthesis of Carbomate-Protected Histamine Derivatives

2-(4-Imidazolyl)ethylamine dihydrochloride (25 mg, 0.14 mmol) was dissolved in dry DMF (1 mL) with triethylamine (28 μL, 0.21 mmol) under N₂ atmosphere. O- or p-(2-propynyloxy)phenyl)methyl 4-nitrophenyl carbonate (60 mg, 0.21 mmol) was added dropwise to the mixture. The mixture was stirred overnight at room temperature (r.t.). The solvents were then removed in vacuo, the crude redissolved with CH₂Cl₂ (5 mL), and washed with H₂O (5 mL). The aqueous layer was washed with CH₂Cl₂ (3×5 mL) and the combined organic layers dried over MgSO₄, filtered, and concentrated under reduce pressure. The crude was purified by column chromatography using 20% MeOH in DCM.

### 2-proparglyoxybenzylcarbamoyl Histamine (oPOBC-Hist)

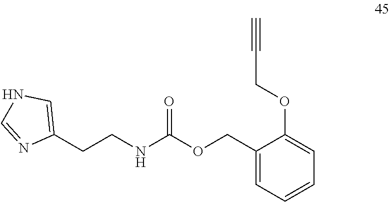 [SMILES: C#CCOc1ccccc1COC(=O)NCCc1c[nH]cn1]

The synthetic method described above gave a white solid (15 mg, 37% yield); Rf 0.39 (10% MeOH in DCM). ¹H NMR (500 MHz, MeOD) δ 8.74 (s, 1H), 7.30 (m, 3H), 6.98 (d, J=8.5, 2H), 5.00 (s, 2H), 4.75 (d, J=2.0, 2H), 3.45 (t, J=6.6, 2H), 2.98 (s, 1H), 2.93 (t, J=6.6, 2H). ¹³C NMR (126 MHz, MeOD) δ 157.58-157.51 (d, C═O), 133.38 (CH), 131.63 (C)₂, 129.75 (C), 129.24 (CH)₂, 116.25 (CH), 114.55 (CH)₂, 78.39 (CH), 75.55 (C), 65.84 (CH₂), 55.32 (CH₂), 39.34-39.22 (d, CH₂), 24.96 (CH₂). HRMS (ESI) m/z [M+H]+ calcd for C₁₆H₁₈O₃N₃, 300.13427; found, 300.13760.

### 4-proparglyoxybenzylcarbamoyl Histamine (pPOBC-Hist)

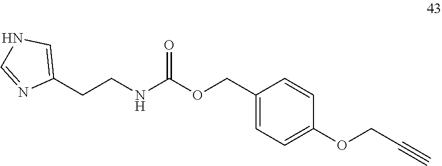 [SMILES: C#CCOc1ccc(COC(=O)NCCc2c[nH]cn2)cc1]

The synthetic method described above gave a white solid (35 mg, 85% yield); Rf 0.44 (10% MeOH in DCM). ¹H NMR (500 MHz, MeOD) δ 7.70 (s, 1H), 7.31 (dd, J=11.2, 7.7, 2H), 7.09 (d, J=8.1, 1H), 6.99 (t, J=7.4, 1H), 6.91 (s, 1H), 5.13 (s, 2H), 4.78 (d, J=2.2, 2H), 3.39 (t, J=7.1, 2H), 2.96 (t, J=2.3, 1H), 2.81 (t, J=7.0, 2H). ¹³C NMR (126 MHz, MeOD) δ 157.55 (C═O), 155.31 (C), 128.73-128.55 (d, CH)₂, 125.66 (C)₂, 120.91 (CH)₂, 111.98 (CH)₂, 78.36 (CH), 75.49 (C), 61.35 (CH₂), 55.63 (CH₂), 40.31 (CH₂), 26.76 (CH₂). HRMS (ESI) m/z [M+H]+ calcd for C₁₆H₁₈O₃N₃, 300.13427; found, 300.13830.

### Synthesis of 5FU Prodrug

### General Method for Synthesis of 5FU Prodrugs

Sodium hydride (60% in mineral oil), (120 mg, 3 mmol, 5 equiv.) was added to THF (10 mL) at 4° C. and stirred rapidly for 30 minutes. Propargyl alcohol (97 μL, 1.8 mmol 3 equiv.) was mixed in THF (5 mL) and added dropwise to the stirring mixture. An evolution of gas was observed with a slight exotherm. After stirring for an additional 10 minutes, the flask was sealed and flushed with nitrogen. A gas-tight syringe containing 5-fluoro-2,4-dichloropyrimidine (100 mg, 0.6 mmol, 1 equiv.) mixed in dry THF (5 mL) was added dropwise at 4° C. with rapid stirring, and allowed to warm to room temperature. The reaction mixture was monitored at t=1 hr, t=3 hr and t=24 hr, then diluted in DCM (50 mL), and the organic layer was washed twice with water (50 mL) and acidified with acetic acid. The organic layers were then concentrated in-vacuo and the crude was purified via flash chromatography (12.5% Ethyl Acetate in Hexane).

### 2,4-bispropargyl-5-fluorouracil (bis-Pro-5FU)

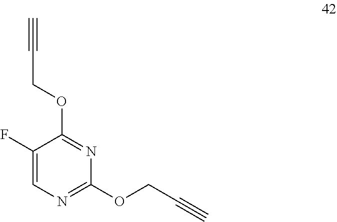

The synthetic method described above gave a white powder (45 mg, 0.22 mmol, 36.4% yield); R_{f} 0.40 (12.5% Ethyl Acetate in Hexane). ¹H NMR (500 MHz, CDCl3) δ 8.15 (d, J=2.3 Hz, 1H), 5.08 (d, J=2.4 Hz, 2H), 4.96 (d, J=2.4 Hz, 2H), 2.55 (t, J=2.4 Hz, 1H), 2.47 (t, J=2.4 Hz, 1H). ¹³C NMR (126 MHz, CDCl3) δ 158.61 (d, J=7.5 Hz), 144.20 (s), 144.10 (s), 143.94 (s), 142.17 (s), 78.27 (s), 76.15 (s), 75.00 (s), 55.73 (s), 55.09 (s). HRMS (m/z): [M+Na]⁺ calcd for C₁₀H₇O₂N₂F₁+Na²³ 229.03838 found 229.03760.

### Synthesis of Olaparib Prodrug

### General Method for the Synthesis of Olaparib Prodrug

Olaparib (AZD2281, MedChem Express LLC (MCE)) (10 mg, 0.03 mmol) was dissolved in dry DMF (1 mL) under N₂ atmosphere. The mixture was then cooled to 4° C. in an ice bath. Propargyl bromide solution 80 wt. % in toluene (8 μL, 0.05 mmol) was diluted in dry DMF (250 μL) and added to the mixture. Then, DBU (9 μL, 0.06 mmol) in dry DMF (250 μL) was added dropwise to the mixture. The mixture was stirred overnight and allowed to warm up to room temperature (r.t.). The solvents were then removed in vacuo, the crude redissolved with CH₂Cl₂ (5 mL), and washed with H₂O (5 mL). The aqueous layer was washed with CH₂Cl₂ (3×5 mL) and the combined organic layers dried over MgSO₄, filtered, and concentrated in vacuo. The crude was purified by Merck TLC Silica gel 60 F254 plates semipreparative using 5% MeOH in DCM.

### 4-[(3-[(4-cyclopropylcarbonyl)piperazin-4-yl]carbonyl)-4-fluorophenyl]methyl-phthalazin-1-oxypropargyl (Propargyl Olaparib, or Prop-Olap)

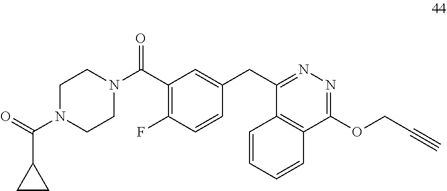 [SMILES: C#CCOc1nnc(Cc2ccc(F)c(C(=O)N3CCN(C(=O)C4CC4)CC3)c2)c2ccccc12]

The synthetic method described above gave a white solid (6 mg, 47% yield); Rf 0.39 (5% MeOH in DCM. ¹H NMR (500 MHz, CDCl3) δ 8.51 (m, 1H), 7.77 (m, 2H), 7.71 (m, 1H), 7.36 (m, 2H), 7.07 (t, J=8.9, 1H), 5.04 (d, J=2.5, 2H), 4.33 (s, 2H), 3.79 (m, 4H), 3.62 (m, 2H), 3.35 (m, 2H), 2.35 (t, J=2.4, 1H), 1.72 (m, 1H), 1.28 (t, J=7.1, 2H), 1.03 (dd, J=4.6, 2.9, 2H). ¹³C NMR (126 MHz, CDCl3) δ 172.35 (C═O), 171.15 (C═O), 158.73 (C), 158.02 (C), 156.05 (C), 145.01 (C), 134.44-134.41 (d, C), 133.33 (CH), 131.69-131.61 (d, CH)₂, 129.15 (C), 128.28 (C), 127.65 (CH)₂, 124.93 (CH), 116.31-116.14 (d, CH), 77.22 (CH), 72.43 (C), 60.39 (CH₂)₂, 53.49 (CH₂)₂, 40.73 (CH₂), 37.81 (CH₂), 11.04 (CH), 7.70 (CH₂)₂. HRMS (ESI) m/z [M+H]+ calcd for C₂₇H₂₆O₃N₄F₁, 473.19835; found, 473.19410.

### Synthesis of Panobinostat Prodrug

### Synthesis of N-[4-(propargyloxy)benzyloxy]phthalimide

N-[4-(propargyloxy)benzyloxy]phthalimide was synthesised by alkylation of N-hydroxyphthalimide with 4-propargyloxy-benzyl bromide using NaH as previously described for others O-alkylhydroxylamines (High, A et al. J Pharmacol Exp Ther. 1999, 288, 490-501).

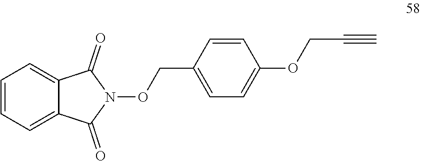 [SMILES: C#CCOc1ccc(CON2C(=O)c3ccccc3C2=O)cc1]

The synthetic method described above gave a pale-yellow solid (534 mg, 79% yield). ¹H NMR (500 MHz, DMSO) δ 7.86 (s, 4H), 7.45 (d, J=8.7 Hz, 2H), 7.00 (d, J=8.7 Hz, 2H), 5.10 (s, 2H), 4.81 (d, J=2.4 Hz, 2H), 3.56 (t, J=2.4 Hz, 1H). ¹³C NMR (126 MHz, DMSO) δ 163.12 (C×2), 157.78 (C), 134.77 (CH×2), 131.35 (CH×2), 128.51 (C×2), 126.92 (C), 123.22 (CH×2), 114.70 (CH×2), 79.06 (C), 78.78 (CH₂), 78.29 (CH), 55.41 (CH₂). HRMS (ESI) m/z [M+Na]⁺ calcd for C₁₈H₁₃O₄NNa, 330.0737; found, 330.0785.

### Synthesis of O-[4(propargyloxy)benzyl]hydroxylamine hydrochloride

O-[4(propargyloxy)benzyl]hydroxylamine was obtained by removal of the phthaloyl group by hydrazinolysis in diethyl ether and converted to the hydrochloride salt with an ethereal hydrochloric acid solution as reported by Bindman, N. A. et al. J Am Chem Soc. 2013, 135, 10362-10371.

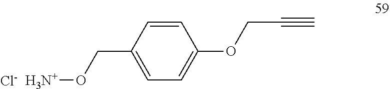

The synthetic method described above gave a white solid (190 mg, 77% yield). ¹H NMR (500 MHz, DMSO) δ 10.94 (s, 3H), 7.37 (d, J=8.7 Hz, 2H), 7.03 (d, J=8.7 Hz, 2H), 4.95 (s, 2H), 4.82 (d, J=2.4 Hz, 2H), 3.58 (t, J=2.4 Hz, 1H). ¹³C NMR (126 MHz, DMSO) δ 157.83 (C), 131.05 (CH×2), 126.29 (C), 114.92 (CH×2), 79.06 (C), 78.36 (CH), 75.39 (CH₂), 55.43 (CH₂). HRMS (ESI) m/z [M—Cl]⁺ calcd for C₁₀H₁₂O₂N, 178.0863; found, 178.0885.

### Synthesis of (E)-3-(4-{[2-(2-methyl-1H-indol-3-yl)ethylamino]methyl}phenyl)acrylic acid

(E)-Methyl 3-(4-{[2-(2-methyl-1H-indol-3-yl)ethylamino]methyl}phenyl)acrylate was synthesised according to literature procedure (Wang, H. et al. J Med Chem. 2011, 54, 4694-4720) and hydrolyzed to the corresponding carboxylic acid by treatment with NaOH.

### General Method for the Synthesis of Panobinostat Prodrug

(E)-3-(4-{[2-(2-methyl-1H-indol-3-yl)ethylamino]methyl}phenyl)acrylic acid (25 mg, 0.075 mmol) and O-[4(propargyloxy)benzyl]hydroxylamine hydrochloride (24 mg, 0.112 mmol) were added to a 25 mL round-bottom flask and partially dissolved in distilled water (1 mL). N-(3-Dimethylaminopropyl)-N′-ethylcarbodiimide hydrochloride (43 mg, 0.225 mmol) was then added to the mixture in one portion. The pH was monitored and adjusted to 4.5 with a NaOH and/or HCl aqueous solution (1 M). The reaction was stirred for 6 hours at room temperature and constant pH (4.5). Water was removed under reduced pressure, and crude suspended in acetonitrile and vacuum filtered. Solution was purified by semi-preparative TLC eluting with DCM:MeOH/7:3.

### Propargyloxybenzyl-Panobinostat (POB-Panob)

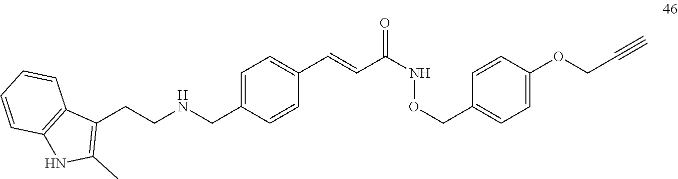 [SMILES: C#CCOc1ccc(CONC(=O)/C=C/c2ccc(CNCCc3c(C)[nH]c4ccccc34)cc2)cc1]

The synthetic method described above gave a yellow solid (9.3 mg, 25%). ¹H NMR (500 MHz, DMSO) δ 11.18 (s, 1H), 10.67 (s, 1H), 7.49 (m, 3H), 7.36 (m, 5H), 7.21 (d, J=7.9 Hz, 1H), 7.00 (d, J=8.7 Hz, 2H), 6.95 (m, 1H), 6.89 (m, 1H), 6.41 (d, J=15.8 Hz, 1H), 4.80 (s, 4H), 3.80 (s, 2H), 3.56 (s, 1H), 2.81 (m, 2H), 2.72 (m, 2H), 2.30 (s, 3H). LC-MS (m/z): 494.1749.

### Synthesis of SN-38 Prodrug

### Synthesis of Benzoic Acid, 2,6-bis(2-propyn-1-yloxy)-2-propyn-1-yl Ester

2,6-Dihydroxybenzoic acid (6.96 g, 45 mmol) and potassium carbonate (30.5 g, 220 mmol) were suspended in dry DMF (40 mL) and stirred for 30 mins at 0° C. Propargyl bromide (21 mL, 80% in toluene, 16.8 141 mmol) was added dropwise and the reaction was warmed to ambient temperature and stirred for three days. The reaction was diluted with water (300 mL) and extracted with diethyl ether (6×200 mL). The combined organic phases were washed with brine, dried over MgSO₄ and concentrated in vacuo to yield the title compound.

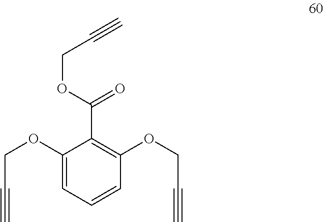 [SMILES: C#CCOC(=O)c1c(OCC#C)cccc1OCC#C]

The synthetic method described above gave a brown oil (5.37 g 20.1 mmol, 44%), used without further purification. ¹H NMR (400 MHz, CDCl₃) δ 7.32 (t, J=8.4 Hz, 1H), 6.76 (d, J=8.4 Hz, 2H), 4.91 (d, J=2.5 Hz, 2H), 4.71 (d, J=2.5 Hz, 4H), 2.51 (t, J=2.4 Hz, 2H), 2.50 (t, J=2.5 Hz, 1H); ¹³C NMR (126 MHz, CDCl₃) δ 165.1, 155.9, 131.3, 114.0, 106.8, 78.2, 77.7, 76.2, 75.2, 57.0, 52.9.

### Synthesis of (2,6-Bis(prop-2-yn-1-yloxy)phenyl)methanol

2,6-Bis(2-propyn-1-yloxy)-, 2-propyn-1-yl ester benzoic acid (5.37 g, 20 mmol) was dissolved in THF and cooled to 0° C. for the addition of LiAlH₄ (1 M in THF, 24 mL, 24 mmol) before warming to ambient temperature and stirring overnight. The reaction was quenched at 0° C. with 10% NaOH (40 mL), stirring for 30 mins. The aqueous phase was extracted with CH₂Cl₂ (3×70 mL) and the combined organic phases washed with brine (40 mL), dried over MgSO₄ and concentrated in vacuo. The crude alcohol was purified by flash column chromatography (30% AcOEt/hexane).

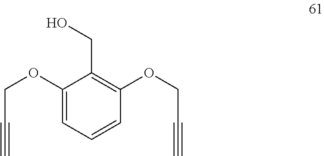 [SMILES: C#CCOc1cccc(OCC#C)c1CO]

The synthetic method described above gave a white solid (2.74 g, 12.6 mmol, 63%). ¹H NMR (500 MHz, CDCl₃) δ 7.24 (t, J=8.4 Hz, 1H), 6.72 (d, J=8.4 Hz, 2H), 4.81 (d, J=6.7 Hz, 2H), 4.74 (d, J=2.4 Hz, 4H), 2.51 (t, J=2.4 Hz, 2H), 2.37 (t, J=6.7 Hz, 1H). ¹³C NMR (126 MHz, CDCl₃) δ 162.5, 155.7, 129.1, 106.6, 78.5, 75.82, 56.6, 54.4.

### Synthesis of 2-(chloromethyl)-1,3-bis(prop-2-yn-1-yloxy)benzene

Cyanuric chloride (180 mg, 1.00 mmol) was stirred as a suspension in DMF (0.1 mL) for one hour. (2,6-Bis(prop-2-yn-1-yloxy)phenyl)methanol (194 mg, 0.90 mmol) in CH₂CL₂ (1 mL) was added and the reaction stirred at ambient temperature overnight. The reaction was diluted with CH₂CL₂ (25 mL) and washed with sat. bicarb. The aqueous phase was extracted with CH₂CL₂ (2×15 mL), dried over MgSO₄ and concentrated in vacuo. The crude product was further purified with column chromatography (40% AcOEt/hexane).

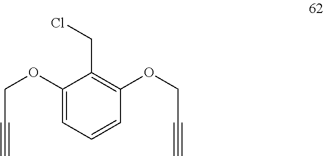 [SMILES: C#CCOc1cccc(OCC#C)c1CCl]

The synthetic method described above gave a white solid (161 mg, 0.69 mmol, 77%). ¹H NMR (500 MHz, CDCl₃) δ 7.28 (t, J=8.4 Hz, 1H) 6.72 (d, J=8.4 Hz, 2H), 4.78 (d, J=2.3 Hz, 4H), 4.78 (s, 2H), 2.51 (t, J=2.4 Hz, 2H); ¹³C NMR (126 MHz, CDCl₃) δ 156.7, 123.0, 115.9, 106.2, 78.4, 75.7, 56.0, 35.3.

### General Method for the Synthesis of SN-38 Prodrug

10-Hydroxy-7-ethylcamptothecin (SN-38, 40 mg, 0.10 mmol) and potassium carbonate (21 mg, 0.15 mmol) were dissolved in dry DMF (2 mL) and stirred for 10 minutes at 0° C. under nitrogen. 2,6-Bis(propargyloxy)benzyl chloride (28.1 mg, 0.21 mmol) in DMF (0.5 mL) was added and the reaction warmed to ambient temperature and stirred overnight. Solvent was evaporated in vacuo and the crude prodrug purified by semi-preparative TLC (3% MeOH/CH₂Cl₂).

### (S)-9-((2,6-bis(prop-2-yn-1-yloxy)benzyl)oxy)-4,11-diethyl-4-hydroxy-1,12-dihydro-14H-pyrano[3′,4′:6,7]indolizino[1,2-b]quinoline-3,14(4H)-dione (di-oPOB-SN-38)

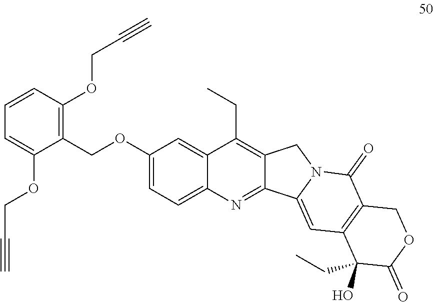 [SMILES: C#CCOc1cccc(OCC#C)c1COc1ccc2nc3c(c(CC)c2c1)Cn1c-3cc2c(c1=O)COC(=O)[C@]2(O)CC]

The synthetic method described above gave a white solid (2.7 mg, 3% yield). ¹H NMR (500 MHz, MeOD) δ 8.05 (d, J=9.3 Hz, 1H), 7.97 (s, 2H), 7.65 (s, 1H), 7.52 (dd, J=9.3, 2.6 Hz, 1H), 7.34 (t, J=8.5 Hz, 1H), 6.84 (d, J=8.5 Hz, 2H), 5.60 (d, J=16.2 Hz, 1H), 5.37 (s, 2H), 5.37 (d, J=16.2 Hz, 1H), 5.30 (s, 2H), 4.81 (d, J=2.4 Hz, 4H), 3.26 (q, J=7.6 Hz, 2H), 2.88 (t, J=2.4 Hz, 2H), 2.03-1.90 (m, J=7.0 Hz, 2H), 1.43 (t, J=7.6 Hz, 3H), 1.02 (t, J=7.4 Hz, 3H); ¹³C NMR (126 MHz, DMSO) δ173.0, 158.4, 157.6, 156.4, 150.5, 150.0, 146.8, 145.1, 144.5, 131.9, 130.9, 130.3, 128.8, 128.3, 123.1, 118.6, 113.4, 106.8, 105.1, 96.5, 79.6, 78.9, 72.9, 65.7, 56.7, 50.0, 30.7, 22.8, 14.0, 8.2.

### Synthesis of Etoposide Prodrugs

### General Method for the Synthesis of Etoposide Prodrugs

Etoposide (20 mg, 0.034 mmol) and potassium carbonate (7 mg, 0.051 mmol) were stirred in dry DMF (1 mL) at 0° C. for five mins. Alkyl halides (9 mg, 0.076 mmol—example for propargyl bromide; 9 mg, 0.050 mmol—example for 2-(Chloromethyl)-2-(prop-2-yn-1-yloxy)benzene; 12 mg, 0.051 mmol—example for (Chloromethyl)-1,3-bis(prop-2-yn-1-yloxy)benzene) in dry DMF (1 mL) was added and the reaction stirred at ambient temperature 6h for propargyl bromide; overnight for 2-(Chloromethyl)-2-(prop-2-yn-1-yloxy)benzene; and five days for (Chloromethyl)-1,3-bis(prop-2-yn-1-yloxy)benzene. Solvent was removed in vacuo and the crude compound purified by semi-preparative TLC (4% MeOH/CH₂Cl₂ for propargyl and 2-(methyl)-2-(prop-2-yn-1-yloxy)benzene; or 5% MeOH/CH₂Cl₂ for (methyl)-1,3-bis(prop-2-yn-1-yloxy)benzene).

### Propargyloxy Etoposide (Pro-Etoposide)

### (5R,5aR,8aR,9S)-9-(((2R,4aR,6R,7R,8R,8aS)-7,8-dihydroxy-2-methylhexahydropyrano[3,2-d][1,3]dioxin-6-yl)oxy)-5-(3,5-dimethoxy-4-(prop-2-yn-1-yloxy)phenyl)-5,8,8a,9-tetrahydrofuro[3′,4′:6,7]naphtho[2,3-d][1,3]dioxol-6(5aH)-one

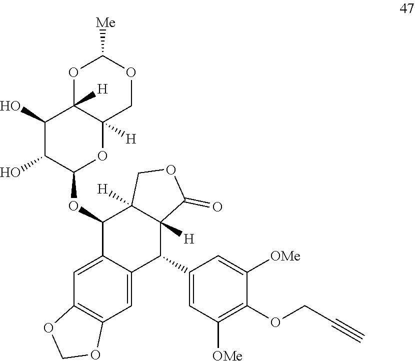

The synthetic method described above gave a white solid (14 mg, 0.022 mmol, 79%). ¹H NMR (500 MHz, MeOD) δ 6.99 (s, 1H), 6.52 (s, 1H), 6.32 (s, 2H), 5.98-5.95 (m, 2H), 5.03 (d, J=3.3 Hz, 1H), 4.76 (q, J=5.0 Hz, 1H), 4.65 (d, J=7.7 Hz, 1H), 4.61 (d, J=5.5 Hz, 1H), 4.57 (d, J=2.5 Hz, 2H), 4.43 (dd, J=10.7, 8.6 Hz, 1H), 4.29 (dd, J=8.7, 7.6 Hz, 1H), 4.17 (dd, J=10.3, 4.8 Hz, 1H), 3.71 (s, 6H), 3.58 (t, J=10.1 Hz, 1H), 3.54 (t, J=9.1 Hz, 1H), 3.48 (dd, J=14.2, 5.5 Hz, 1H), 3.30-3.23 (m, 2H), 2.99-2.91 (m, 1H), 2.81 (t, J=2.4 Hz, 1H), 1.32 (d, J=5.0 Hz, 3H); ¹³C NMR (126 MHz, MeOD) δ 177.6, 154.1, 150.1, 148.4, 138.1, 135.9, 133.9, 130.4, 111.2, 110.9, 109.4, 103.2, 102.9, 100.8, 81.8, 80.3, 76.14, 75.9, 74.5, 74.0, 69.7, 69.2, 67.6, 60.7, 56.6, 45.2, 42.2, 39.3, 20.6.

### Propargyloxybenzyl Etoposide (oPOB-Etoposide)

### (5R,5aR,8aR,9S)-9-(((2R,4aR,6R,7R,8R,8aS)-7,8-dihydroxy-2-methyl hexahydropyrano[3,2-d][1,3]dioxin-6-yl)oxy)-5-(3,5-dimethoxy-4-((2-(prop-2-yn-1-yloxy)benzyl)oxy)phenyl)-5,8,8a,9-tetrahydrofuro[3′,4′:6,7]naphtho[2,3-d][1,3]dioxol-6(5aH)-one

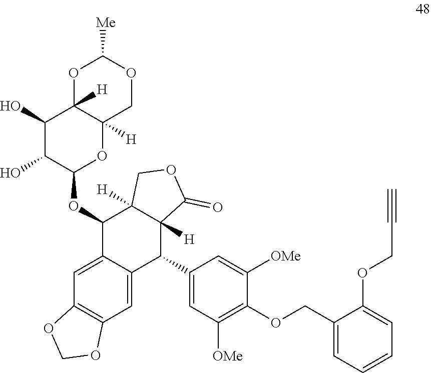

The synthetic method described above gave a white solid (4.7 mg, 0.0065 mmol, 19%). ¹H NMR (400 MHz, MeOD) δ 7.45 (dd, J=7.8, 1.2 Hz, 1H), 7.27 (td, J=8.3, 1.8 Hz, 1H), 7.06-7.03 (m, 1H), 7.01 (s, 1H), 6.96 (td, J=7.4, 0.9 Hz, 1H), 6.54 (s, 1H), 6.31 (s, 2H), 5.99 (dd, J=3.3, 1.0 Hz, 2H), 5.05 (d, J=3.4 Hz, 1H), 4.99 (s, 2H), 4.79 (q, J=5.0 Hz, 1H), 4.68 (d, J=2.5 Hz, 1H), 4.67 (m, 3H), 4.62 (d, J=5.6 Hz, 1H), 4.45 (dd, J=10.7, 8.6 Hz, 1H), 4.32 (t, J=8.2 Hz, 1H), 4.19 (dd, J=10.3, 4.8 Hz, 1H), 3.64-3.45 (m, 4H), 3.30-3.24 (m, 2H), 2.95 (t, J=2.4 Hz, 1H), 1.34 (d, J=5.0 Hz, 3H); ¹³C NMR (126 MHz, MeOD) δ 180.4, 155.7, 153.7, 147.8, 146.7, 137.8, 135.3, 132.1, 130.1, 128.8, 128.5, 126.6, 120.8, 112.1, 108.8, 107.3, 105.5, 101.8, 101.1, 99.3, 80.2, 78.1, 75.0, 74.5, 73.4 69.1, 68.7, 67.8, 66.1, 55.9, 55.3, 45.0, 43.5, 39.3, 29.4, 19.2.

Bis-propargyloxybenzyl Etoposide (di-oPOB-Etoposide)

(5R,5aR,8aR,9S)-5-(4-((2,6-bis(prop-2-yn-1-yloxy)benzyl)oxy)-3,5-dimethoxyphenyl)-9-(((2R,4aR,6R,7R,8R,8aS)-7,8-dihydroxy-2-methylhexahydropyrano[3,2-d][1,3]dioxin-6-yl)oxy)-5,8,8a,9-tetrahydrofuro[3′,4′:6,7]naphtho[2,3-d][1,3]dioxol-6(5aH)-one

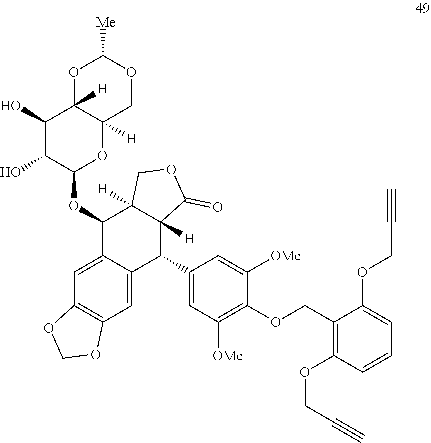

The synthetic method described above gave a white solid (7 mg, 0.009 mmol, 23%). ¹H NMR (500 MHz, MeOD) δ 7.23 (t, J=8.4 Hz, 1H), 7.12 (s, 1H), 6.73 (d, J=8.4 Hz, 2H), 6.45 (s, 3H), 5.93 (q, J=1.2 Hz, 2H), 5.18 (dd, J=11.3, 6.7 Hz, 2H), 4.80 (d, J=4.8 Hz, 1H), 4.74 (q, J=5.0 Hz, 1H), 4.58 (qd, J=15.7, 2.4 Hz, 4H), 4.43-4.33 (m, 3H), 4.26 (d, J=7.8 Hz, 1H), 4.09 (dd, J=10.4, 4.6 Hz, 1H), 3.66 (s, 6H), 3.66-3.54 (m, 2H), 3.54 (dd, J=10.7, 9.1 Hz, 1H), 3.46 (t, J=8.9 Hz, 1H), 3.29-3.16 (m, 4H), 2.94 (t, J=2.4 Hz, 2H), 1.30 (d, J=5.0 Hz, 3H); ¹³C NMR (126 MHz, MeOD) δ 181.9, 159.6, 155.4, 149.2, 148.0, 138.9, 136.4, 133.7, 131.0, 129.8, 116.7, 110.2, 108.7, 107.4, 106.7, 103.2, 102.5, 100.7, 81.6, 80.2, 76.8, 76.6, 75.9, 74.8, 70.1, 69.2, 67.6, 63.6, 57.7, 56.7, 46.4, 44.7, 40.7, 20.6.

Experimental Data

Cell-Free Palladium Mediated Deprotection of Prodrugs

To recreate a biocompatible scenario, prodrug-into-active agent conversion was carried out at 37° C. in an isotonic solution with a physiologic pH. POB-Vor, oPOBC-Hist and pPOBC-Hist (100 μM) were incubated in phosphate buffered saline (PBS, 1 ml) with 1 mg of Pd⁰ resin for 24 h at 37° C. (Thermomixer, shaker speed: 1,200 rpm). Reaction crudes were monitored at 0 h, 3h, 6 h by analytical HPLC using an UV-VIS detector (for POB-Vor) and analytical LCMS using a microTOF II detector (for oPOBC-Hist and pPOBC-Hist). HPLC method: eluent A: water and trifluoroacetic acid (0.4%); eluent B: acetonitrile; A/B=95:5 to 20:80 in 6 min, isocratic 1 min, 20:80 to 95:5 in 0.1 min, and isocratic 2 min with the UV detector at 254 nm. LCMS method: eluent A: water and formic acid (0.1%); eluent B: acetonitrile and formic acid (0.1%); A/B=95:5 isocratic 0.5 min, 95:5 to 0:100 in 4.5 min, isocratic 2 min, 0:100 to 95:5 in 0.5 min, and isocratic 2.5 min (flow=0.2 mL/min).

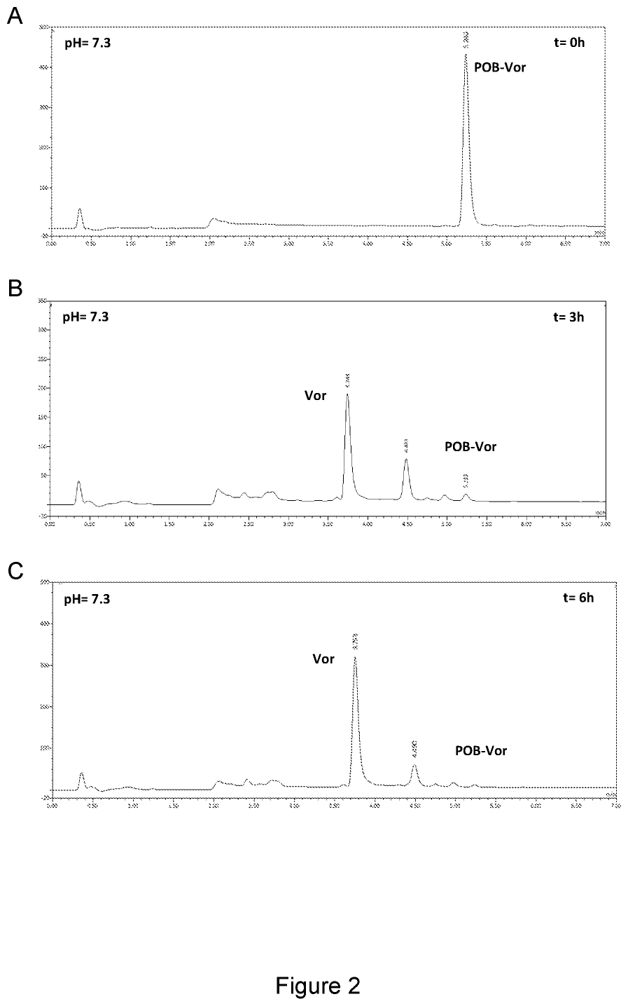

FIG. 2 shows the HPLC chromatograms for the Pd-catalysed deprotection of POB-Vor at times of 0 h, 3 h and 6 h. As seen in FIG. 2C, POB-Vor completely disappeared from the crude mixture after 6 h, with Vorinostat being the major reaction product.

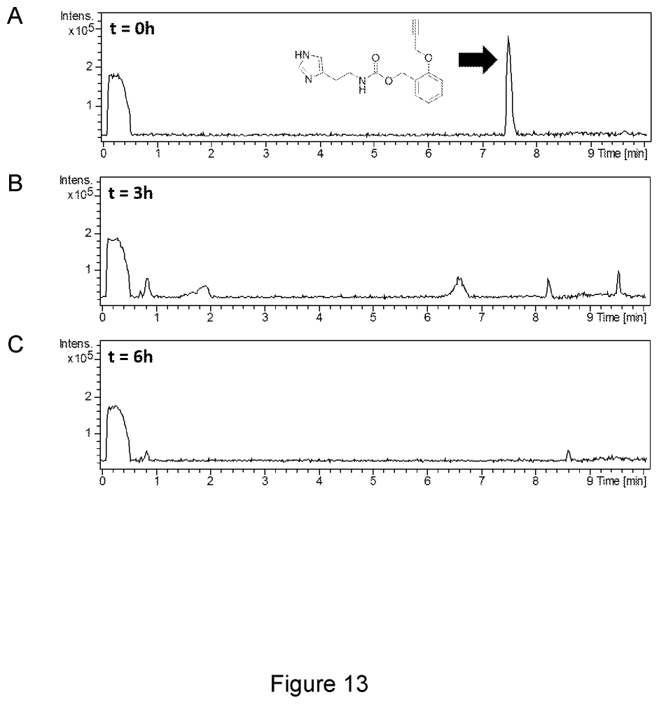

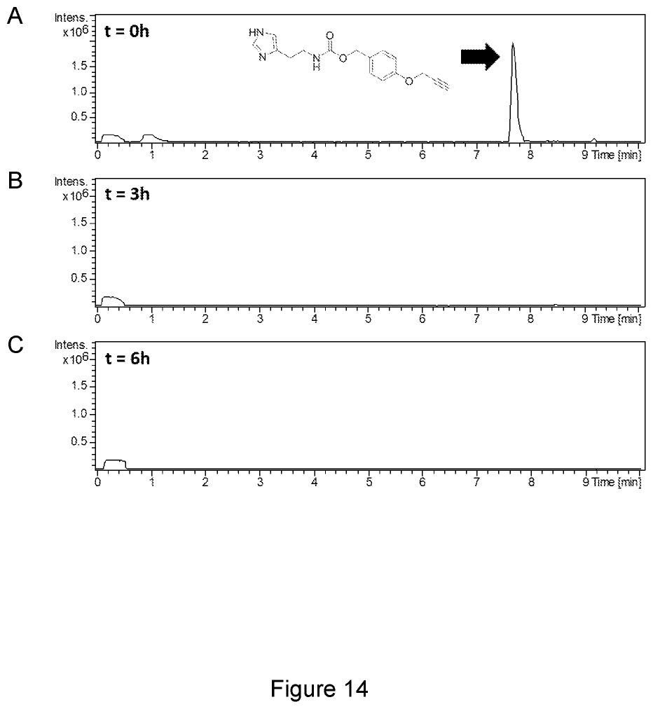

FIGS. 13 and 14 show the LCMS chromatograms for the Pd-catalysed deprotection of oPOBC-Hist and pPOBC-Hist. As seen in FIGS. 13 and 14, oPOBC-Hist and pPOBC-Hist completely disappeared from the crude mixture after 3 h of incubation with Pd⁰-beads.

Ninhydrin Test for Detection of Pd⁰-Mediated Deprotected Histamine

Histamine, oPOBC-Hist and pPOBC-Hist (100 μM) were incubated in phosphate buffered saline (PBS, 1 ml) with 1 mg of Pd⁰ resin for 24 h at 37° C. (Thermomixer, shaker speed: 1,200 rpm). After 24 h, PBS was removed and 300 μL of solution A (described below) and then, 100 μL of solution B (described below) were added to the Pd⁰-functionalized resins. Eppendorfs were then heated at 95° C. for 5 min. A negative test, indicating the absence of free primary amine (histamine), was communicated by a light yellow/orange solution. A positive test was indicated by a dark purple solution. Variations in the darkness of the solution reflect variations in amine concentration. Optical density (O.D.) was measured at the maximum of absorbance for ninhydrin purple-blue complex at 570 nm.

Reagent solution A. Phenol (40 g) is added to EtOH (10 mL) and the mixture was heated until complete dissolution of the phenol. A solution of KCN (65 mg) in water (100 mL) was added to pyridine (100 mL). Reagent solution B. A solution of ninhydrin (2.5 g) in absolute EtOH (50 mL) was prepared and maintained in a light-proof container, preferably under inert atmosphere.

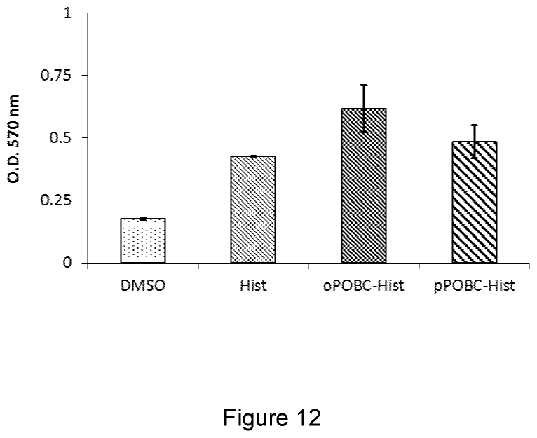

FIG. 12 shows the optical density of the solution mixture for control, Histamine, oPOBC-Hist and pPOBC-Hist by ninhydrin test. As expected, primary amines were detected in all samples due to the histamine release from the Pd-beads compared to the negative control (Pd-resin+DMSO).

Biological Activity

Human lung adenocarcinoma A549 cells, human glioblastoma U87G cells and T98 cells were chosen as models for the Vorinostat and Panobinostat antiproliferative studies; A549 cells, human prostate carcinoma DU145 cells and T98 cells were chosen as models for Doxorubicin; human pancreatic carcinoma MiaPaCa2 cells were chosen as a model for Gemcitabine; human pancreatic adenocarcinoma BxPC3 and human colorectal carcinoma HCT116 cells were chosen as models for 5FU; human glioblastoma U87G cells cells were chosen as models for the SN-38 and Etoposide antiproliferative studies; and human ovarian carcinoma A2780 cells were chosen as model for Olaparib. These cell lines were selected as these are primary malignancies against which the parental drugs are currently prescribed.

Prodrug Safety Studies

The toxicities of each active agent and prodrugs were compared by performing dose-response studies. Doses of Vorinostat and Vorinostat prodrugs (10, 30, 100, 200, 300, 400, 500 μM for A549, U87G and T98 cell lines); Panobinostat and POB-Panob (0.01, 0.03, 0.1, 0.3, 1, 3, 10, 30 and 100 μM for A549 cells); Doxorubicin and Doxorubicin prodrugs (0.003, 0.01, 0.03, 0.1, 0.3, 1, 3, 10, 30 μM for A549 and DU145 cell lines, and an additional dose of 100 μM for T98 cell line); Gemcitabine and Gemcitabine prodrugs (0.001, 0.003, 0.01, 0.03, 0.1, 0.3, 1, 3, 10 μM for MiaPaCa2 cells); 5FU and Bis-Pro-5FU (0.003, 0.01, 0.03, 0.1, 0.3, 1, 3, 10, 30 μM for BxPC3 and HCT116 cells); SN-38 and di-oPOB-SN-38 (0.03, 0.1 and 0.3 μM for U87G cells); Etoposide and Etoposide prodrugs (0.3, 1, 3, 10, 30 and 100 μM for U87G cells); and Olaparib and Prop-Olap (0.01, 0.03, 0.1, 0.3, 1, 3, 10, 30 and 100 μM for A2780 cells) were incubated with cells for 5 days and cell viability measured to determine the corresponding EC₅₀ values. Cell viability was analysed by fluoresce intensity (λₑₓ=540 nm; λₑₘ=590 nm) after 60-90 min incubation with PrestoBlue™ Reagent (Life Technologies).

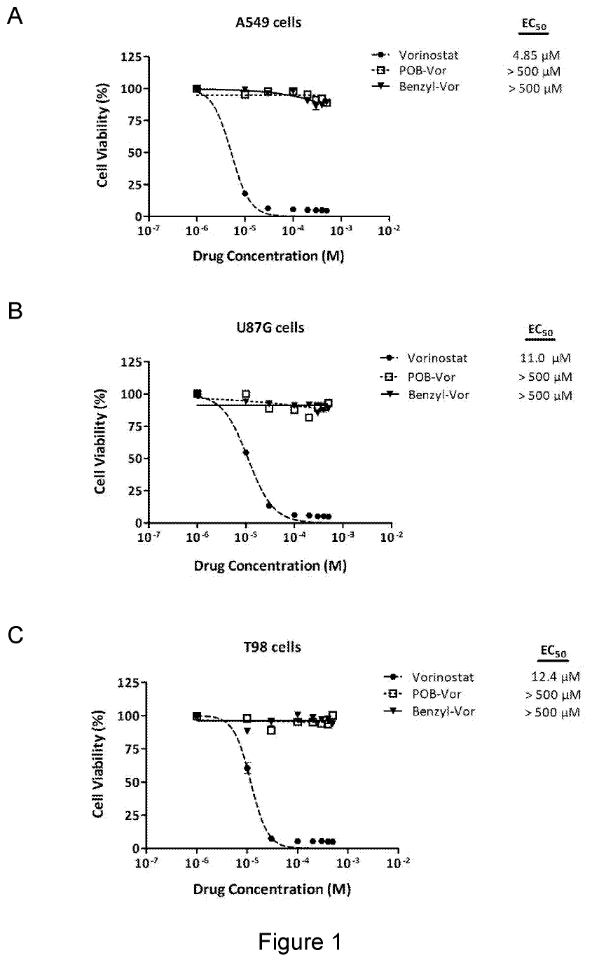

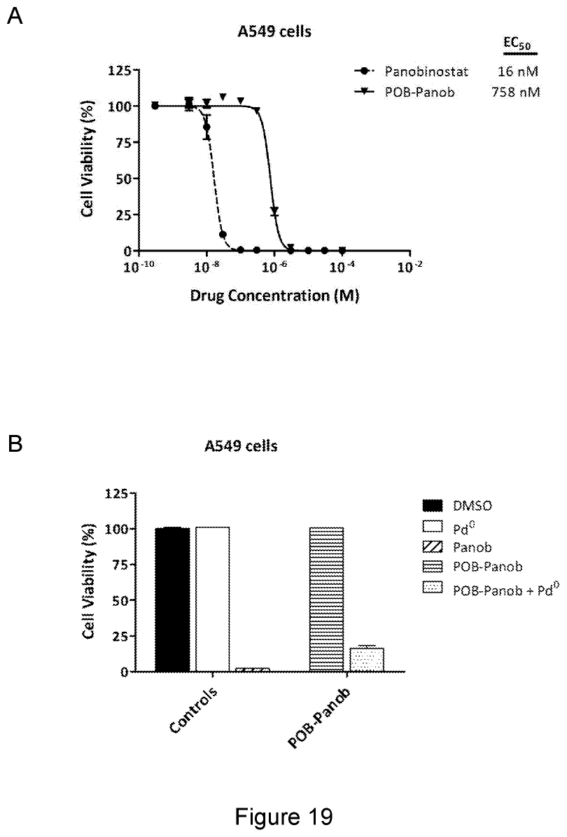

The cell viability data for A549, U87G and T98 cells are provided in FIG. 1. EC₅₀ values calculated for Vorinostat were 4.85 μM, 11 μM and 12.4 μM, respectively. Both POB-Vor and Benzyl-Vor display a significant reduction in the cytotoxic effect, showing an EC₅₀ value>500 μM for each cell line tested. The cell viability data for A549 cells are provided in FIG. 19 with EC₅₀ values calculated for Panobinostat and POB-Panob were 16 nM and 758 nM, respectively.

The cell viability data for A549, DU145 and T98 cells are provided in FIG. 4. EC₅₀ values calculated for Doxorubicin were 105 nM, 23 nM and 320 nM, respectively. Fold reduction ratios against Doxorubicin for pPOBC-Dox, oPOBC-Dox and Cbz-Dox were calculated as (1:66, 1:259, 1:89 [A549 cells]); (1:240, 1:310, 1:116 [DU145 cells]), (1:312, 1:312, 1:15 [T98]), respectively.

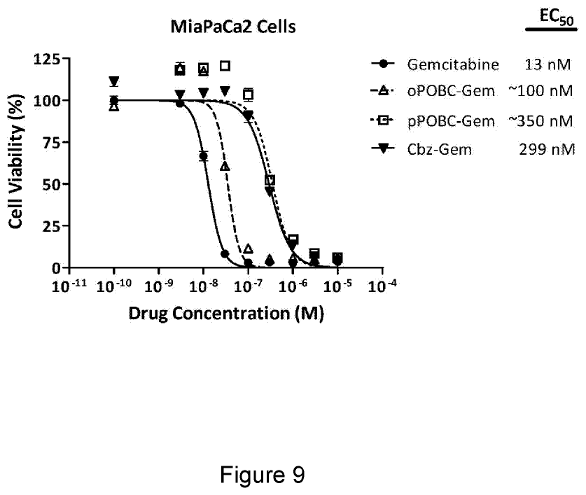

Cell viability data for MiaPaCa2 is provided in FIG. 9. EC₅₀ value calculated for Gemcitabine was 13 nM. pPOBC-Gem displays an intermediate reduction in the cytotoxic effect, showing a 27-fold reduction relative the parent active agent.

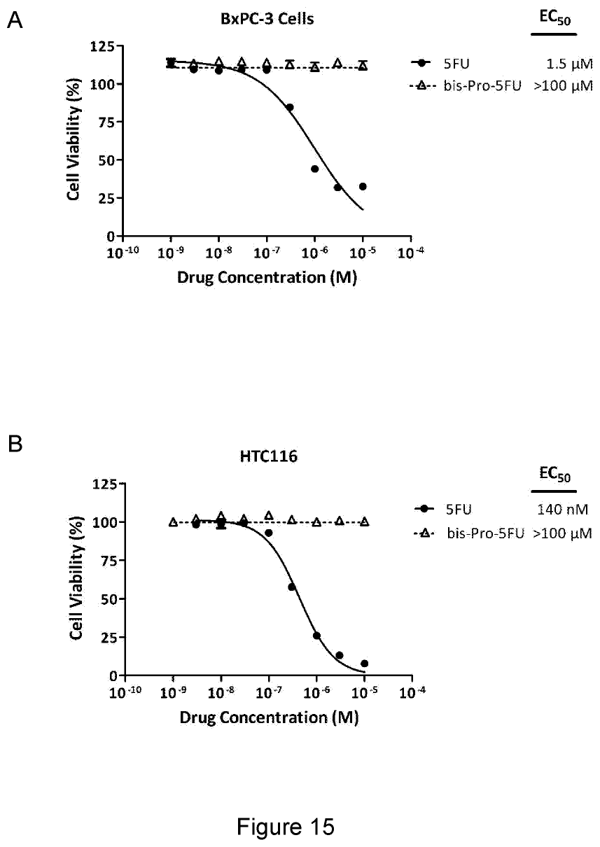

FIG. 15 shows the cell viability data for BxPC3 and HCT116 cells. EC₅₀ values calculated for 5FU were 140 nM and 1.5 μM, respectively. Bis-Pro-5FU shows a significant reduction in cytotoxic effect for both BxPC3 and HCT116 cells (EC₅₀ values=>100 μM).

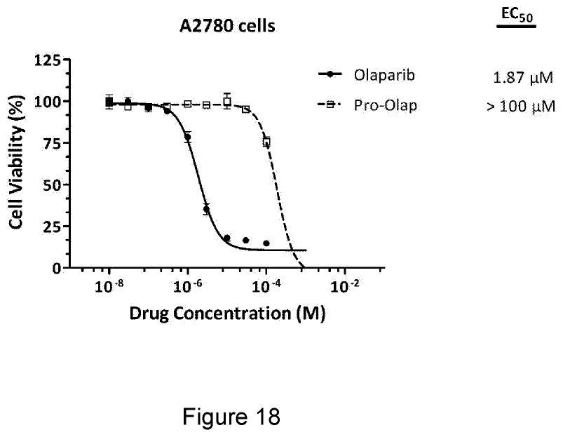

FIG. 18 shows the cell viability data for A2780 cells. EC₅₀ value calculated for Olaparib was 1.87 μM. Prop-Olap displays a significant reduction in the cytotoxic effect, showing an EC₅₀ value>100 μM for the cell line tested.

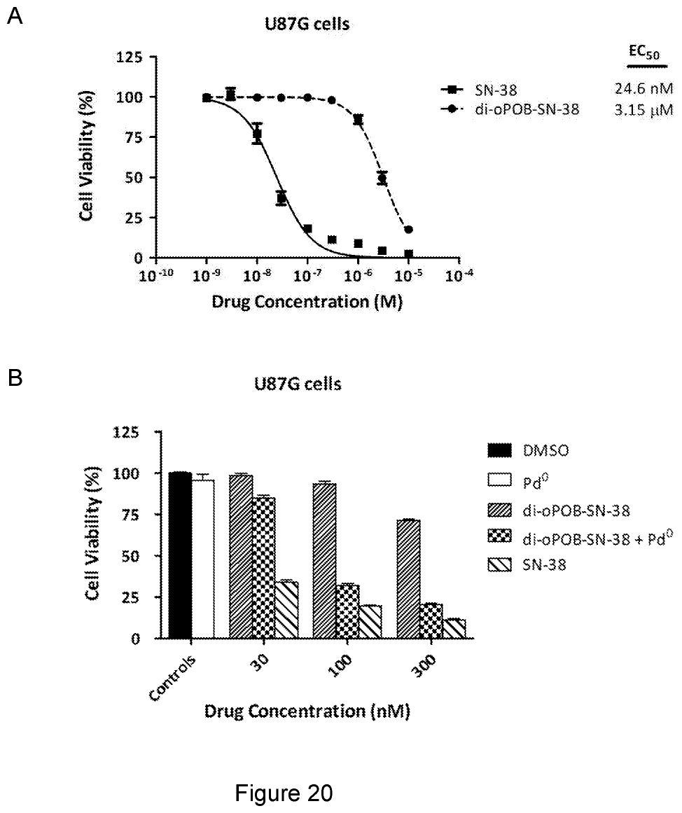

The cell viability data for U87G cells are provided in FIGS. 20-21. EC₅₀ values calculated for SN-38 and Etoposide were 24.6 nM and 4.97 μM, respectively. SN-38 prodrug shows an EC₅₀ value 3.15 μM for U87G cells. Etoposide prodrugs display a significant reduction in the cytotoxic effect, showing an EC₅₀ value>100 μM for each prodrug tested.

Generation of Drug from Prodrug in Cell Culture and Cytotoxic Effects

The toxigenic effect as a result of in situ generation of parental drug in cell culture was determined by incubating all cells in tissue culture media containing 0.1% (v/v) DMSO and a) Pd⁰, Au, or Pd/Au-resin (1 mg/mL for all cells tested, negative control); b) prodrug (negative control); or c) Pd⁰, Au, or Pd/Au-resin (1 mg/mL for all cells)+prodrug (reaction assay). Cells incubated in 0.1% (v/v) DMSO in media was used as an untreated cell reference standard (100% viability). A PrestoBlue® cell viability assay as described above was carried out and fluorescent intensities compared to the untreated cell control. Prodrug concentrations were 100 μM for Vorinostat prodrugs, 0.3 μM for Panobinostat prodrug, 1 μM for Doxorubicin prodrugs, 0.03 μM for Gemcitabine prodrugs for A549 cells and 3 μM (BxPC-3 cells) or 30 μM (HCT116 cells) for 5FU prodrugs or 100 μM (U87G cells) for SN-38 prodrug.

The combination of POB-Vor+Pd⁰ resins, oPOBC-Dox+Pd⁰ resins, pPOBC-Dox+Pd⁰ resins or pPOBC-Gem+Pd⁰ resins showed a strong toxigenic effect in A549 (POB-Vor); A549, DU145 and T98 (oPOBC-Dox and pPOBC-Dox) and MiaPaCa2 cell lines (pPOBC-Gem) A549 (POB-Panob) and U87G (di-oPOB-SN-38) as shown in FIGS. 4, 8, 11, 19 and 20 respectively, indicating the generation of Vorinostat, Doxorubicin or Gemcitabine to significant levels. Benzyl-Vor, Cbz-Dox or Cbz-Gem combined with Pd⁰-resins showed only low levels of toxicity in the different cell lines, which suggests the generation of low levels of parental drug.

Bis-Pro-5FU+Pd⁰ resins showed a strong toxigenic effect in both BxPC3 and HCT116 cells (FIG. 16), indicating the generation of 5FU to significant levels.

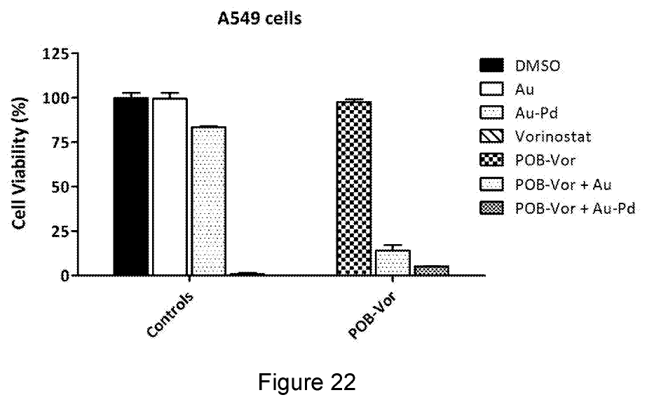

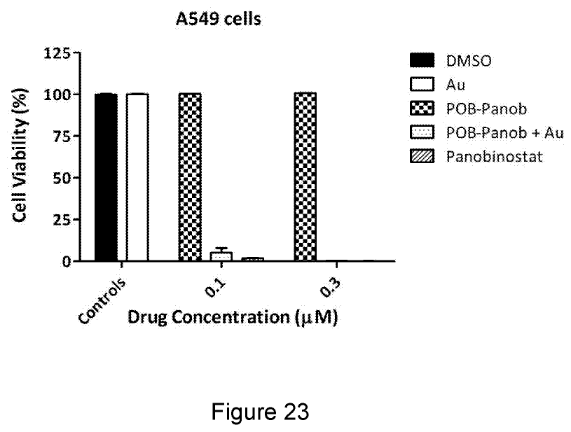

Combination of POB-Vor (100 μM)+Au or Pd/Au resins showed a strong toxigenic effect in A549 cells (FIG. 22), indicating the generation of Vorinostat to significant levels.

Dose Response Cell Viability Assay

To show extracellular efficacy of the palladium-mediated dealkylation of Vorinostat, Doxorubicin, Gemcitabine or 5FU prodrugs, a range of concentrations of POB-Vor, oPOBC-Dox, pPOBC-Dox, pPOBC-Gem, Bis-Pro-5FU and di-oPOB-SN-38 and Pd⁰-resins were incubated independently (negative controls) and in combination (BOOM conversion assay) at varying doses to study of proliferation cells in comparison to unmodified Vorinostat, Doxorubicin, Gemcitabine or 5FU, respectively (positive control). A dose response study was performed for each cell line keeping the quantity of Pd⁰ resin constant (0.8 mg/mL for U87G and 1 mg/mL for the rest of cell lines). All cells were plated in Dulbecco's Modified Eagle Media (DMEM) supplemented with serum (10% FBS) and L-glutamine (2 mM). Cells were seeded in a 96 well plate format (density: 1500 cells/mL for A549, 2000 cells/mL for U87G, 1000 cells/mL for T98, 2000 for DU145 cells, 1000 for MiaPaca2 cells, 2500 for BxPc3 cells and 3000 for HTC116 cells) and incubated for 48 h at 37° C. and 5% CO₂ before treatment. Each well was then replaced with fresh media containing: Pd⁰-resins (0.8 mg/mL for U87G and 1 mg/mL for the rest of cell lines, negative control); prodrug (1 μM to 100 μM for POB-Vor; 0.03 μM to 3 μM for Doxorubicin prodrugs in A459 and DU145 cells and 0.1 μM to 10 μM for T98 cells; 0.003 μM to 0.3 μM for Gemcitabine prodrugs; 0.03 μM to 3 μM for 5FU prodrug in BxPC3 cells and 0.3 μM to 30 μM in HTC116 cells and 0.03 μM to 0.3 μM for SN-38 prodrug in U87G) in DMSO (0.1% v/v) (negative control); active agent (concentrations as above) in DMSO (0.1% v/v), (positive control); or a combination of Pd⁰ resin+prodrug (concentrations as above in 0.1% v/v DMSO). Cells incubated in 0.1% (v/v) DMSO in media were used as untreated cell reference standard (i.e. 100% cell viability). Cells were incubated in the fresh media for 5 days. PrestoBlue cell viability reagent (Life Technologies) (10% v/v) was then added to each well and the plate incubated for 60-90 min. Fluorescence intensity values (detected using a PerkinElmer EnVision 2101 multilabel reader with excitation filter at 540 nm and emissions filter at 590 nm) were determined relative to the untreated cell control.

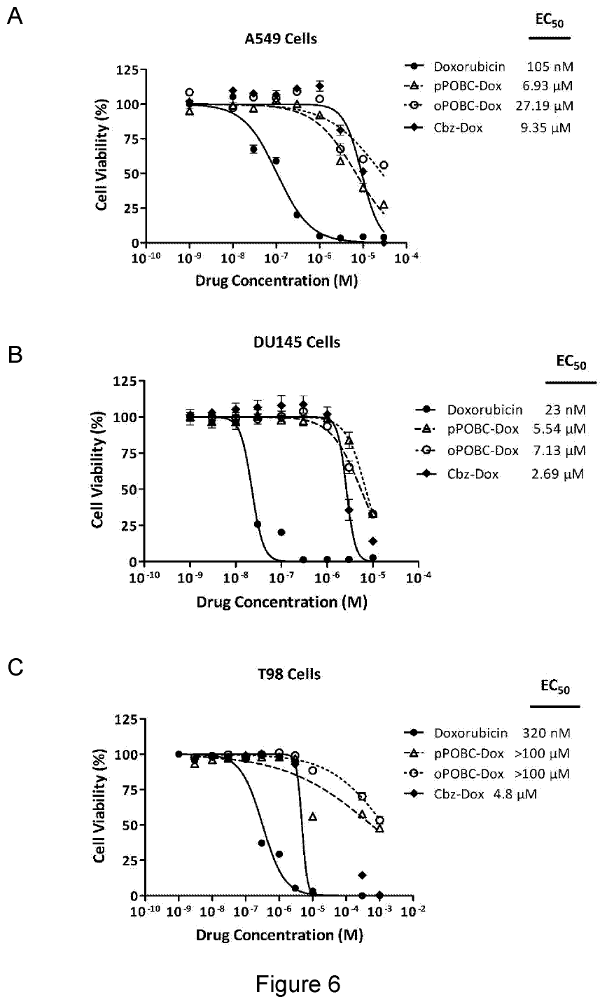

As shown in FIGS. 1, 4, 6, 8, 9 and 20, the prodrug/catalyst system for each Vorinostat, Doxorubicin, Gemcitabine, 5FU and SN-38 prodrugs showed significant cytotoxic effects at each concentration tested, and calculated EC₅₀ values similar to those calculated above for free active agent, as shown in Tables 1-3 (below):

TABLE 1

EC₅₀

| | | POB-Vor + |
|---|---|---|
| Cell line | Vorinostat | Pd-resins |
| A549 | 4.85 μM | 10.49 μM |
| U87G | 11 μM | 24.70 μM |
| T98 | 12.4 μM | 16.77 μM |

TABLE 2

EC₅₀

| | | oPOBC-Dox + | | pPOBC-Dox + | |
|---|---|---|---|---|---|
| Cell line | Doxorubicin | Pd-resins | | Pd-resins | |
| A549 | 105 nM | 81 | nM | 382 | nM |
| DU145 | 23 nM | 2.6 | μM | 1.1 | μM |
| T98 | 320 nM | 603 | nM | 360 | nM |

TABLE 3

EC₅₀

| | | pPOBC-Gem + |
|---|---|---|
| Cell line | Gemcitabine | Pd-resins |
| MiaPaCa2 | 13 nM | 226 nM |

### Conclusions

The data show that the compounds (i.e. prodrugs) of the invention can be deprotected in a controlled manner using biocompatible palladium and/or gold catalyst to generate free active agent in situ, which exhibits the desired biological activity. The data show that prodrugs of the invention are suitably non-toxic and do not interfere with the active agent pathway, thus providing ideal active agent precursors. Furthermore, the by-products produced in the deprotection reaction are also biocompatible (e.g. propargyl groups provide 1-hydroxyacetone as by-product, benzyl groups provide 1,2 or 1,4 hydroxybenzyl alcohol as by-products).

The precise spatial control of prodrug deprotection provided by palladium nad/or gold implants, along with lack of toxicity of the prodrug compounds means that prodrugs of the invention can be deprotected specifically at the disease site, which should thus reduce general systemic concentration of the free active agent. This is especially desirable in cancer treatments where side-effects resulting from the active agent acting non-specifically on other organs in the body can be severe. This may also in turn allow prodrugs of the invention to be administered in higher doses, providing higher concentrations of active agent at the disease site than would have been tolerated through general systemic administration of the active agent due to risk of the side-effects mentioned above.

It will be understood that the invention has been described by way of example only and modifications may be made whilst remaining within the scope and the spirit of the invention.

1. A method of preparing an active agent or a salt thereof, the method comprising the steps:
a) providing a first compound defined according to formula (1): 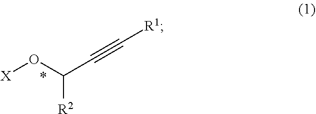
and
b) cleaving the bond (*) between the oxygen and the propargyl group under biologically compatible conditions by reacting the first compound with palladium or gold;
wherein R¹ and R² are independently selected from the group consisting of H, optionally substituted C₁-C₁₀ alkyl, optionally substituted C₃-C₁₀ cycloalkyl, optionally substituted C₂-C₁₀ alkenyl, optionally substituted C₃-C₁₀ cycloalkenyl, optionally substituted C₂-C₁₀ alkynyl, optionally substituted C₂-C₁₀ heteroalkyl, optionally substituted C₃-C₁₀ heterocycloalkyl, optionally substituted C₂-C₁₀ heteroalkenyl, optionally substituted C₃-C₁₀ heterocycloalkenyl, optionally substituted C₂-C₁₀ heteroalkynyl, optionally substituted C₆-C₁₄ aryl, optionally substituted C₅-C₁₄ heteroaryl,
wherein X—O comprises at least one aryl group or heteroaryl group directly connected to the oxygen (O) of the X—O substituent, and comprises the active agent or a salt thereof, and optionally comprises a linker between the oxygen and the active agent.

2. The method of claim 1, wherein the X—O group comprises a derivative of the active agent.

3. (canceled)

4. The method of claim 1, wherein the method is performed in a biological environment, such as in a cell, a tissue and/or a subject using a suitable palladium source and/or a suitable gold source.

5. The method of claim 1, wherein R¹ and R² are independently selected from the group consisting of H, optionally substituted C₁-C₅ alkyl, optionally substituted C₃-C₆ cycloalkyl, optionally substituted C₂-C₆ alkenyl, optionally substituted C₃-C₆ cycloalkenyl, optionally substituted C₂-C₅ alkynyl, optionally substituted C₂-C₅ heteroalkyl, optionally substituted C₃-C₆ heterocycloalkyl, optionally substituted C₂-C₅ heteroalkenyl, optionally substituted C₃-C₆ heterocycloalkenyl, optionally substituted C₂-C₅ heteroalkynyl, optionally substituted C₆-C₁₂ aryl, optionally substituted C₅-C₁₁ heteroaryl.

6. (canceled)

7. The method of claim 1, wherein the first compound has a general formula selected from the group comprising: 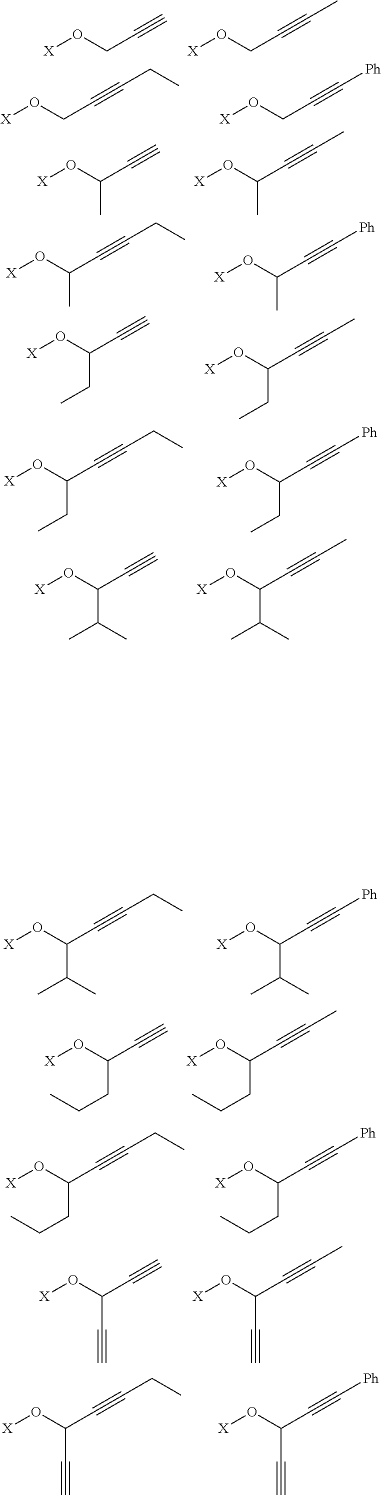
where the X—O group comprises an active agent and a linker, the active agent may be connected to the linker via an amine, hydroxyl, hydroxamic acid or carbonyl group of the active agent.

8. The method of claim 1, wherein the active agent contains a hydroxamic acid group connected to the propargyl group directly or via a linker.

9. The method of claim 8, wherein the active agent is vorinostat, belinostat, panobinostat, or derivatives thereof.

10. The method of claim 1, wherein the active agent contains one or more primary or secondary amino groups connected to the oxypropargyl group directly or via a linker.

11. The method of claim 10, wherein the active agent is doxorubicin, gemcitabine, histamine, mitoxantrone, panobinostat, hydroxyurea, paclitaxel, phosphoramide mustard, procarbazine, 5-(monomethyl triazine)-imidazole-4-carboxamide, dasatinib, erlotinib, bosutinib, gefitinib, lapatinib, vandetanib, pazopanib, crizotinib, ceritinib, afatinib, ibrutinib, dabrafenib, trametinib, palbociclib, spanisertib or derivatives thereof.

12. The method of claim 1, wherein the active agent comprises a phenolic OH connected to the oxypropargyl group directly or via a linker, including the equivalent lactam tautomers.

13. The method of claim 12, wherein the active agent is 5-fluorouracil (5-FU or 5FU), floxuridine, olaparib, permetrexed, sunitinib, nintedanib, doxorubicin, mitoxantrone, 4-hydroxytamoxifen, etoposide, duocarmycin or derivatives thereof.

14. The method of claim 1, wherein the compound according to formula (1) is selected from the following group:

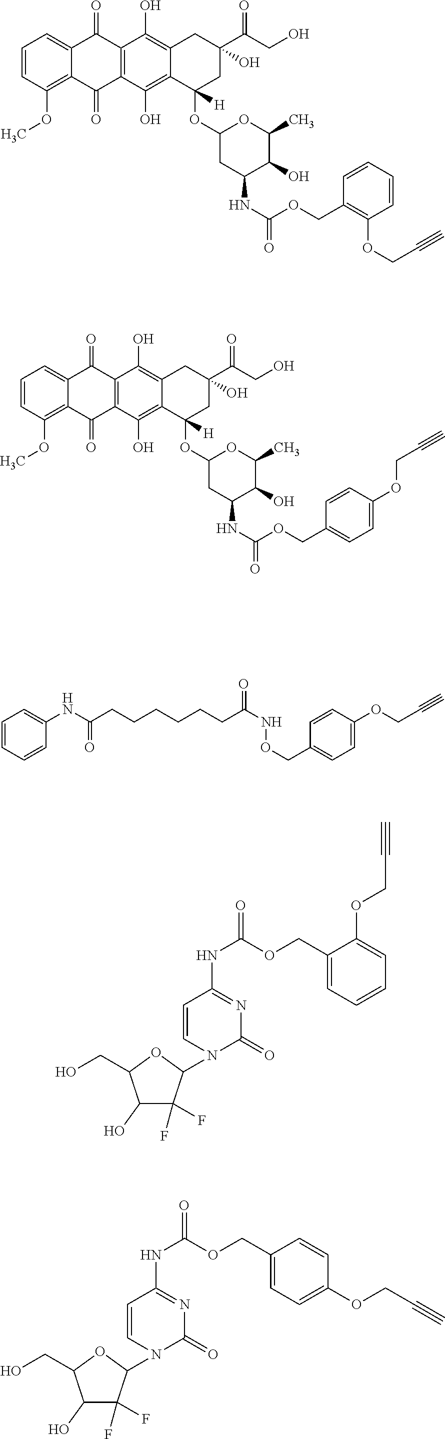 [SMILES: C#CCOc1ccccc1COC(=O)N[C@H]1CC(O[C@H]2C[C@](O)(C(=O)CO)Cc3c(O)c4c(c(O)c32)C(=O)c2c(OC)cccc2C4=O)O[C@@H](C)[C@H]1O ; C#CCOc1ccc(COC(=O)N[C@H]2CC(O[C@H]3C[C@](O)(C(=O)CO)Cc4c(O)c5c(c(O)c43)C(=O)c3c(OC)cccc3C5=O)O[C@@H](C)[C@H]2O)cc1 ; C#CCOc1ccc(CONC(=O)CCCCCCC(=O)Nc2ccccc2)cc1 ; C#CCOc1ccccc1COC(=O)Nc1ccn(C2OC(CO)C(O)C2(F)F)c(=O)n1 ; C#CCOc1ccc(COC(=O)Nc2ccn(C3OC(CO)C(O)C3(F)F)c(=O)n2)cc1]

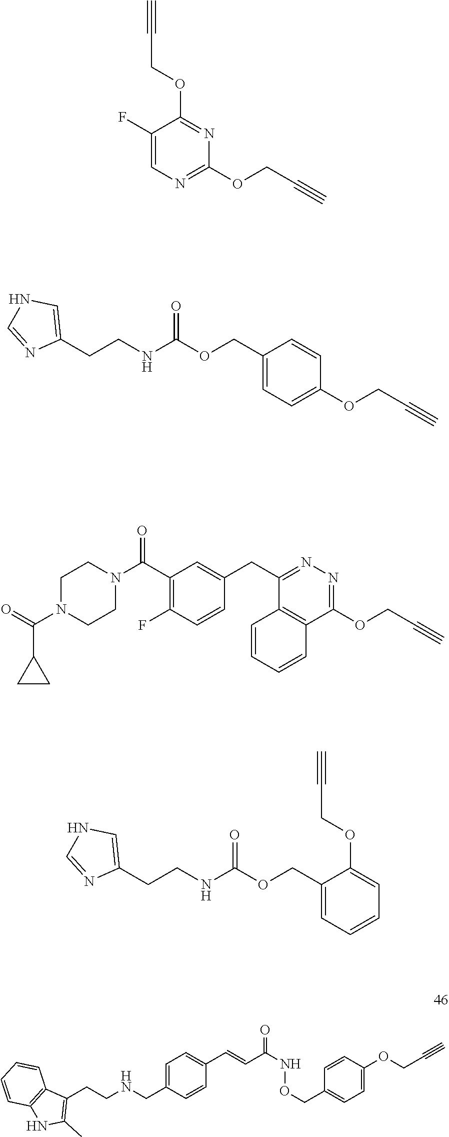 [SMILES: C#CCOc1ncc(F)c(OCC#C)n1 ; C#CCOc1ccc(COC(=O)NCCc2c[nH]cn2)cc1 ; C#CCOc1nnc(Cc2ccc(F)c(C(=O)N3CCN(C(=O)C4CC4)CC3)c2)c2ccccc12 ; C#CCOc1ccccc1COC(=O)NCCc1c[nH]cn1 ; C#CCOc1ccc(CONC(=O)/C=C/c2ccc(CNCCc3c(C)[nH]c4ccccc34)cc2)cc1]

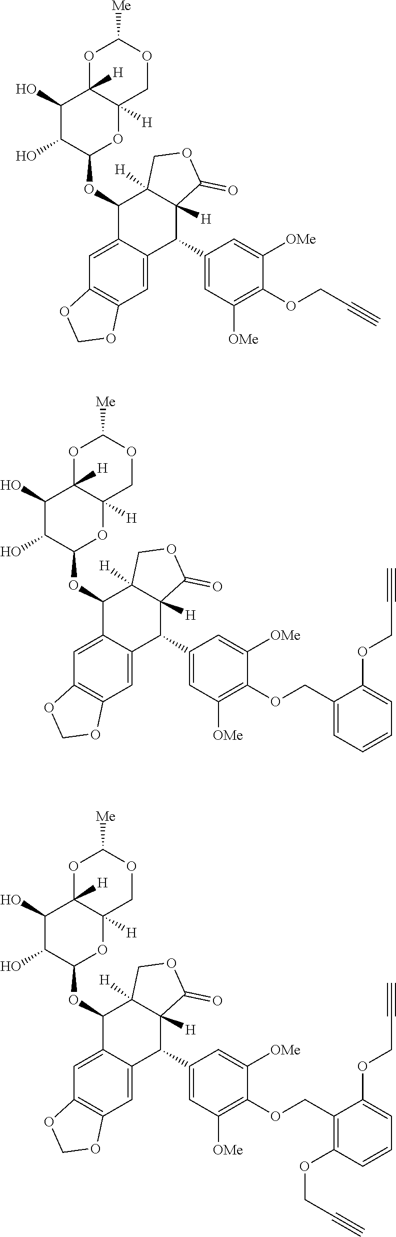

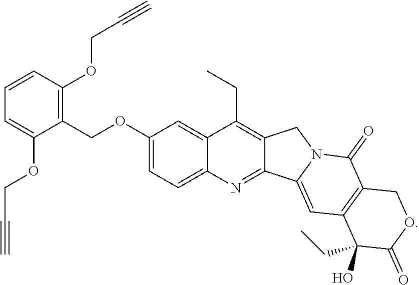 [SMILES: C#CCOc1cccc(OCC#C)c1COc1ccc2nc3c(c(CC)c2c1)Cn1c-3cc2c(c1=O)COC(=O)[C@]2(O)CC]

15. A first compound according to the general formula (1): 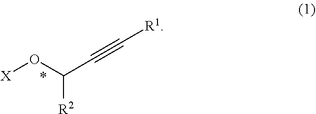
wherein R1 and R2 are independently selected from the group consisting of H, optionally substituted C1-C10 alkyl, optionally substituted C3-C10 cycloalkyl, optionally substituted C2-C10 alkenyl, optionally substituted C3-C10 cycloalkenyl, optionally substituted C2-C10 alkynyl, optionally substituted C2-C10 heteroalkyl, optionally substituted C3-C10 heterocycloalkyl, optionally substituted C2-C10 heteroalkenyl, optionally substituted C3-C10 heterocycloalkenyl, optionally substituted C2-C10 heteroalkynyl, optionally substituted C6-C14 aryl, optionally substituted C5-C14 heteroaryl,
wherein X—O comprises at least one aryl group or heteroaryl group directly connected to the oxygen (O) of the X—O substituent, and comprises an active agent or a salt thereof, and optionally comprises a linker between the oxygen and the active agent;
wherein the carbon-oxygen bond (*) is cleaved under biological conditions to release the active agent when the compound of formula (1) is reacted with palladium or gold.

16. The first compound of claim 15, wherein R1 and R2 are independently selected from the group consisting of H, optionally substituted C1-C5 alkyl, optionally substituted C3-C6 cycloalkyl, optionally substituted C2-C6 alkenyl, optionally substituted C3-C6 cycloalkenyl, optionally substituted C2-C5 alkynyl, optionally substituted C2-C5 heteroalkyl, optionally substituted C3-C6 heterocycloalkyl, optionally substituted C2-C5 heteroalkenyl, optionally substituted C3-C6 heterocycloalkenyl, optionally substituted C2-C5 heteroalkynyl, optionally substituted C6-C12 aryl, optionally substituted C5-C11 heteroaryl.

17. The first compound of claim 15, wherein the first compound has a general formula selected from the group comprising: 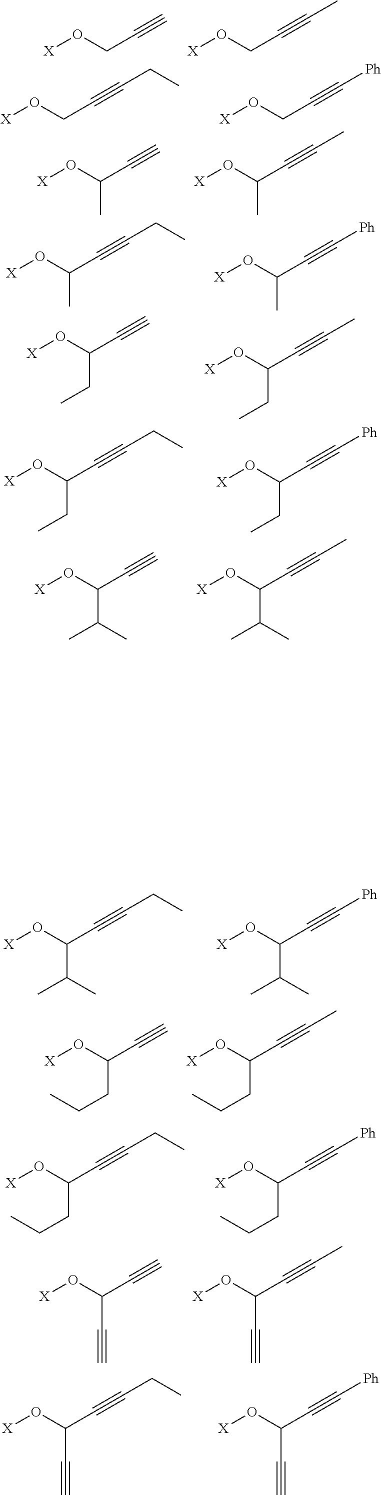
where the X—O group comprises an active agent and a linker, the active agent may be connected to the linker via an amine, hydroxyl or carbonyl group of the active agent.

18. The first compound of claim 15, wherein the linker is selected from the group 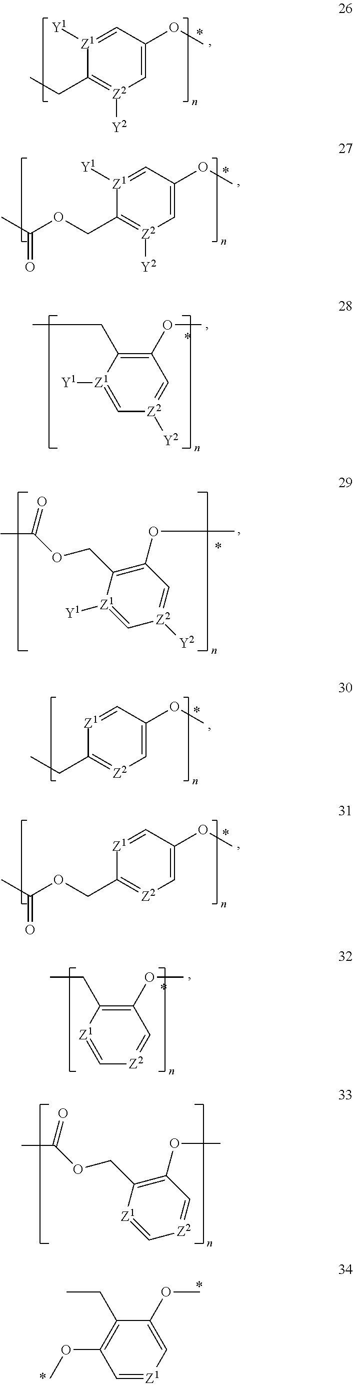 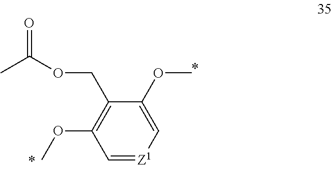
wherein
Z¹ and Z² are independently selected from N, CH, C;
Y¹ and Y² are independently selected from H, NO₂, halogen, COOR³, OR⁴;
R³ and R⁴ are independently selected from the group consisting of H, optionally substituted C₁-C₁₀ alkyl, optionally substituted C₃-C₁₀ cycloalkyl, optionally substituted C₂-C₁₀ alkenyl, optionally substituted C₃-C₁₀ cycloalkenyl, optionally substituted C₂-C₁₀ alkynyl, optionally substituted C₂-C₁₀ heteroalkyl, optionally substituted C₃-C₁₀ heterocycloalkyl, optionally substituted C₂-C₁₀ heteroalkenyl, optionally substituted C₃-C₁₀ heterocycloalkenyl, optionally substituted C₂-C₁₀ heteroalkynyl, optionally substituted C₆-C₁₄ aryl, optionally substituted C₅-C₁₄ heteroaryl; and
n is 1-10, preferably 1, 2 or 3.

19. The first compound according to claim 1 selected from the following group:

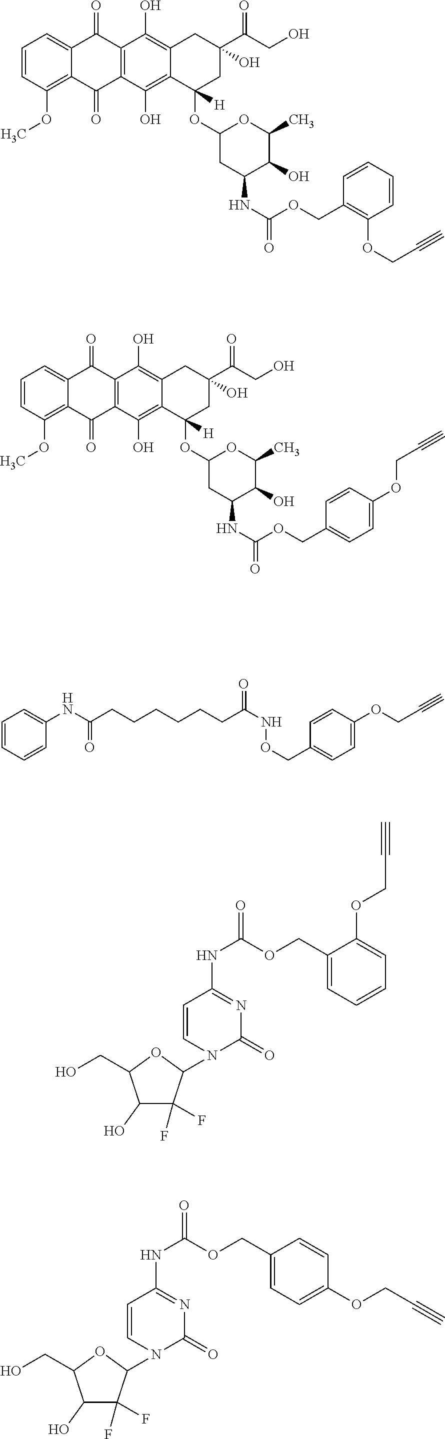 [SMILES: C#CCOc1ccccc1COC(=O)N[C@H]1CC(O[C@H]2C[C@](O)(C(=O)CO)Cc3c(O)c4c(c(O)c32)C(=O)c2c(OC)cccc2C4=O)O[C@@H](C)[C@H]1O ; C#CCOc1ccc(COC(=O)N[C@H]2CC(O[C@H]3C[C@](O)(C(=O)CO)Cc4c(O)c5c(c(O)c43)C(=O)c3c(OC)cccc3C5=O)O[C@@H](C)[C@H]2O)cc1 ; C#CCOc1ccc(CONC(=O)CCCCCCC(=O)Nc2ccccc2)cc1 ; C#CCOc1ccccc1COC(=O)Nc1ccn(C2OC(CO)C(O)C2(F)F)c(=O)n1 ; C#CCOc1ccc(COC(=O)Nc2ccn(C3OC(CO)C(O)C3(F)F)c(=O)n2)cc1]

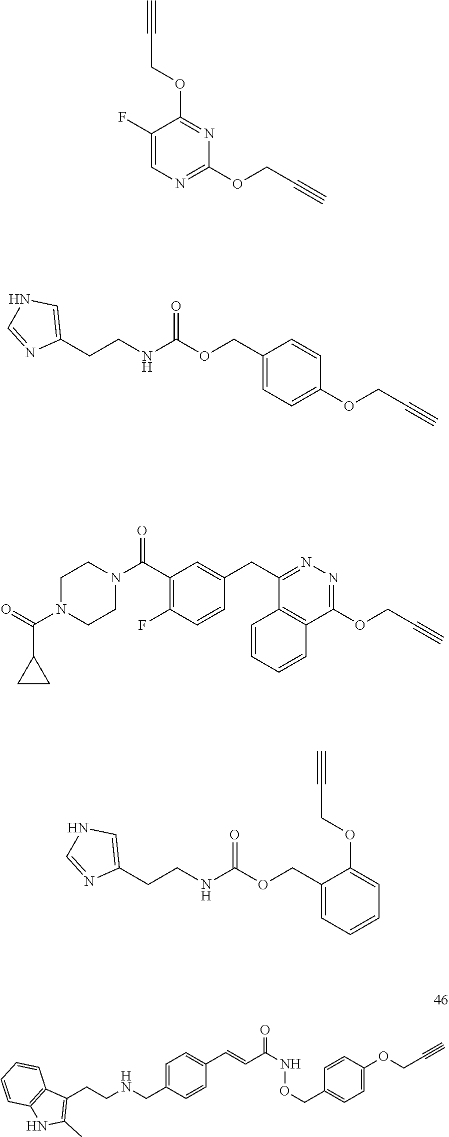 [SMILES: C#CCOc1ncc(F)c(OCC#C)n1 ; C#CCOc1ccc(COC(=O)NCCc2c[nH]cn2)cc1 ; C#CCOc1nnc(Cc2ccc(F)c(C(=O)N3CCN(C(=O)C4CC4)CC3)c2)c2ccccc12 ; C#CCOc1ccccc1COC(=O)NCCc1c[nH]cn1 ; C#CCOc1ccc(CONC(=O)/C=C/c2ccc(CNCCc3c(C)[nH]c4ccccc34)cc2)cc1]

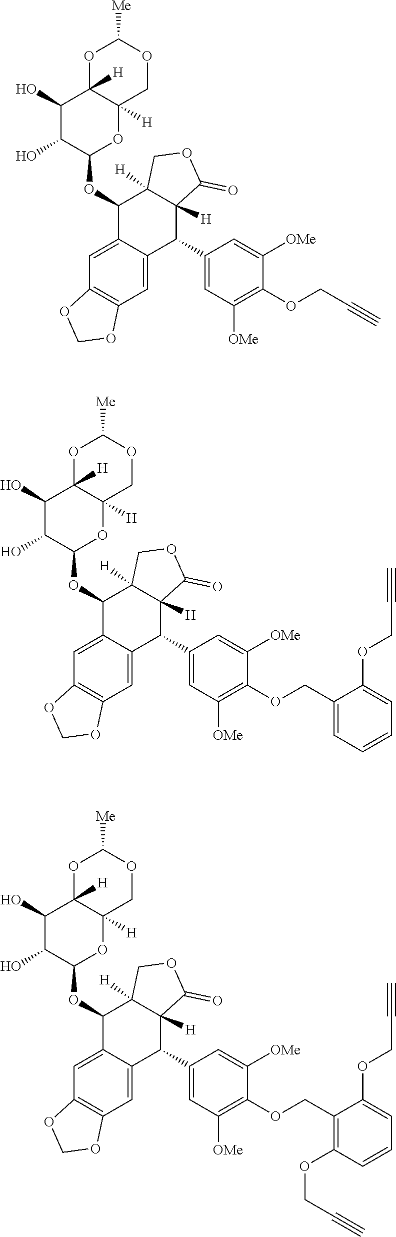

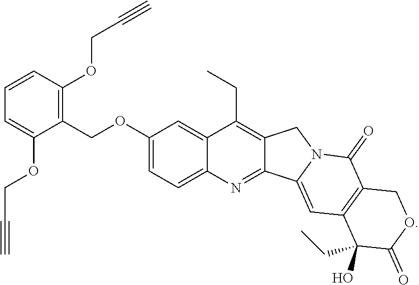 [SMILES: C#CCOc1cccc(OCC#C)c1COc1ccc2nc3c(c(CC)c2c1)Cn1c-3cc2c(c1=O)COC(=O)[C@]2(O)CC]

20. A method of treatment of disease by inserting an implant that comprises palladium and/or gold in a target area to be treated, and then delivering the first composition according to claim 15 to the target area, optionally wherein the disease is cancer.

21-23. (canceled)

24. An implant comprising palladium and/or gold for use in a method of treatment, wherein the method comprises administering a first compound or salt according to claim 1 or a pharmaceutically acceptable salt thereof and the implant to the subject.

25. The implant of claim 24 comprising palladium in particulate form and/or gold in particulate form embedded in a matrix.

26-31. (canceled)
